(19) United States
(12) Patent Application Publication
(10) Pub. No.: US 2025/0297326 A1
(43) Pub. Date: Sep. 25, 2025

(54) SNPS PANEL FOR KINSHIP IDENTIFICATION IN KOREAN AND USE THEREOF

(71) Applicant: REPUBLIC OF KOREA(NATIONAL FORENSIC SERVICE DIRECTOR MINISTRY OF INTERIOR AND SAFETY, Gangwon-do (KR)

(72) Inventors: Cho Hee KIM, Gangwon-do (KR); Kyungmyung LEE, Gangwon-do (KR); Suyeon YOO, Gangwon-do (KR); Byeong Ju YOUN, Gangwon-do (KR)

(21) Appl. No.: 18/850,720

(22) PCT Filed: Sep. 27, 2022
(86) PCT No.: PCT/KR2022/014443

§ 371 Date: Sep. 25, 2024

(30) Foreign Application Priority Data
May 12, 2022 (KR) 10-2022-0058633
May 12, 2022 (KR) 10-2022-0058635

(51) Int. Cl. C12Q 1/6888; C12Q 1/6809; C12Q 1/6827

(52) U.S. Cl. CPC C12Q 1/6888; C12Q 1/6809; C12Q 1/6827; C12Q 2600/156

(57) ABSTRACT

The present invention relates to information regarding an SNP panel for kinship identification in Korean and a use thereof. The composition for kinship identification in Korean of the present invention may be advantageously utilized to enable, even when no parent DNA is available, the use of only the minimum number of forensic SNP markers to clearly distinguish with respect to a subject, individuals in a first-degree relationship that is one of parent, child, brother, sister, and sibling, from individuals who are not in any first-degree relationship, or to provide information on individuals who are possibly in a first-degree relationship and individuals who may not be in any first-degree relationship.

### CROSS-REFERENCE TO RELATED APPLICATIONS

This application is a national phase application of PCT Application No. PCT/KR2022/014443, filed on 27 Sep. 2022, which claims the benefit and priority to Korean Patent Application Nos. 10-2022-0058633, filed on 12 May 2022, and 10-2022-0058635, filed on 12 May 2022. The entire disclosures of the applications identified in this paragraph are incorporated herein by references.

### SEQUENCE LISTING

This application contains references to amino acid sequences and/or nucleic acid sequences which have been submitted concurrently herewith as the sequence listing XML file entitled “000366usnp_SequenceListing.XML”, file size 2,007,040 bytes, created on 30 Apr. 2025. The aforementioned sequence listing is hereby incorporated by reference in its entirety pursuant to 37 C.F.R. § 1.52(e)(5).

### TECHNICAL FIELD

The present invention relates to an SNP (single nucleotide polymorphism) panel for kinship identification in Korean and a use thereof.

The present invention was made with the support of the Ministry of the Interior and Safety of the Republic of Korea under Project ID Number 1315001668 and Project Number NFS2021 DNA02, which was executed in the research project named “the Mid- to Long-Term Development Plan of Scientific Investigation Research and Development (R&D)” in the research project titled “Development and uses of SNP panels for kinship identification” by National Forensic Service, from 1 Jan. to 31 Dec. 2021.

### BACKGROUND ART

STR (short tandem repeats) is a concept originated from the gene of HLA (human leukocyte antigen, membrane protein of leukocytes), wherein 1 to 6 bases in the gene form a single motif and the motif is repeated. STRs are inherited through generations, are used to diagnose genetic diseases due to their relatively high polymorphic nature, have been continuously maintained and managed by CODIS (Combined DNA Index System) at Federal Bureau of Investigation (FBI), and are also genetic markers used to establish identity of a person and confirm familial relations.

Methods of establishing identity of a person or confirming familial relations by using such STRs involve measuring the size of an entire gene formed by various motifs via capillary electrophoresis, thereby estimating the number of motif repeats. However, these methods may lead to an inaccurate result if there are variants or mutations in an amplified individual gene, and may be unable to identify kinship when genes have a length of 300 bp or more in severely degraded samples with low DNA yield.

These limitations have been previously reported in the academia since early 2000 when the international human genome research was conducted, and various techniques to overcome such limitations have been suggested. Particularly, along with advancements made in the DNA sequencing technology, next generation sequencing (NGS) technology have been globally and gradually incorporated into and found applications in forensic investigations on a greater number of genetic variations.

Furthermore, the current analysis on corpses with unknown identity and missing children only allows one-on-one comparisons between an unknown corpse and the unknown corpse's guardian group, and between a missing child and the missing child's guardian, and paternity testing (‘1-chon’ which is parent-child relationship in the ‘chonsu’ system referring to the degree of kinship in Korea) through mutual search. For other relationships of higher degrees, only one-on-one comparison between specific individuals is possible, and one-to-many searches are not possible. In this context, there is a need to analyze the genome of Korean individuals and develop a minimum number of forensic SNP markers that enables identification of relationships of ‘2-chon’ or higher (the ‘2-chon’ is full sibling relationship or grandparent-grandchild relationship in the ‘chonsu’ system in Korea).

### DISCLOSURE

### Technical Problem

The present inventors have endeavoured to develop the minimum number of forensic SNP markers that enables kinship identification in a Korean population. As a result, from about 84 million SNPs of 88 unrelated Korean individuals disclosed in Korean National Standard Reference Variome (KoVariome) database, the present inventors have discovered 918 SNP markers and 482 SNP markers for kinship identification in a Korean population and demonstrated that by using these markers, in a group of Korean individuals who are in first- to fourth-degree relationships, it was possible to clearly distinguish with respect to a test person, individuals who are in a first-degree relationship as one of parent, child, brother, sister, and sibling, from individuals who are not in any first-degree relationship, and even in the absence of parent DNA information, it was possible to distinguish, with respect to a test person, individuals who are in any one of relationships as brother, sister, and sibling, from those who are not in any of such relationships. By demonstrating the above, the present inventors have arrived at the SNP panels for kinship identification in Korean.

Accordingly, a purpose of the present invention is to provide an SNP panel for kinship identification in Korean.

Another purpose of the present invention is to provide a composition or kit for kinship identification in Korean comprising the above-described SNP panel.

Still another purpose of the present invention is to provide a method of kinship identification in Korean, comprising identifying the nucleotide at the above-described SNP.

### Technical Solution

According to one aspect of the present invention, the present invention provides a composition for kinship identification in Korean, the composition comprising:
1) an agent for amplifying or detecting a single nucleotide polymorphism (SNP) located at position 101 in at least one sequence selected from the group consisting of nucleotide sequences set forth as SEQ ID NO: 1 to SEQ ID NO: 918;
2) an agent for amplifying or detecting a single nucleotide polymorphism (SNP) located at position 101 in at least one sequence selected from the group consisting of nucleotide sequences set forth as SEQ ID NO: 919 to SEQ ID NO: 1400; or
3) an agent for amplifying or detecting a single nucleotide polymorphism (SNP) located at position 101 in at least one sequence selected from the group consisting of nucleotide sequences set forth as SEQ ID NO: 1 to SEQ ID NO: 1400.

The present inventors have endeavoured to develop the minimum number of forensic SNP markers that enables kinship identification in a Korean population. As a result, from about 84 million SNPs of 88 unrelated Korean individuals disclosed in Korean National Standard Reference Variome (KoVariome) database, the present inventors have discovered 918 SNP markers and 482 SNP markers for kinship identification in a Korean population and demonstrated that by using these markers, in a group of Korean individuals who are in first- to fourth-degree relationships, it was possible to clearly distinguish with respect to a test person, individuals who are in a first-degree relationship as one of parent, child, brother, sister, and sibling, from individuals who are not in any first-degree relationship, and even in the absence of parent DNA information, it was possible to distinguish, with respect to a test person, individuals who are in any one of relationships as brother, sister, and sibling, from those who are not in any of such relationships.

Therefore, the composition for kinship identification in Korean according to the present invention may be utilized to resolve cases that the prior art STR technology used for the purpose of identification was unable to resolve, such as when brother-brother, sister-sister, or brother-sister relationships need to be identified without information of parents, when there are mutations within individual CODIS-23 loci, when DNA in the sample to be analyzed is severely fragmented due to skeletonization or putrefaction, and when the genetic distance between the test sample and the surviving family members is large. In addition, unlike the conventional STR techniques, since NGS technology enables simultaneous identification of multiple SNPs on the chromosome in multiple samples, the composition for kinship identification in Korean of the present invention is expected to play a significant role in establishing identity of multiple victims in massive disaster events when such a need arises.

In particular, while when using STR markers, complete replication of a motif having a size of 80 bp to 400 bp at a gene locus is necessary to identify the accurate allele type of the corresponding gene locus, when using the composition for kinship identification in Korean according to the present invention, since a single base for each SNP marker needs to be identified, kinship identification can be made even when the sample to be analyzed is in a skeletonized state or severely putrefied, thus rendering the DNA in the sample severely fragmented.

Also, since there have been many reports for individuals having a mutation within CODIS-23 loci being reported to have a different allele type from the parents' generation even when it is clear that they are biologically related, estimation of kinship using STR technology may be limited at CODIS-23 loci. However, the 918-SNP panel and the 482-SNP panel according to the present invention are selected by excluding SNP gene loci in repeated regions, and thus can be used to estimate kinship even when there is mutations within CODIS-23 loci.

In particular, compared to kinship testing using the conventional STR markers or about 10⁵ to 10⁶ SNPs, the composition for kinship identification in Korean according to the present invention is practical in forensic applications in that it permits the use of only 918 and/or 482 SNP markers to distinguish, with respect to a test person, first-degree relatives from those who are not first-degree relatives in a Korean population.

The terms “nucleotide sequence analysis”, “sequencing”, and “genome decoding” as used herein have no intended distinction and are used interchangeably in this specification.

The term “single nucleotide polymorphism (SNP)” refers to a variation of a single base at a specific position in the genome. The SNP is intended to encompass variations of a specific single base to another base at the same position in the genome of several individuals.

The term “panel” as used herein refers to a set of specific markers.

The term “SNP panel” as used herein refers to a set of specific SNP markers.

The term “whole genome sequencing (WGS)” as used herein refers to a method of determining the exact sequence of nucleotides of a genome, which is the sum total of genetic material of a cell or an organism.

In an embodiment of the present invention, the composition comprises an agent for amplifying or detecting an SNP located at position 101 in a sequence selected from the group consisting of nucleotide sequences set forth as SEQ ID NO: 1 to SEQ ID NO: 918.

In another embodiment of the present invention, the composition comprises an agent for amplifying or detecting an SNP located at position 101 in at least 100, at least 200, at least 300, at least 400, at least 500, at least 600, at least 700, at least 800, at least 900, or 918 sequences in a sequences selected from the group consisting of nucleotide sequences set forth as SEQ ID NO: 1 to SEQ ID NO: 918, but these numbers are only exemplary and are not limited thereto.

In an embodiment of the present invention, the composition further comprises an agent for amplifying or detecting an SNP located at position 101 in a sequence selected from the group consisting of nucleotide sequences set forth as SEQ ID NO: 919 to SEQ ID NO: 1400.

In another embodiment of the present invention, the composition comprises an agent for amplifying or detecting an SNP located at position 101 in at least 50, at least 100, at least 150, at least 200, at least 250, at least 300, at least 350, at least 400, at least 450, or 482 sequences in nucleotide sequences selected from the group consisting of nucleotide sequences set forth as SEQ ID NO: 919 to SEQ ID NO: 1400, but these numbers are only exemplary and are not limiting to.

In another embodiment of the present invention, the composition comprises an agent for amplifying or detecting an SNP located at position 101 in a sequence selected from the group consisting of nucleotide sequences set forth as SEQ ID NO: 1 to SEQ ID NO: 1400.

In an embodiment of the present invention, the composition comprises an agent for amplifying or detecting an SNP located at position 101 in at least 100, at least 200, at least 300, at least 400, at least 500, at least 600, at least 700, at least 800, at least 900, or 918 sequences selected from the group consisting of nucleotide sequences set forth as SEQ ID NO: 1 to SEQ ID NO: 918; and/or an agent for amplifying or detecting an SNP located at position 101 in at least 50, at least 100, at least 150, at least 200, at least 250, at least 300, at least 350, at least 400, at least 450, or 482 sequences selected from the group consisting of nucleotide sequences set forth as SEQ ID NO: 919 to SEQ ID NO: 1400, but these numbers are only exemplary and are not limiting to.

In another embodiment of the present invention, the composition comprises an agent for amplifying or detecting an SNP located at position 101 in at least 100, at least 200, at least 300, at least 400, at least 500, at least 600, at least 700, at least 800, at least 900, at least 1000, at least 1100, at least 1200, at least 1300, or 1400 sequences selected from the group consisting of nucleotide sequences set forth as SEQ ID NO: 1 to SEQ ID NO: 1400.

The SNP can be extracted from a human reference genome, such as GRCh37/hg19 or GRCh38/hg38.

The term “reference genome” as used herein refers to a standard sequence that is completely sequenced and established as a public database.

In an embodiment of the present invention, the SNP is not located within a region of genome functional element, or between 100 kbp (kilo base pair) upstream and 100 kbp downstream therefrom.

In a specific embodiment of the present invention, the genome functional element is an exon or a coding sequence. That is, the SNP of the present invention is not located in exons or coding sequences.

In another specific embodiment of the present invention, the SNP is not located within an exon or a coding sequence, or between 100 kbp upstream and 100 kbp downstream therefrom.

In an embodiment of the present invention, the SNP has a p value of 0.05 or more from Hardy-Weinberg equilibrium (HWE) testing. In another embodiment of the present invention, the SNP has a p value of more of 0.05 from HWE testing.

In an embodiment of the present invention, the SNP is extracted from KoVariome, which is Korea National Standard Reference Variome database, but is not necessarily limited thereto. Details on KoVariome are disclosed in Kim J. et al. (KoVariome: Korean National Standard Reference Variome database of whole genomes with comprehensive SNV, indel, CNV, and SV analyses. Sci Rep. 2018 Apr. 4; 8(1):5677).

In an embodiment of the present invention, the variant allele frequency in Korean population with respect to the SNP is 0.3 to 0.7. In another embodiment of the present invention, the variant allele frequency in Korean population with respect to the SNP is 0.4 to 0.6.

In a specific embodiment of the present invention, the SNP is an SNP having a variant allele frequency in Korean population of 0.3 to 0.7, or 0.4 to 0.6, extracted from KoVariome which is Korea National Standard Reference Variome database.

The term “variant allele frequency (VAF)” as used herein refers to a frequency at which the alleles are observed at particular loci in the genome. For the purpose of the present invention, the term variant allele frequency refers to a frequency at which a variant allele appears at a particular gene locus specific to the genome in a Korean population.

In an embodiment of the present invention, the SNP is not located in linkage disequilibrium (LD). In another embodiment of the present invention, the SNP excludes SNPs located in LD, which are excluded by using HaploReg v 4.1 database (Ward, L. D., & Kellis, M. (2016). HaploReg v 4: systematic mining of putative causal variants, cell types, regulators and target genes for human complex traits and disease. Nucleic Acids Res., 44(D1), D877-D881. http://compbio.mit.edu/HaploReg). In one specific embodiment of the present invention, the excluding SNPs located in LD by using HaploReg v 4.1 database is excluding SNPs having an r² value of 0.2 or more.

In an embodiment of the present invention, the SNP is not located in repeated regions in the genome known in the art. In another embodiment of the present invention, the SNP excludes SNPs located in repeated regions disclosed in www.repeatmasker.org/species/hg.html.

In an embodiment of the present invention, the agent is a primer, a probe, or a mixture thereof.

The term “primer” as used herein refers to an oligonucleotide, which is capable of acting as a point of initiation of synthesis when placed under conditions in which synthesis of primer extension product complementary to a nucleic acid strand (template) is induced, i.e., in the presence of nucleotides and an agent for polymerization, such as DNA polymerase, and at a suitable temperature and pH.

The term “probe” as used herein refers to a single-stranded nucleic acid molecule including a portion or portions that are substantially complementary to a target nucleic acid sequence. The probe may be labeled with a fluorescent material and/or a quencher.

The reporter molecule and the quencher molecule useful in the present invention may include any molecules known in the art, for example, following molecules (the numeric in parenthesis is a maximum emission wavelength in nanometer): Cy2™(506), YOPRO™-1(509), YOYO™-1(509), Calcein(517), FITC(518), FluorX™(519), Alexa™(520), rhodamine 110(520), 5-FAM(522), Oregon Green™500(522), Oregon Green™488(524), RiboGreen™(525), RhodamineGreen™(527), Rhodamine 123(529), Magnesium Green™(531), Calcium Green™(533), TO-PRO™-1(533), TOTO1(533), JOE(548), BODIPY530/550(550), Dil(565), BODIPY TMR(568), BODIPY558/568(568), BODIPY564/570(570), Cy3™(570), Alexa™546(570), TRITC(572), Magnesium Orange™(575), Phycoerythrin R&B(575), Rhodamine Phalloidin(575), Calcium Orange™(576), Pyronin Y(580), RhodamineB(580), TAMRA(582), Rhodamine Red™(590), Cy3.5™(596), ROX(608), Calcium Crimson™(615), Alexa™594(615), Texas Red(615), Nile Red(628), YO-PRO™_3(631), YYO™-3(631), Rphycocyanin(642), CPhycocyanin(648), TO-PRO™-3(660), TOTO3(660), DiD DiIC(5)(665), Cy5™(670) Thiadicarbocyanine(671), Cy5.5(694), HEX(556), TET(536), VIC(546), BHQ-1(534), BHQ-2(579), BHQ-3(672), BiosearchBlue(447), CAL Fluor Gold 540(544), CAL Fluor Orange 560(559), CAL Fluor Red 590(591), CAL FluorRed 610(610), CAL Fluor Red 635(637), FAM(520), Fluorescein(520), Fluorescein-C3(520), Pulsar 650(566), Quasar 570(667), Quasar 670(705), Quasar 705(610), and TxR(592).

Suitable pairs of reporter-quencher are disclosed in a variety of publications as follows: Pesce et al., editors, FLUORESCENCE SPECTROSCOPY (Marcel Dekker, New York, 1971); White et al., FLUORESCENCE ANALYSIS: A PRACTICAL APPROACH (Marcel Dekker, New York, 1970); Berlman, HANDBOOK OF FLUORESCENCE SPECTRA OF AROMATIC MOLECULES, 2nd EDITION (Academic Press, New York, 1971); Griffiths, COLOUR AND CONSTITUTION OF ORGANIC MOLECULES (Academic Press, New York, 1976); Bishop, editor, INDICATORS (Pergamon Press, Oxford, 1972); Haugland, HANDBOOK OF FLUORESCENT PROBES AND RESEARCH CHEMICALS (Molecular Probes, Eugene, 1992); Pringsheim, FLUORESCENCE AND PHOSPHORESCENCE (Interscience Publishers, New York, 1949); Haugland, R. P., HANDBOOK OF FLUORESCENT PROBES AND RESEARCH CHEMICALS, Sixth Edition, Molecular Probes, Eugene, Oreg., 1996; U.S. Pat. Nos. 3,996,345 and 4,351,760.

The “target nucleic acid”, “target nucleic acid sequence”, or “target sequence” refers to a nucleic acid sequence sought to be detected, and is annealed or hybridized with a primer or a probe under hybridization, annealing or amplification conditions.

More specifically, the probe and primer are single-stranded deoxyribonucleotide molecules. The probes or primers used in this invention may include naturally occurring dNMP (i.e., dAMP, dGM, dCMP and dTMP), modified nucleotide, or non-naturally occurring nucleotide. The probes or primers may also include ribonucleotides.

The primer must be sufficiently long to prime the synthesis of extension products in the presence of the agent for polymerization. The exact length of the primers depends on multiple factors, including temperature, the field of application, and the source of primer.

The term “annealing” or “priming” as used herein refers to the apposition of an oligodeoxynucleotide or nucleic acid to a template nucleic acid, whereby the apposition enables the polymerase to polymerize nucleotides into a nucleic acid molecule which is complementary to the template nucleic acid or a portion thereof.

A primer used in the present invention is hybridized or annealed to a portion of the template to form a double-stranded structure. Conditions for nucleic acid hybridization suitable for forming such a double-stranded structure are disclosed in Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

The term used “hybridizing” used herein refers to the formation of a double-stranded nucleic acid from complementary single stranded nucleic acids. The hybridization may occur between two nucleic acid strands perfectly matched or substantially matched with some mismatches. The complementarity for hybridization may depend on hybridization conditions, particularly temperature. As used herein, there is no intended distinction between the terms “annealing” and “hybridizing”, and these terms will be used interchangeably.

In an embodiment of the present invention, the SNP is amplified or detected by the primer or probe.

By using the primer and/or probe, a nucleotide sequence containing the SNP according to the present invention may be amplified or detected.

The application is performed by amplification of a gene.

In an embodiment of the present invention, the amplification of a gene is performed by a polymerase chain reaction (PCR) method.

In an embodiment of the present invention, the PCR simultaneously amplifies or detects an SNP located at 101 base position in at least 1, at least 10, at least 100, at least 200, at least 300, at least 400, at least 500, at least 600, at least 700, at least 800, at least 900, or 918 sequences selected from the group consisting of nucleotide sequences set forth as SEQ ID NO: 1 to SEQ ID NO: 918.

In an embodiment of the present invention, the PCR simultaneously amplifies or detects an SNP located at 101 base position in at least 1, at least 10, at least 50, at least 100, at least 150, at least 200, at least 250, at least 300, at least 350, at least 400, at least 450, or 482 sequences selected from the group consisting of nucleotide sequences set forth as SEQ ID NO: 919 to SEQ ID NO: 1400.

In another embodiment of the present invention, the PCR simultaneously amplifies or detects an SNP located at 101 base position in at least 1, at least 10, at least 100, at least 200, at least 300, at least 400, at least 500, at least 600, at least 700, at least 800, at least 900, at least 1,000, at least 1,100, at least 1,200, at least 1,300, or 1,400 sequences selected from the group consisting of nucleotide sequences set forth as SEQ ID NO: 1 to SEQ ID NO: 1400.

The PCR is the most well-known method of amplification of nucleic acids and has many variations and applications developed. For example, to enhance PCR specificity or sensitivity, variations of the conventional PCR protocol have been developed, examples of such variations being touchdown PCR, hot start PCR, nested PCR, and booster PCR. Furthermore, real-time PCR, differential display PCR (DD-PCR), rapid amplification of cDNA ends (RACE), multiplex PCR, inverse polymerase chain reaction (IPCR), vectorette PCR, thermal asymmetric interlaced PCR (TAIL-PCR), and multiplex PCR have been developed for specific applications. Details of the PCR are described in McPherson, M. J. and Moller, S. G. PCR. BIOS Scientific Publishers, Springer-Verlag New York Berlin Heidelberg, N.Y. (2000), and the teachings thereof are incorporated herein by reference.

The term “multiplex PCR” as used herein refers to a simultaneous amplification of multiple targets by a polymerase chain reaction in a reaction vessel.

In the polymerase chain reaction, various DNA polymerases may be used, and such DNA polymerases include Klenow fragment of E. coli DNA polymerase I, a thermostable DNA polymerase and bacteriophage T7 DNA polymerase. In particular, the polymerase is a thermostable DNA polymerase which may be obtained from a variety of bacterial species, including Thermus aquaticus (Taq), Thermus thermophilus (Tth), Thermus filiformis, Thermis flavus, Thermococcus literalis, and Pyrococcus furiosus (Pfu).

In an embodiment of the present invention, the composition comprises a Taq DNA polymerase or a Pfu DNA polymerase.

Products from the PCR may be replicated by rolling circle replication (RCA).

By using the products from the PCR, DNA nanoball sequencing may be performed.

As used herein, the term “DNA nanoball sequencing” refers to a high throughput sequencing technology that is used to analyze the entire genomic sequence by using RCA (rolling circle replication) to amplify DNA fragments to form a DNA concatemer in which DNA copies concatenate head to tail in a long strand and are compacted.

In an embodiment of the present invention, the agent for amplifying or detecting the SNP may be a primer, an oligomer, an adapter, a barcode sequence, or an index sequence for sequencing.

In an embodiment of the present invention, the primer, oligomer, adaptor, barcode sequence, or index sequence for sequencing may include at least 10 consecutive nucleotides that comprise an SNP located at 101 base position in nucleotide sequences set forth in SEQ ID NO: 1 to SEQ ID NO: 918 and/or SEQ ID NO: 919 to SEQ ID NO: 1400, or may comprise a nucleotide sequence complementary thereto.

The adaptor for sequencing may be designed so as to be complementary to a sequence coating a flow cell for sequencing. DNA in a sample may be attached to the flow cell and then synthesized and sequenced.

The barcode sequence or index sequence for sequencing may be used to multiplex DNA in multiple samples or DNA libraries obtained therefrom. Indexing DNA in samples or DNA libraries obtained therefrom by the barcode sequence or index sequence allows multiple samples or DNA libraries obtained therefrom to be pooled and sequenced simultaneously. The above-described indexing may be applied as single indexing or dual indexing technique.

The composition for kinship identification in Korean according to the present invention, which comprises an agent for amplifying or detecting an SNP located at 101 base position in at least one sequence, or all sequences from the group consisting of nucleotide sequences set forth as SEQ ID: 1 to SEQ ID NO: 918 and/or SEQ ID NO: 919 to SEQ ID NO: 1400, may be used in sequencing platforms known in the art, for example, including but not limited to, Illumina®, Thermo Fisher Scientific Ion Torrent™, Helicos Biosciences tSMS (true Single Molecule Sequencing), PacBio SMRT™ (Single-molecule real time), GridION™, and MinION™.

The term “first-degree relative” as used herein refers to any one of a subject's parent, child, brother, sister, and siblings. The term “first-degree relative” as used herein may be abbreviated as first-d-r.

The term “second-degree relative” as used herein refers to any one of a subject's maternal aunts, uncles, paternal aunts, grandparents, half-brothers, half-sisters, and half-siblings. The term “second-degree relative” as used herein may be abbreviated as second-d-r.

The term “third-degree relative” as used herein refers to a subject's first cousins. The term “third-degree relative” as used herein may be abbreviated as third-d-r.

In an embodiment of the present invention, the kinship is any one of a subject's parent, child, brother, sister, and sibling. That is, the kinship is any one of the subject's parent, child, brother, sister, and sibling. By using the composition for kinship identification in Korean according to the present invention, it is possible to distinguish a first-d-r individual that is in any one of parent, child, brother, sister, and sibling relationships with a subject, from non-first-d-r individuals. That is, the composition according to the present invention may distinguish a person who is in any one of parent, child, brother, sister, and sibling relationships with a subject, from unrelated persons.

In another embodiment of the present invention, the kinship is any one of a subject's brother, sister, and sibling. By using the composition for kinship identification in Korean according to the present invention, it is possible to distinguish a first-d-r individual that is in any one of brother, sister, and sibling relationships with a subject, from non-first-d-r individuals. That is, the composition according to the present invention can distinguish a person who is in any one of brother, sister, and sibling relationships with a subject, from unrelated persons, even when there is no parent's DNA information available.

In another embodiment of the present invention, the kinship is a relationship within 2-chon relatives based on the Korean kinship system, namely, parent-child relationship, monozygotic twin relationship, or full-sibling relationship. The composition for kinship identification in Korean according to the present invention may be used to distinguish individuals who are within 2-chon relationships, from individuals who are not within 2-chon relationships.

In an embodiment of the present invention, the composition for kinship identification in Korean, comprising an agent for amplifying or detecting an SNP located at position 101 in at least one sequence from among nucleotide sequences set forth in SEQ ID NO: 1 to SEQ ID NO: 918, can distinguish a person who is in any one of parent, child, brother, sister, and sibling relationships with a subject, from those who are not in any of the aforementioned relationships.

In another embodiment of the present invention, the composition for kinship identification in Korean, comprising an agent for amplifying or detecting an SNP located at position 101 in at least one sequence from among nucleotide sequences set forth in SEQ ID NO: 919 to SEQ ID NO: 1400, can distinguish a person who is in any one of parent, child, brother, sister, and sibling relationships with a subject, from those who are not in any of the aforementioned relationships.

In yet another embodiment of the present invention, the composition for kinship identification in Korean, comprising an agent for amplifying or detecting an SNP located at position 101 in at least one sequence from among nucleotide sequences set forth in SEQ ID NO: 1 to SEQ ID NO: 1400, can distinguish a person who is in any one of parent, child, brother, sister, and sibling relationships with a subject, from those who are not in any of the aforementioned relationships.

According to another aspect of the present invention, the present invention provides a marker composition for kinship identification in Korean, the marker composition comprising:
1) a polynucleotide comprising at least 10 consecutive nucleotides including an SNP located at position 101 in at least one sequence selected from the group consisting of nucleotide sequences set forth as SEQ ID NO: 1 to SEQ ID NO: 918, or a polynucleotide complementary thereto;
2) a polynucleotide comprising at least 10 consecutive nucleotides including an SNP located at position 101 in at least one sequence selected from the group consisting of nucleotide sequences set forth as SEQ ID NO: 919 to SEQ ID NO: 1400, or a polynucleotide complementary thereto; or
3) a polynucleotide comprising at least 10 consecutive nucleotides including an SNP located at position 101 in at least one sequence selected from the group consisting of nucleotide sequences set forth as SEQ ID NO: 1 to SEQ ID NO: 1400, or a polynucleotide complementary thereto.

In an embodiment of the present invention, the marker composition comprises a polynucleotide comprising at least 10 consecutive nucleotides including an SNP located at position 101 in any of sequences selected from the group consisting of nucleotide sequences set forth as SEQ ID NO: 1 to SEQ ID NO: 918, or comprises a polynucleotide complementary thereto.

In an embodiment of the present invention, the marker composition comprises a polynucleotide comprising at least 10 consecutive nucleotides including an SNP located at position 101 in at least 100, at least 200, at least 300, at least 400, at least 500, at least 600, at least 700, at least 800, at least 900, or 918 sequences selected from the group consisting of nucleotide sequences set forth as SEQ ID NO: 1 to SEQ ID NO: 918, or comprises a polynucleotide complementary thereto.

In another embodiment of the present invention, the marker composition comprises a polynucleotide comprising at least 10 consecutive nucleotides including an SNP located at position 101 in any of sequences selected from the group consisting of nucleotide sequences set forth as SEQ ID NO: 919 to SEQ ID NO: 1400, or comprises a polynucleotide complementary thereto.

In an embodiment of the present invention, the marker composition comprises a polynucleotide comprising at least 10 consecutive nucleotides including an SNP located at position 101 in at least 50, at least 100, at least 150, at least 200, at least 250, at least 300, at least 350, at least 400, at least 450, or 482 sequences selected from the group consisting of nucleotide sequences set forth as SEQ ID NO: 919 to SEQ ID NO: 1400, or comprises a polynucleotide complementary thereto.

In yet another embodiment of the present invention, the marker composition comprises a polynucleotide comprising at least 10 consecutive nucleotides including an SNP located at 101 base position in any of sequences selected from the group consisting of nucleotide sequences set forth as SEQ ID NO: 1 to SEQ ID NO: 1400, or comprises a polynucleotide complementary thereto.

In an embodiment of the present invention, the marker composition comprises a polynucleotide comprising at least 10 consecutive nucleotides including an SNP located at 101 base position in at least 1, at least 10, at least 100, at least 200, at least 300, at least 400, at least 500, at least 600, at least 700, at least 800, at least 900, at least 1,000, at least 1,100, at least 1,200, at least 1,300, or 1,400 sequences selected from the group consisting of nucleotide sequences set forth as SEQ ID NO: 1 to SEQ ID NO: 1400, or comprises a polynucleotide complementary thereto.

The marker composition for kinship identification in Korean of the present invention comprises an SNP that is amplified or detected by an agent comprised in the above-described composition for kinship identification in Korean according to another aspect, any description that may be redundant will be omitted for clarity of this specification.

According to another aspect of the present invention, the present invention provides a kit for kinship identification in Korean, comprising the above-described composition for kinship identification in Korean.

In an embodiment of the present invention, the kit is a microarray. The kit may be used for sequencing.

The microarray may have a polynucleotide comprising the above-described SNP, or a polynucleotide complementary thereto, arranged at high density in a specific area of substrate surface. In particular, in the microarray, a polynucleotide comprising at least 10 consecutive nucleotide sequences containing an SNP located at 101 base position in at least one sequence selected from the group consisting of nucleotide sequences set forth as of SEQ ID NO: 1 to SEQ ID NO: 918 and/or SEQ ID NO: 919 to SEQ ID NO: 1400, or a nucleotide sequence complementary thereto, may be arranged at high density.

The flow cell may be a glass with a well having a nano-scale diameter. In the well, a DNA probe capable of detecting or amplifying the above-described SNP in the composition for kinship identification in Korean is comprised, or a primer that hybridizes to the above-described SNP is attached.

The kit may further comprise the reagents required for detection or amplification of the above-described SNP, for example, but not limited to, dNTP, DNA polymerases such as Taq DNA polymerase and ϕ29 DNA polymerase, distilled water, Tris-HCl, KCl (potassium chloride), MgCl₂ (magnesium chloride), and the like.

In addition, the kit may further comprise the equipment required for detection or amplification of the above-described SNP, for example, but not limited to, a thermocycler, a PCR system, a sequencer, and the like.

Furthermore, the primer attached to the well of the kit hybridizes with the SNP in a sample or an adaptor connected to the SNP, and a kinship may be identified from the results of the hybridization. The kit may further comprise the reagents required for hybridization of primers and the SNP or adaptors linked to the SNP, examples being but not limited to T4 DNA polymerase for repairing DNA fragments containing SNPs to blunt ends, Klenow fragments, T4 polynucleotide kinases, dATP for A-tailing, DNA ligase for ligation of adaptors to DNA fragments including SNPs, an index primer capable of amplifying DNA fragments ligated with adaptors, and the like.

The kit for kinship identification in Korean comprises the above-described composition for kinship identification in Korean according to another aspect, and thus any description that may be redundant will be omitted for clarity of this specification.

According to another aspect of the present invention, the present invention provides a method of kinship identification in Korean, the method comprising:
(1) identifying a nucleotide of an SNP located at position 101 in at least one sequence selected from the group consisting of nucleotide sequences set forth as SEQ ID NO: 1 to SEQ ID NO: 918, in samples isolated from two or more individuals whose kinship is to be identified; or
(2) identifying a nucleotide of an SNP located at position 101 in at least one sequence selected from the group consisting of nucleotide sequences set forth as SEQ ID NO: 919 to SEQ ID NO: 1400, in samples isolated from two or more individuals whose kinship is to be identified.

The method of determining the type of base, that is, the identity of nucleotide at the SNP site may be any method known in the art, examples including, but not limited to any known sequencing method, Sanger sequencing, Maxam-Gilbert Sequencing, capillary electrophoresis and fragment analysis, and next-generation sequencing (NGS). Preferably, the identity of nucleotide at the SNP site is determined by NGS, and more information can be found in literature (Metzker M L. Sequencing technologies—the next generation. Nat Rev Genet. 2010 January; 11(1l):31-46; Børsting C, Morling N. Next generation sequencing and its applications in forensic genetics. Forensic Sci Int Genet. 2015 September; 18:78-89; and Alvarez-Cubero M J et al. Next generation sequencing: an application in forensic sciences?Ann Hum Biol. 2017 November; 44(7):581-592).

In addition, the type of nucleotide at the SNP site may be determined by hybridizing a probe or primer complementary to an SNP flanking sequence of 10 bp to 25 bp, located between 20 bp upstream and 20 bp downstream from the SNP site and including the SNP, to a DNA fragment including the target SNP, and analyzing the results of the hybridization.

In an embodiment of the present invention, the above method further comprises:

In case of (1) above, identifying the SNP located at position 101 in at least one sequence selected from the group consisting of nucleotide sequences set forth as SEQ ID NO: 919 to SEQ ID NO: 1400; or
in case of (2) above, identifying the nucleotide of the SNP located at position 101 in at least one sequence selected from the group consisting of nucleotide sequences set forth as SEQ ID NO: 1 to SEQ ID NO: 918.

In an embodiment of the present invention, the method comprises identifying the SNP located at position 101 in at least 100, at least 200, at least 300, at least 400, at least 500, at least 600, at least 700, at least 800, at least 900, or 918 sequences selected from the group consisting of nucleotide sequences set forth as SEQ ID NO: 1 to SEQ ID NO: 918, in a sample isolated from the two or more individuals whose kinship is to be identified.

In another embodiment of the present invention, the method comprises identifying the SNP located at position 101 in at least 50, at least 100, at least 150, at least 200, at least 250, at least 300, at least 350, at least 400, at least 450, or 482 sequences selected from the group consisting of nucleotide sequences set forth as SEQ ID NO: 919 to SEQ ID NO: 1400, in a sample isolated from the two or more individuals whose kinship is to be identified.

In another embodiment of the present invention, the method comprises identifying the SNP located at position 101 in at least 1, at least 10, at least 100, at least 200, at least 300, at least 400, at least 500, at least 600, at least 700, at least 800, at least 900, at least 1000, at least 1100, at least 1200, at least 1300, or 1400 sequences selected from the group consisting of nucleotide sequences set forth as SEQ ID NO: 1 to SEQ ID NO: 1400, in a sample isolated from the two or more individuals whose kinship is to be identified.

In another embodiment of the present invention, the method comprises identifying the SNP located at position 101 in nucleotide sequences set forth as SEQ ID NO: 1 to SEQ ID NO: 1400 in samples isolated from the two or more individuals whose kinship is to be identified.

In an embodiment of the present invention, the kinship is any one of a subject's parent, child, brother, sister, and sibling. That is, by using the present invention, it is possible to distinguish a person who is in any one of parent, child, brother, sister, and sibling relationships with a subject, from unrelated persons.

In an embodiment of the present invention, the kinship is any one of a subject's brother, sister, and sibling. That is, by using the present invention, it is possible to distinguish a person who is in any one of brother, sister, and sibling relationships with a subject, from unrelated persons. The method according to the present invention can distinguish a person who is in any one of brother, sister, and sibling relationships from unrelated persons even when there is no parental DNA information.

In an embodiment of the present invention, the identifying the SNP is amplifying or detecting the SNP by using a primer, a probe, or a mixture thereof.

In another embodiment of the present invention, the identifying the SNP is amplifying or detecting the SNP by using an oligomer or primer for sequencing.

In a specific embodiment of the present invention, the method further comprises, after the identifying the SNP, making pairwise comparison of each SNP in each sample.

In a specific embodiment of the present invention, the making pairwise comparison of each SNP in each sample comprises calculating a kinship coefficient or coefficient of relatedness, as known in the art.

In a specific embodiment of the present invention, the kinship coefficient or coefficient of relatedness may be calculated by IBS (identify by state) scoring, IBD (identical by descent) scoring, or likelihood ratio (LR). As for methods of measuring/calculating IBD, IBS, and/or LR from SNP data, and calculating therefrom a kinship coefficient or coefficient of relatedness, more information can be found in various literature (Frudakis T et al. A classifier for the SNP-based inference of ancestry. J Forensic Sci. 2003 July; 48(4):771-82. Erratum in: J Forensic Sci. 2004 September; 49(5):1145-6; Stevens E L et al. Inference of relationships in population data using identity-by-descent and identity-by-state. PLoS Genet. 2011 September; 7(9):e1002287; Browning B L, Browning S R. A fast, powerful method for detecting identity by descent. Am J Hum Genet. 2011 Feb. 11; 88(2):173-82; Zheng X et al. A high-performance computing toolset for relatedness and principal component analysis of SNP data. Bioinformatics. 2012 Dec. 15; 28(24):3326-8; and Browning B L, Browning S R. Improving the accuracy and efficiency of identity-by-descent detection in population data. Genetics. 2013 June; 194(2):459-71).

As described in one example of the present invention, kinship coefficient or coefficient of relatedness can be extracted from 918- and/or the 482-SNP panel of the present invention by using Somalier program disclosed in Pedersen et al. 2020 (Somalier: rapid relatedness estimation for cancer and germline studies using efficient genome sketches. Genome medicine, 12(1), 1-9). However, the method by which a kinship coefficient or coefficient of relatedness can be extracted is not limited thereto, and any method known in the art of obtaining a kinship coefficient or coefficient of relatedness from SNP data may be applied to the present invention.

In an specific embodiment, the making pairwise comparison for each SNP nucleotide in each sample includes the following steps:
(a) i) when all nucleotides of the SNP identified from two alleles in two samples being pairwise compared are identical, assigning an IBS (identity by state) score of 2 to the SNP, ii) when only one nucleotide of the SNP is identical between two alleles in two samples being pairwise compared, assigning an IBS score of 1 to the SNP, or iii) when all nucleotides of the SNP identified from two alleles in two samples being pairwise compared are different, assigning an IBS score of 0 to the SNP; and
(b) obtaining an average of the IBS scores of all the SNPs being compared pairwise. In an specific embodiment of the present invention, when the average of IBS score from the (b) obtaining an average is 0.300 to 0.700, there is provided information that the two individuals from which the two samples pairwise compared were isolated are in any one of kinship selected from the group consisting of parent, child, brother, sister, and sibling relationships; or
when the average IBS score obtained from the (b) obtaining an average is less than 0.300, there is provided information indicating that the two individuals from which the two samples pairwise compared were isolated are not in any one of kinship selected from the group consisting of parent, child, brother, sister, and sibling.

In another specific embodiment of the present invention, when the average IBS score is from 0.380 to 0.700, there is provided information that the two individuals from which the two samples pairwise compared were isolated are in any one of kinship selected from the group consisting of parent, child, brother, sister, and sibling relationships; or
when the average IBS score is less than 0.380, there is provided information that the two individuals from which the two samples pairwise compared were isolated are not in any of kinship selected from the group consisting of parent, child, brother, sister, and sibling relationships.

The subject whose kinship is to be identified may be two individuals. The subject whose kinship is to be identified may be two or more individuals.

In an embodiment of the present invention, the subject whose kinship is to be identified may be at least 2 individuals, at least 10 individuals, at least 20 individuals, at least 40 individuals, at least 50 individuals, or at least 100 individuals. In another embodiment of the present invention, the subject whose kinship is to be identified may be, but is not limited to, 2 individuals to 10 individuals, 2 individuals to 20 individuals, 2 individuals to 40 individuals, 2 individuals to 50 individuals, or 2 individuals to 100 individuals. Here, those skilled in the art would appreciate that citing these aforementioned numbers is not to limit the number of individuals whose kinship is to be identified. The subject whose kinship is to be identified comprises missing persons, unknown corpses, person with unconfirmed identity with no living parents, war casualties, the dead and wounded in massive disaster events, incident victims, human remains, the surviving family members thereof, or potential surviving family members thereof. However, the subject whose kinship is to be identified may include a far greater number of individuals than those aforementioned if need be.

In addition, the number of individuals whose kinship is to be identified may be determined based on factors such as the performance of the sequencing platform used, the size of the final output (Gb, giga base) of sequencing, the quality, purity, and DNA yield of the samples being analyzed, and the like. The final output (Gb) of sequencing may be determined according to the number of amplicons, and the size and coverage of amplicons. For example, if the final output of sequencing is about 1 Gb, the subject whose kinship is to be identified may be at least 10 individuals, but is not necessarily limited thereto. As an another example, if the purity and yield of DNA isolated from a blood sample from a particular subject are sufficiently high, the user may select a smaller final output of sequencing to thereby increase the number of individuals whose kinship is to be identified with respect to that particular subject and may proceed the sequencing by selecting indexing combination of individuals. The user ultimately can adjust sequencing quality and adjust the possible number of individuals that can be analyzed by selecting the size of final output of sequencing, selecting the sequencing equipment with appropriate performance, or adjusting the sequencing running time in accordance with the purpose of kinship identification, or the purity and yield of a sample to be analyzed.

The subject is a human. The subject is a Korean individual. The subject may be a native or resident of the Korean peninsula, or may be a Korean descent who does not reside in the Korean peninsula. The subject may be but is not limited to, a missing person, an unknown corpse, a person with unconfirmed identity with no living parents, war casualties, the dead and wounded in massive disaster events, an incident victim, human remains, the surviving family members thereof, potential surviving family members, or the like.

The sample may be a cell, a tissue, an organ, or bodily fluid isolated from the subject. The sample may be a naturally existing sample, a human remain sample from old human remains, a sample obtained from a forensic site, an archeological sample such as mummified tissues, a refrigerated or frozen sample, a sample fixed by fixer such as formalin, a sample embedded in paraffin, or a sample treated with preservatives. The sample includes but is not necessarily limited to, oral swab, a vaginal swab, a rectal swab, saliva, sweat, urine, feces, mucus, semen, blood, plasma, serum, bloodstain, cerebrospinal fluid, ascites, amniotic fluid, tears, discharges, bones, and the like.

In another embodiment of the present invention, the method of kinship identification in Korean comprises the following:
1) isolating DNA from at least two Korean individuals whose kinship is to be identified;
2) identifying an SNP located at position 101 of nucleotide sequences set forth as SEQ ID NO: 1 to SEQ ID NO: 918 in the DNA;
3) inputting data regarding the identity of nucleotide at the SNP to a computer-readable medium in which information of the SNP located at position 101 in the nucleotide sequences set forth as SEQ ID NO: 1 to SEQ ID NO: 918;
4) calculating an average IBS score between two Korean individuals by using the computer-readable medium; and
5) providing information that the two Korean individuals have a kinship that is any one of parent, child, brother, sister, and sibling if the average IBS score is between 0.3000 and 0.7000, or providing information that the two Korean individuals do not have a kinship that is any one of parent, child, brother, sister, and sibling if the average IBS score is less than 0.300.

The computer-readable medium may be those available online, such as PLINK (zzz.bwh.harvard.edu/plink/) (Purcell et al. 2007), KING (Kinship-based Inference for Gwas) (https://www.kingrelatedness.com/) but is not necessarily limited thereto and rather, any medium that is capable of calculating kinship coefficients or coefficients of relatedness from inputted SNP panel information and DNA sequencing data may be used without limitation. In addition, the computer-readable medium may be Somalier program (Pedersen et al. 2020).

The SNP information input to the computer-readable medium may include but are not limited to the chromosome number on which the SNP is located, the position on the chromosome, and SNP's unique identification number such as dbSNP rs number, reference alleles that appear on a reference genome at the SNP position, alternative alleles that appear at the SNP position, and the like.

As used herein, the term “reference allele (ref. allele)” refers to a base found at a corresponding locus in the reference genome. The reference allele does not always mean that it is the major allele.

As used herein, the term “alternative allele (alt. allele)” refers to any base other than the base in the reference allele defined above, that is found at the given locus.

Based on information of 918 and/or 482 SNPs inputted, the computer-readable medium makes pairwise comparison of sequencing data of DNA isolated from at least two Korean individuals, that is, makes pairwise comparison of nucleotides at a specific SNP position, to calculate IBS (identity by state), IBD (identical by descent), or likelihood ratio (LR) values. An average value of the calculated IBS, IBD or LR values is taken and a kinship coefficients or coefficient of relatedness is extracted therefrom.

In another specific embodiment of the present invention, the method of kinship identification in Korean comprising the 2) identifying an SNP located at position 101 of nucleotide sequence(s) selected from the group consisting of SEQ ID NO: 1 to SEQ ID NO: 918 in the DNA isolated from at least two Korean individuals whose kinship is to be identified, after the identifying the SNP, comprises making pairwise comparison of each SNP nucleotide in each sample, and the making pairwise comparison of each SNP nucleotide in each sample comprises as follow:
(a) i) when all nucleotides of the SNP identified from two alleles in two samples being pairwise compared are identical, assigning an IBS score of 2 to the SNP, ii) if only one nucleotide of the SNP is identical between two alleles in two samples being pairwise compared, assigning an IBS score of 1 to the SNP, or iii) if all nucleotides of the SNP identified from two alleles in two samples being pairwise compared are different, assigning an IBS score of 0 to the SNP; and
(b) obtaining an average value of IBS scores of all the SNPs compared pairwise; wherein when the average IBS score obtained from the (b) obtaining an average value is 0.310 to 0.700, providing information that there is a possibility that the two individuals from which the two samples pairwise compared were isolated are in any one of parent, child, brother, sister, and sibling relationships; or
wherein when the average IBS score obtained from the (b) obtaining an average value is less than 0.310, providing information that there is a possibility that the two individuals from which the two samples pairwise compared were isolated are not in any of parent, child, brother, sister, and sibling relationships.

In another specific embodiment of the present invention, when the average IBS score is from 0.340 to 0.700, there is provided information that there is a possibility that the two individuals from which the two samples pairwise compared were isolated are in any one of parent, child, brother, sister, and sibling relationships; or
when the average IBS score is less than 0.340, there is provided information that there is a possibility that the two individuals from which the two samples pairwise compared were isolated are not in any of parent, child, brother, sister, and sibling relationships.

In another embodiment of the present invention, the method of kinship identification in Korean comprising the following:
1) isolating DNA from at least two Korean individuals whose kinship is to be identified;
2) identifying an SNP located at position 101 of nucleotide sequences set forth as SEQ ID NO: 919 to SEQ ID NO: 1400 in the DNA;
3) inputting data regarding nucleotide types of the SNP to a computer-readable medium having input of information of the SNP located at position 101 in the nucleotide sequences set forth as SEQ ID NO: 1 to SEQ ID NO: 918;
4) calculating an average IBS score between two Korean individuals by using the computer-readable medium; and
5) providing information that there is possibility that the two Korean individuals are in any one of parent, child, brother, sister, and sibling relationships if the average IBS score is between 0.310 and 0.700, or providing information that there is possibility that the two Korean individuals are not in any of parent, child, brother, sister, and sibling relationships if the average IBS score is less than 0.310.

The method of kinship identification in Korean according to the present invention will be described step-by-step.

The Step of Isolating Nucleic Acids from a Sample Isolated from a Subject

The method of kinship identification in Korean may further comprise a step of isolating nucleic acids from a sample isolated from a subject. As a method of isolating nucleic acids from a sample, any method well-known in the art, examples being but not necessarily limited to: an extraction method using acid guanidinium thiocyanate-phenol-chloroform, a method using PCI (phenol-chloroform-isoamyl alcohol) solution, and the like, and specific details of such methods are disclosed in Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985). In order to isolate nucleic acids from a sample isolated from a subject, a commercially available nucleic acid extraction kit, nucleic acid purification kit, and the like, may be used. The nucleic acids may be made to additionally or repeatedly undergo a purification or quality control process.

In particular, from the sample isolated from the subject, genomic DNA is extracted, purified, and subjected to quality control (QC). Commercially available spectrometers or fluorometers, such as NanoDrop and Qubit by Thermo Scientific, may be used to check for DNA quality such as confirming whether the extracted DNA molecules are double-stranded DNA and at a concentration appropriate for later sequencing, and checking DNA purity by investigating A260/280 ratio and A260/230 ratio, checking DNA degradation through electrophoresis, and the like. DNA having passed QC standards appropriate for sequencing are used to construct libraries at a later stage. Any and all methods of DNA extraction, purification, and QC known in the art may be used in the present invention without limitations.

Step of Identifying the Nucleotide of SNP Located at 101 Base Position in at Least One Sequence Selected from the Group Consisting of Nucleotide Sequences Set Forth in SEQ ID NO: 1 to SEQ ID NO: 918; SEQ ID NO: 919 to SEQ ID NO: 1400; or SEQ ID NO: 1 to SEQ ID NO: 1400

The step of identifying the SNP may utilize without limitations, any DNA amplification method, DNA detection method, or sequencing method known in the art. Among the sequencing methods, it is preferable to use the next-generation sequencing (NGS) method.

Hereinbelow, the sequencing method for identification of the SNP will be described step-by-step.

### (1) DNA Fragmentation

DNA having passed the above-described QC standards are subject to fragmentation. DNA fragmentation may be performed by physical methods or chemical methods using enzymes known in the art. Mechanical methods involving breakage of phosphodiester linkages of DNA molecules by applying shear force include acoustic shearing, sonication, nebulization, hydrodynamic shearing, and the like. Also, commercially available Adaptive Focused Acoustic® technology provided by Covaris, Bioruptor® DNA shearing technology provided by Diagenode, HydroShear Plus Hydrodynamic DNA shearing instrument provided by DigiLab, and the like may also be utilized without necessarily being limited thereto.

DNA fragmentation using enzymatic cleavage includes methods of generating a double strand break by creating a nick in each strand or cutting two strands of DNA with endonuclease and nicking enzymes, and the like. DNA size can be adjusted by adjusting enzymatic digestion time.

In addition to the above-described methods of DNA fragmentation using physical shearing and enzymatic digestion, DNA fragmentation methods using transposon may also be utilized (Adey A. et al., Genome Biol. 2010; 11(12):R119).

After DNA fragmentation, DNA having a size of 300 bp to 500 bp are selected by electrophoresis. After DNA fragmentation, QC may be additionally conducted.

### (2) End Repair and A-Tailing

Damaged ends of the fragmented DNAs obtained above, that is, 5′ overhangs and 3′ overhangs, are repaired into blunt ends by using T4 DNA polymerase, Klenow fragment, or the like. Next, the blunt ends are phosphorylated at 5′ ends by T4 polynucleotide kinase, and adenylated, that is, A-tailed at 3′ ends by Klenow fragment or Taq DNA polymerase. A-tailing is necessary for later linking an adaptor to DNA.

### (3) Adaptor Ligation

Adaptors are ligated to both ends of the DNA fragments obtained above to allow oligonucleotides on a flow cell to be able to recognize the adaptors for sequencing at a later stage. The adaptor is a pair of oligonucleotides to which primers anneal for DNA amplification at a later stage. To remove adaptors unincorporated in DNA fragments and adaptor dimers, an additional clean-up process may be performed.

### (4) Construction of Libraries and Amplification

In order to construct libraries of good quality and high yield, DNA should be amplified using from the smallest possible amount to a large amount of DNA, and the libraries prepared should be able to produce high yield even when using a small amount of DNA. Since PCR errors later give rise to errors in sequencing, it is preferable to run a smaller number of PCR cycles, and in order to perform PCR with high uniformity regardless of GC content and AT content in sequence, polymerases with high activity and fidelity should be used. Library amplification may be performed using an index primer that binds to an adapter, and the index primer may be single or dual.

To generate libraries of the DNA ligated with adaptors above, library amplification may be performed with or without the use of PCR. DNA library amplification not involving the use of PCR is often used to generate libraries with higher GC content and AT content, and since this requires a large quantity of DNA, generating libraries from degraded nucleic acids or a small amount of sample, amplification is performed with the use of PCR. Common PCR can introduce GC bias, which hinders de novo sequencing and SNP discovery.

Therefore, the present inventors used a DNA amplification method based on RCA (rolling circular amplicon) in order to reduce such PCR errors. However, any DNA amplification method with low amplification error rate known in the art may be used without limitations.

In particular, RCA is a process of unidirectional nucleic acid replication that can rapidly synthesize circular molecules of DNA or RNA, wherein the replication is initiated a nicked strand of the double-stranded circular DNA molecule. Using the unnicked strand as a template and separating the nicked single-stranded DNA as a result of replication, continued DNA synthesis can produce a long linear single-stranded continuous DNA concatemer.

For RCA-based PCR, the linear double-stranded DNA ligated with adaptors prepared above may be additionally amplified by PCR. After PCR amplification, additional QC may be run.

In one example of the present invention, the above-described RCA-based DNBSEQ-T7, which is a DNA nano ball (DNB) sequencing set provided by MGI, was used, but those skilled in the art would appreciate that the method is not necessarily limited thereto, and any commercially available sequencing platform may be used, examples being Illumine®, Nova-Seq, Thermo Fisher Scientific Ion Torrent™, Helicos Biosciences tSMS (true Single Molecule Sequencing), PacBio SMRT™ (Single-molecule real time), GridION™, and MinION™.

The PCR products obtained above, which are linear double-stranded DNA molecules, are heat-denatured into linear single-stranded DNA molecules and then connected by DNA ligase to form circular single-stranded DNA molecules. QC may be additionally run on the circular single-stranded DNA molecules. After adding primers for DNA nano balls (DNB) to the circular single-stranded DNA molecules obtained following the manufacturer's instructions, RCA reaction was performed. The obtained DNB was pooled and DNB sequencing was performed using high density patterned nanoarray flow cells by MGI Tech, which allow only one DNB bound per active site.

### (4) Sequencing

Sequencing (genome decoding) may be performed by methods known in the art and may be performed through main steps as shown in the WGS (whole genome sequencing) analysis pipeline diagram illustrated in FIG. 9.

### 1) Sequence Generation

As a result of sequencing, a nucleotide sequence is identified from a DNA fragment and generated in a read unit. For each read, a Phred quality score, which is a per-base quality index, is generated together, and this is stored in a FastQ file with the nucleotide sequence information.

As used herein, the term “Phred quality score” refers to a quality index indicating the reliability of each base, and is a value representing the estimated probability of error per base. Phred quality scores are used as a metric to determine how accurately each sequence is read from sequencing data. The higher the score, the more likely that the base calling is accurate.

Phred quality scores (Q) can be calculated by Equation 1 below.

$${}^{*} 193 \qquad [ Equation 1 ]$$

$$Q = - 10 log ( error probability )$$

For example, if the probability of error per base is 1/1000 and 1/10000, the Phred quality scores (Q) are Q30 and Q40, respectively.

The term “read” as used herein refers to information of base pairs of analysis amount generated from a DNA or cDNA fragment included in a sequencing library. The read includes data, sequence, or base sequence fragment output as a result of sequencing.

The term “FastQ file” as used herein refers to a file containing identification number and sequence of each read, and quality index corresponding for each read.

Next, the quality of FastQ file is evaluated. Programs capable of evaluating the quality of FastQ files include FastQC, PRINSEQ, Trimmomatic and the like.

In one example of the present invention, FastQC program was used to check per-base quality and check for any defect or issue. In addition, using Cutadapt program (DOI:10.14806/ej.17.1.200), adaptor sequences were removed from the sequencing reads, and using FastP, FastQ data was preprocessed to filter quality. Before proceeding to the sequence alignment process, per-base quality can be further checked using FastQC program.

### 2) Sequence Alignment

The sequence of generated reads and the sequence of a human reference genome are aligned. For the human reference genome, GRCh 38 or GRCh 37 may be used, and this is disclosed in public database (GRCh 38, USCS version Hg19, https://hgdownload.cse.ucsc.edu/goldenPath/hg19/bigZips/; and GRCh 37, USCS version Hg38, https://hgdownload.cse.ucsc.edu/goldenPath/hg38/bigZips/). Through sequence alignment, the original position of a corresponding DNA fragment in the genome can be estimated. Using BWA-MEM program (Heng Li, 2013), the reads are aligned based on the similarity with the reference genome. The results are stored as BAM file.

### 3) Removal of Duplicate Sequences

After sequence alignment, sequence reads determined as PCR duplicates are marked by tags using Picard program. The results are stored as BAM file.

### 4) Base Quality Score Recalibration (BQSR)

Using GATK4 program, error patterns in each base quality score calculated by the sequencing device were detected and corrected using machine learning techniques. The results are stored as BAM file.

### 5) Quality Evaluation

After sequence alignment, quality evaluation of the BAM files is performed to indirectly determine authenticity of individuals being sequenced and any defect in sampling and library construction. Quality evaluation includes depth of coverage, mean depth of coverage, duplication rate, and the like. Depth of coverage refers to the number of reads mapped at a target point. Duplication rate refers to the percentage of PCR duplicate reads, which are copies of the same DNA fragment that occur in the PCR process during sequencing.

### 6) Variant Calling

Variants were called by detecting variations between the reference genome and a generated sequence. When BAM files are input to GTK4 program, detected variations are stored in VCF file format. The term “VCF (variant cell format) file” as used herein refers to a standardized file format used for representing variations determined as differing from the reference genome and their frequencies.

A VCF file contains information such as the chromosome on which the variation is located, the position of the variation on the chromosome, the unique identification number of the variation such as dbSNP rs number, the base (REF) that appears at the variation position in the reference genome, alternative alleles (ALT) (that is, variants), a quality score, a filter name indicated in the variation, additional information about the variation, the format of a sample genotype, and the like.

### (5) Extraction of Kinship Coefficient or Coefficient or Relatedness

The present inventors calculated an average IBS score between two individuals from variations of 918 and/or 482 SNP markers from a BAM file previously extracted using Somalier program (Pedersen et al. 2020).

In an embodiment of the present invention, as a result of deriving an average IBS score between two individuals from variations of the 918 SNP markers, if the average IBS score between two individuals whose kinship is to be identified is located in a buffer region of 0.300 to 0.700, information that these two individuals in a first-degree relationship with each other is provided, or if the IBS value between two individuals is less than 0.300, information that these two individuals are not in a first-degree relationship with each other is provided.

In another embodiment of the present invention, as a result of deriving an average IBS score between two individuals from variations of the 918 SNP markers, if the average IBS score between two individuals whose kinship is to be identified is located in a buffer region of 0.380 to 0.700, information that these two individuals are in a first-degree relationship with each other is provided, or if the IBS value between two individuals is less than 0.380, information that these two individuals are not in a first-degree relationship with each other is provided.

In yet another embodiment of the present invention, as a result of deriving an average IBS score between two individuals from variations of the 918 SNP markers, if the average IBS score between two individuals whose kinship is to be identified is located in a buffer region of 0.382 to 0.574, information that these two individuals are in a first-degree relationship with each other is provided; if the average IBS score between two individuals is in a buffer region of 0.166 to 0.299, information that these two individuals are in a second-degree relationship with each other is provided; and if the average IBS score between two individuals is in a buffer region of −0.193 to 0.144, information that these two individuals are unrelated or not in any familial relationship is provided.

In yet another embodiment of the present invention, as a result of deriving an average IBS score between two individuals from variations of the 918 SNP markers, if the average IBS score between two individuals whose kinship is to be identified is located in a buffer region of 0.413 to 0.635, information that these two individuals are first-degree relatives to each other is provided; if the average IBS score between two individuals is in a buffer region of 0.087 to 0.378, information that these two individuals are second-degree relatives to each other is provided; and if the average IBS score between two individuals is in a region of 0.273 or less (including negative IBS values), information that these two individuals are unrelated or not in any familial relationship is provided.

When using the 918 SNP markers according to the present invention, even when a smaller number of SNPs is checked compared to the prior art methods, and even when no parent information is available, since the buffer regions in which the average IBS score of two individuals in a first-degree relationship is located and the buffer region in which the average IBS score of two individuals in a second-degree relationship is located are clearly separated from each other, that is, the two buffer regions do not overlap but are distinguished from each other, it is possible to clearly distinguish, with respect to a subject, individuals who are in a first-d-r relationship as one of parent, child, brother, sister, and sibling, from individuals who are not in any first-d-r relationship.

It is considered that the use of the 482 SNP markers as shown in SEQ ID NO: 919 to SEQ ID NO: 1400 in addition to the above-described 918 SNP markers as shown in SEQ ID NO: 1 to SEQ ID NO: 918 would increase the accuracy of kinship identification analysis.

In an embodiment of the present invention, as a result of deriving an average IBS score between two individuals from variations of the 482 SNP markers, if the average IBS score between two individuals whose kinship is to be identified is located in a buffer region of 0.310 to 0.700, information that there is possibility of these two individuals being in a first-d-r relationship is provided, and if the average IBS score between two individuals is less than 0.310, information that there is possibility of these two individuals not being in a first-d-r relationship is provided.

In another embodiment of the present invention, as a result of deriving an average IBS score between two individuals from variations of the 482 SNP markers, if the average IBS score between two individuals whose kinship is to be identified is located in a buffer region of 0.340 to 0.700, information that there is possibility of these two individuals being in a first-d-r relationship is provided, and if the average IBS score between two individuals is less than 0.340, information that there is possibility of these two individuals not being in a first-d-r relationship is provided.

In yet another embodiment of the present invention, as a result of deriving an average IBS score between two individuals from variations of the 482 SNP markers, if the average IBS score between two individuals whose kinship is to be identified is located in a buffer region of 0.340 to 0.620, information that there is possibility of these two individuals being in a first-d-r relationship with each other is provided; if the average IBS score between two individuals is in a buffer region of 0.175 to 0.309, information that there is possibility of these two individuals being in a second-d-r relationship with each other is provided; and if the average IBS score between two individuals is in a buffer region of −0.277 to 0.175, information that there is possibility of these two individuals being unrelated or not in any familial relationship is provided.

In yet another embodiment of the present invention, as a result of deriving an average IBS score between two individuals from variations of the 482 SNP markers, if the average IBS score between two individuals whose kinship is to be identified is located in a buffer region of 0.378 to 0.667, information that there is possibility of these two individuals being in a first-d-r relationship with each other is provided; if the average IBS score between two individuals is in a buffer region of 0.087 to 0.414, information that there is possibility of these two individuals being in a second-d-r relationship with each other is provided; and if the average IBS score between two individuals is in a region of 0.270 or less (including negative IBS values), information that there is possibility of these two individuals being unrelated or not in any familial relationship is provided.

When using the 482 SNP markers according to the present invention, the buffer region in which an average IBS score of two individuals in a first-d-r relationship is located partially overlaps with the buffer region in which an average IBS score of two individuals in a second-d-r relationship is located. Therefore, the use of the 482 SNP markers may be slightly limited in distinguishing individuals in first-d-r relationships with a subject, from individuals who are in second-d-r relationships with the subject, but can be useful in distinguishing individuals who are in first-d-r relationships with the subject from those who are not in any first-d-r relationships. In the field of forensic science, any evidence indicating that the possibility of two individuals being in a familial relationship cannot be excluded holds significance. Since the 482 SNP markers according to the present invention makes it possible to distinguish individuals in a first-d-r relationship from the unrelated, individuals possibly in a first-d-r relationship from the unrelated, or individuals highly likely in a first-d-r relationship from the unrelated by simple pairwise comparison of only 482 minimum number of SNPs, it is cost-effective and can reduce the time required for kinship identification, and accordingly, the 482 SNP markers according to the present invention are useful in the field of forensic science, legal medicine, or criminal investigation.

It is considered that use of the 918 SNP markers as shown in SEQ ID NO: 1 to SEQ ID NO: 918 in addition to the above-described 482 SNP markers as shown in SEQ ID NO: 919 to SEQ ID NO: 1400 would increase the accuracy of kinship identification analysis.

Since the method of kinship identification in Korean is enabled by using the above-described preparation for amplifying or detecting at least one of SNPs located at position 101 in nucleotide sequences set forth in SEQ ID NO: 1 to SEQ ID NO: 918 and/or SEQ ID NO: 919 to SEQ ID NO: 1400 according to another aspect of the present invention, any descriptions deemed redundant will be omitted in the interest of clarity of this application.

According to yet another aspect of the present invention, the present invention provides a method of discovering an SNP marker for kinship identification, the method comprising extracting, from the human genome database, SNPs characterized by at least one of the following features:
SNPs having a p value of 0.05 or more at Hardy-Weinberg equilibrium (HWE);
SNPs that is not present within a genomic region or within 100 kbp upstream or downstream of the genomic regions;
SNPs having a variant allele frequency of 0.3 to 0.7;
SNPs not present in linkage disequilibrium (LD); and
SNPs not present in repeated regions.

In an embodiment of the present invention, the SNP has a variant allele frequency of 0.4 to 0.6.

In an embodiment of the present invention, the human genome database may be a reference genome. In a specific embodiment of the present invention, the reference genome is a standard sequence that is completely sequenced and established in public database. The reference genome may be GRCh37/hg19 or GRCh38/hg38.

In another embodiment of the present invention, the human genome database is dbSNP (Single Nucleotide Polymorphism database) which is open database provided by NCBI (National Center for Biotechnology Information).

In another embodiment of the present invention, the human genome database may be a Korean reference genome. The Korean reference genome is a variome obtained from the whole genome sequence (WGS) of 397 Korean individuals (Y J et al., 2015), the WGS of 200 Korean individuals (Lee et al., 2017, sequencing studies in cardiovascular diseases) or from KoVariome, which is Korea National Standard Reference Variome database.

Those skilled in the art would appreciate that SNP panels for kinship identification specific to each population can be discovered by referring to the method of discovering SNP markers for kinship identification according to the present invention.

According to yet another embodiment of the present invention, the human genome database may be a reference genome of a specific population, including but not limited to, Europeans, Africans, African Americans, Asians, East Asians, other Asians, Latin Americans, and South Asians.

For example, the human genome database may be ALFA (Allele Frequency Aggregator), Japanese reference genome 8.3KJPN, global reference genome (1000G, 1000 Genomes Project), Estonian reference genome, reference genome of a birth cohort in the Bristol area of the UK (ALSPAC, the Avon Longitudinal Study of Parents and Children), British twin reference genome TwinsUK, Korean reference genome KOREAN, HGDP Stanford (The Human Genome Diversity Project), HapMap project, GoNL (Genome of the Netherlands), Swedish reference genome NorthernSweden, SGDP PRJ (Simons Genome Diversity Project), QGP (Qatar Genome Program), Vietnamese reference genome, Ancient Sardinia reference genome, Danish reference genome (GENOME DK), or Siberian reference genome, but is not necessarily limited thereto.

In an embodiment of the present invention, the Hardy-Weinberg equilibrium (HWE) is a dataset extracted from genome-wide association studies (GWAS) deposited in the NCBI dbGaP (database of Genotypes and Phenotypes).

In an embodiment of the present invention, the allele frequency may be a Korean allele frequency. In one specific example of the present invention, the Korean allele frequency is extracted from the KoVariome database.

In an embodiment of the present invention, the genomic regions is an exon or coding sequence.

In an embodiment of the present invention, the SNPs not in linkage disequilibrium are extracted from HaploReg v 4.1 database. In one specific embodiment of the present invention, the SNPs not in linkage disequilibrium are SNPs having an r² value of less than 0.2, extracted from the HaploReg v 4.1 database.

In an embodiment of the present invention, the repeated region is a region disclosed in www.repeatmasker.org/species/hg.html.

Since the composition for kinship identification in Korean according to another aspect of the present invention is derived as a result of the above-described method of discovering SNP markers for kinship identification, any description that may be redundant will be omitted for clarity of this specification.

### Advantageous Effects

Characteristics and advantages of the present invention are summarized as follows:
(a) the present invention provides a composition for kinship identification in Korean, comprising an agent for amplifying or detecting a single nucleotide polymorphism (SNP) located at position 101 in at least one sequence selected from the group consisting of nucleotide sequences set forth in SEQ ID NO: 1 to SEQ ID NO: 918, SEQ ID NO: 919 to SEQ ID NO: 1400, or SEQ ID NO: 1 to SEQ ID NO: 1400;
(b) the present invention provides a kit for kinship identification in Korean, comprising the above-described composition for kinship identification in Korean;
(c) the present invention provides a method of kinship identification in Korean;
(d) the present invention provides a method of discovering an SNP marker for kinship identification; and
(e) the use of the composition for kinship identification in Korean of the present invention allows first-degree relatives to be clearly distinguished from those who are not first-degree relatives in a Korean population by using the minimum number of forensic SNP markers even when no DNA of immediate family member is available.

### DESCRIPTION OF DRAWINGS

FIG. 1 shows a flowchart for discovering 918 SNP markers for kinship identification.

FIG. 2 shows a flowchart for discovering 482 SNP markers for kinship identification.

FIG. 3 shows a schematic diagram of chromosomes with the positions of 918 and 482 SNP markers according to the present invention.

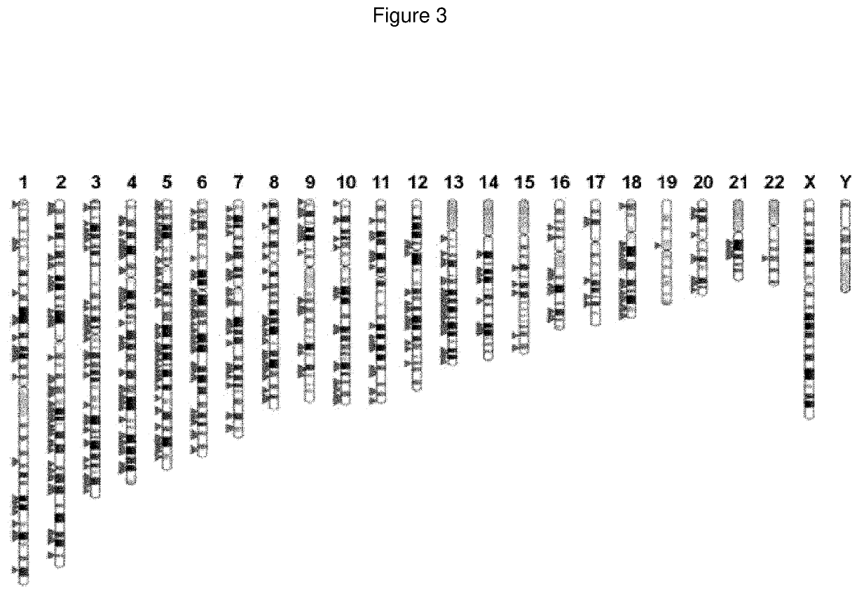

FIG. 4 shows a diagram of a WGS analysis pipeline.

FIG. 5 shows a comparison matrix of coefficients of relatedness as a result of IBS testing of 40 individuals using 918 SNP markers.

FIG. 6 shows a per-group comparison graph of coefficients of relatedness as a result of IBS testing of 40 individuals using 918 SNP markers.

FIG. 7 shows a comparison matrix of coefficients of relatedness as a result of IBS testing of 50 individuals using 918 SNP markers.

FIG. 8 shows a per-group comparison graph of coefficients of relatedness as a result of IBS testing of 50 individuals using 918 SNP markers.

FIG. 9 shows a comparison matrix of coefficients of relatedness as a result of IBS testing of 40 individuals using 482 SNP markers.

FIG. 10 shows a per-group comparison graph of coefficients of relatedness as a result of IBS testing of 40 individuals using 482 SNP markers.

FIG. 11 shows a comparison matrix of coefficients of relatedness as a result of IBS testing of 50 individuals using 482 SNP markers.

FIG. 12 shows a per-group comparison graph of relatedness as a result of IBS testing of 50 individuals using 482 SNP markers.

### MODE FOR INVENTION

Hereinbelow, the present invention will be described in greater detail in conjunction with examples. The following examples are provided to describe the present invention in further details, and it will become apparent to those skilled in the art that the scope of the present invention as suggested in the appended claims is not limited by the following examples.

### EXAMPLES

Throughout this specification, the sign “%” used to express the concentration of a substance, unless otherwise specified, refers to (weight/weight) % for solid/solid, (weight/volume) % for solid/liquid, and (volume/volume) % for liquid/liquid.

### Example 1. Outline of the Kinship Identification Method of the Present Invention

Currently, kinship testing based on markers such as forensic STRs is widely used. Although about 10⁵ to 10⁶ SNPs can be used to estimate relatedness of higher degrees, genome-wide genotyping and SNP analysis may be impractical for forensic use. Therefore, to provide it is worthwhile to explore SNP markers capable of estimating relatedness in a small quantity. In the present invention, for accurate analysis of kinship, SNP markers capable of estimating relatedness based on WGS were extracted and analyzed.

In order to discover the minimum number of SNPs required for genetic association identification to later create SNP panels required for kinship identification, 918 SNP markers and 482 SNP markers for kinship identification in a Korean population were discovered from about 84 million SNPs of 88 unrelated Korean individuals disclosed in Korean National Standard Reference Variome (KoVariome) database.

In order to identify kinship in Korean by using the 918 SNP markers and the 482 SNP markers discovered, the present inventors collected oral swaps or saliva from a Korean population of 90 individuals who are in first-degree, second-degree, and third-degree relationships. From the samples, DNAs were extracted and sequenced. Using a sequencing device, Nova-Seq (Illumina), whole genome sequencing (WGS) data of 40 individuals were produced. By providing WGS data of 40 individuals generated by Nova-Seq and samples of 50 individuals to Clinomics Inc., WGS data of 48 individuals using DNBSEQ-T7 (MGI) and WGS data of 2 individuals using Nova-Seq were obtained.

From the above described i) WGS data of 40 individuals produced from Nova-Seq, and ii) WGS data of 48 individuals produced from DNBSEQ-T7 and WGS data of 2 individuals produced from Nova-Seq, average IBS (identity by state) scores were derived through pairwise comparison between two individuals, and then divided into first degree relatives, second degree relatives, third degree relatives, and the unrelated (no kinship or unrelated) groups, and then compared for average IBS values to thereby perform an estimation analysis of kinship in Korean using the SNP markers of the present invention.

### Example 2. Method of Discovering SNP Markers for Kinship Identification in Korean

As described above, the present inventors have discovered SNP markers for kinship identification in Korean, from about 84 million SNPs of 88 unrelated Korean individuals, that is, individuals who are not in any kinship relationship, disclosed in Korean National Standard Reference Variome (KoVariome) database (Kim J et al. Sci Rep. 2018).

### 2-1: Discovery of 918 SNP Markers

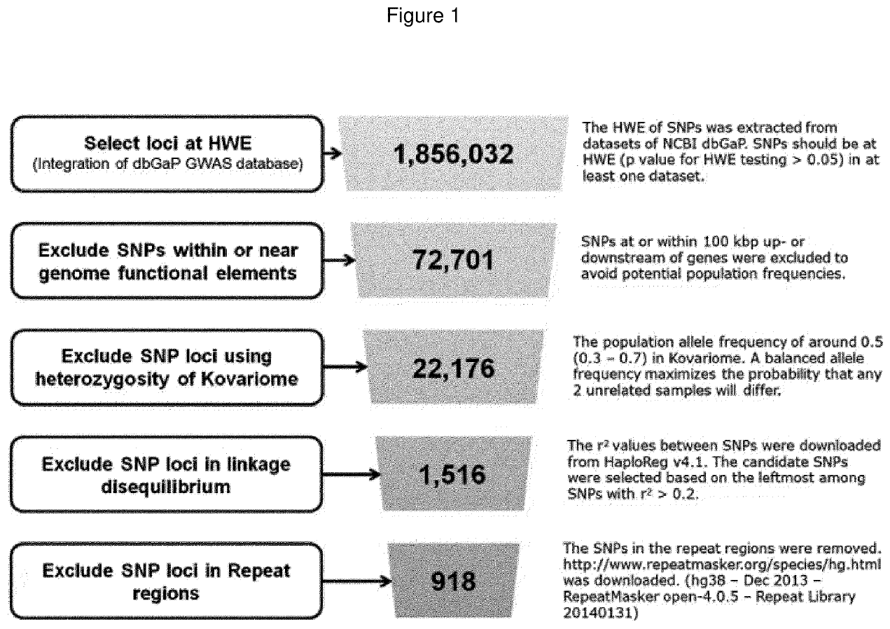

The process of selecting candidate SNP markers for kinship identification was performed in the sequence shown in the schematic diagram of FIG. 1.

### 2-1-1: Select Loci at Hardy-Weinberg Equilibrium

First, the Hardy-Weinberg equilibrium (HWE) of SNPs was extracted from genome-wide association studies (GWAS) deposited in the NCBI (National Center for Biotechnology Information) dbGaP (database of Genotype and Phenotypes). SNPs having p value of 0.05 or more at HWE test in at least one dataset were extracted. As a result, 1,856,032 SNPs were extracted.

2-1-2: Exclude SNPs within or Near Genomic Regions

SNPs at or within 100 kbp up- or downstream of genomic regions were excluded to avoid potential influence of selection pressure on SNP population frequencies. In genetic research on diseases, since phenotypes are important, exon research should be conducted. However, in forensic criminal investigations, investigations in genetic characteristics, particularly in case of genes coding for certain proteins, may lead to violation of human rights and thus are excluded from investigation or analysis applications. As a result, 72,701 SNPs were extracted.

### 2-1-3: Exclude SNP Loci Using Heterozygosity of Kovariome

In addition, SNPs located near the population allele frequency of around 0.5 (0.3 to 0.7) in Kovariome, which is Korea National Standard Reference Variome database described above, were extracted. A balanced allele frequency maximizes the probability that SNPs in any 2 unrelated samples will differ. As a result, 22,176 SNPs were extracted.

### 2-1-4: Exclude SNP Loci in Linkage Disequilibrium

To exclude SNPs in linkage disequilibrium (LD), SNPs with r² of 0.2 or more were excluded from SNPs extracted using HaploReg v 4.1 database (see Ward & Kellis, 2016). The candidate SNPs were selected based on the leftmost among SNPs with r² of 0.2 or more. As a result, 1,516 SNPs were extracted.

### 2-1-5: Exclude SNP Loci in Repeated Regions

Finally, SNPs in the known repeated regions were removed. The SNPs in the repeated regions were downloaded from www.repeatmasker.org/species/hg.html (hg38—December 2013—RepeatMasker open—4.0.5—Repeat Library 20140131). Finally, a panel of 918 SNPs was discovered.

The 918 SNPs selected for kinship identification in Korean are shown in Table 1 below, and sequences each consisting of 201 nucleotides including each SNP at position 101 are shown in SEQ ID NO: 1 to SEQ ID NO: 918. The position of an SNP on the chromosome is represented based on human reference genome GRCh38 (Genome Reference Consortium Human Build 38; hg38), and each SNP may have a reference allele (ref. allele) or an alternative allele (alt. allele) positioned at position 101 [n]. In addition, reference SNP cluster ID numbers (rs #) of dbSNP (Single Nucleotide Polymorphism database), which is an open database provided by NCBI (National Center for Biotechnology Information) were cited together.

TABLE 1

| SEQ ID | Chromosome | Position | Ref. | Alt. | |
|---|---|---|---|---|---|
| NO: | # | (GRCh38) | Allele | Allele | dbSNP rs# |
| 1 | 1 | 4281783 | C | T | rs1390136 |
| 2 | 1 | 4307966 | A | G | rs1817913 |
| 3 | 1 | 4308891 | C | T | rs351617 |
| 4 | 1 | 5193376 | G | A | rs12041491 |
| 5 | 1 | 29598423 | T | C | rs915409 |
| 6 | 1 | 30525732 | A | G | rs10915147 |
| 7 | 1 | 34379235 | G | A | rs705197 |
| 8 | 1 | 34478162 | G | A | rs10914958 |
| 9 | 1 | 60258050 | A | C | rs1156177 |
| 10 | 1 | 60410474 | T | C | rs7529292 |
| 11 | 1 | 79651949 | A | G | rs4650425 |
| 12 | 1 | 79767596 | C | T | rs3927739 |
| 13 | 1 | 79771567 | T | G | rs12119501 |
| 14 | 1 | 79783314 | A | C | rs12030273 |
| 15 | 1 | 80890585 | A | G | rs12145866 |
| 16 | 1 | 82095875 | T | G | rs962249 |
| 17 | 1 | 82100386 | A | T | rs11163422 |
| 18 | 1 | 83274975 | C | T | rs2538251 |
| 19 | 1 | 83277034 | C | G | rs17101590 |
| 20 | 1 | 83288496 | A | G | rs12120283 |
| 21 | 1 | 91128933 | T | A | rs240510 |
| 22 | 1 | 91139976 | G | A | rs653759 |
| 23 | 1 | 91152839 | A | G | rs347026 |
| 24 | 1 | 96123116 | T | G | rs2154010 |
| 25 | 1 | 96931655 | C | T | rs17115963 |
| 26 | 1 | 98376273 | T | C | rs7525553 |
| 27 | 1 | 98462715 | A | G | rs4950129 |
| 28 | 1 | 104328360 | T | G | rs11809638 |
| 29 | 1 | 104444439 | G | A | rs7520815 |
| 30 | 1 | 104651212 | A | T | rs11184241 |
| 31 | 1 | 106257917 | C | T | rs4301658 |
| 32 | 1 | 118365750 | G | T | rs17038218 |
| 33 | 1 | 165081809 | A | G | rs1494413 |
| 34 | 1 | 187896223 | C | T | rs10912208 |
| 35 | 1 | 187903620 | T | G | rs11591211 |
| 36 | 1 | 188351784 | A | T | rs4282763 |
| 37 | 1 | 188393167 | A | G | rs12039277 |
| 38 | 1 | 189253831 | C | T | rs6700979 |
| 39 | 1 | 191333287 | A | G | rs1246707 |
| 40 | 1 | 195186500 | A | T | rs681013 |
| 41 | 1 | 195189835 | A | T | rs4111374 |
| 42 | 1 | 195600484 | C | T | rs10494727 |
| 43 | 1 | 195863867 | A | G | rs7542834 |
| 44 | 1 | 195885507 | G | A | rs921516 |
| 45 | 1 | 199544437 | G | A | rs12045830 |
| 46 | 1 | 199613744 | C | A | rs9628679 |
| 47 | 1 | 208378428 | G | A | rs11119057 |
| 48 | 1 | 208463524 | T | C | rs993324 |
| 49 | 1 | 208855095 | T | C | rs12043779 |
| 50 | 1 | 220997968 | G | A | rs2484696 |
| 51 | 10 | 1847907 | C | A | rs17157139 |
| 52 | 10 | 9140785 | G | A | rs12243321 |
| 53 | 10 | 9417332 | T | C | rs12783041 |
| 54 | 10 | 9559927 | T | A | rs7920503 |
| 55 | 10 | 10563087 | C | T | rs2147291 |
| 56 | 10 | 10596873 | C | T | rs972186 |
| 57 | 10 | 10602864 | A | G | rs7089232 |
| 58 | 10 | 10610702 | G | A | rs4749972 |
| 59 | 10 | 29041960 | C | G | rs1335701 |
| 60 | 10 | 29081550 | T | A | rs9664566 |
| 61 | 10 | 29160200 | A | C | rs511991 |
| 62 | 10 | 53190596 | A | G | rs2384170 |
| 63 | 10 | 55847166 | A | G | rs1338788 |
| 64 | 10 | 56745375 | A | G | rs7924028 |
| 65 | 10 | 56867670 | A | G | rs11005565 |
| 66 | 10 | 57408260 | A | G | rs11005792 |
| 67 | 10 | 81367131 | C | A | rs649207 |
| 68 | 10 | 81404894 | C | A | rs7073999 |
| 69 | 10 | 81406583 | A | G | rs2183174 |
| 70 | 10 | 81545981 | A | G | rs17638653 |
| 71 | 10 | 83169451 | G | T | rs11197616 |
| 72 | 10 | 83560360 | C | T | rs11199196 |
| 73 | 10 | 84662987 | A | G | rs11599612 |
| 74 | 10 | 84705330 | C | G | rs2505778 |
| 75 | 10 | 84828122 | G | A | rs7901050 |
| 76 | 10 | 105411136 | G | A | rs12356045 |
| 77 | 10 | 106306479 | T | A | rs821676 |
| 78 | 10 | 120209055 | G | C | rs914483 |
| 79 | 10 | 120248718 | C | T | rs12243526 |
| 80 | 10 | 121292896 | A | C | rs12220387 |
| 81 | 10 | 124231226 | G | A | rs28564851 |
| 82 | 10 | 128420777 | C | T | rs1416722 |
| 83 | 10 | 128487399 | C | T | rs1984218 |
| 84 | 10 | 128520194 | C | T | rs11016297 |
| 85 | 10 | 128523624 | C | T | rs11016304 |
| 86 | 10 | 128549487 | G | C | rs10741157 |
| 87 | 10 | 128549508 | T | C | rs4750710 |
| 88 | 10 | 128554967 | A | G | rs7898393 |
| 89 | 10 | 128685050 | A | G | rs12253697 |
| 90 | 10 | 128712175 | A | G | rs7893266 |
| 91 | 10 | 128745696 | A | G | rs7910094 |
| 92 | 10 | 128749437 | C | G | rs11016458 |
| 93 | 10 | 128779321 | C | T | rs10829492 |
| 94 | 10 | 129123978 | A | G | rs10741181 |
| 95 | 10 | 129133073 | C | T | rs2123420 |
| 96 | 10 | 129135480 | T | C | rs914570 |
| 97 | 10 | 129149574 | A | G | rs10128422 |
| 98 | 10 | 130792707 | A | G | rs7917491 |
| 99 | 10 | 130804395 | C | T | rs12570660 |
| 100 | 10 | 131519607 | G | A | rs10765078 |
| 101 | 10 | 131562136 | T | C | rs10765092 |
| 102 | 10 | 131660805 | A | C | rs4545466 |
| 103 | 11 | 15354206 | C | T | rs11023541 |
| 104 | 11 | 15354744 | C | G | rs7122143 |
| 105 | 11 | 24088370 | G | A | rs1317014 |
| 106 | 11 | 24111009 | T | C | rs7121343 |
| 107 | 11 | 36852864 | C | G | rs10501163 |
| 108 | 11 | 37095265 | G | A | rs746517 |
| 109 | 11 | 37124101 | T | C | rs2704917 |
| 110 | 11 | 37228417 | G | A | rs4756376 |
| 111 | 11 | 39725355 | T | C | rs11035428 |
| 112 | 11 | 39975331 | G | A | rs6485169 |
| 113 | 11 | 79716863 | G | A | rs717100 |
| 114 | 11 | 79772010 | T | G | rs2663205 |
| 115 | 11 | 80091064 | C | A | rs570008 |
| 116 | 11 | 95335790 | C | T | rs7125428 |
| 117 | 11 | 97429250 | T | C | rs4755065 |
| 118 | 11 | 97761882 | T | G | rs1828511 |
| 119 | 11 | 98792099 | A | G | rs2169376 |
| 120 | 11 | 113581871 | T | C | rs12365214 |
| 121 | 11 | 114998713 | T | C | rs4489780 |
| 122 | 11 | 116164654 | C | T | rs2844282 |
| 123 | 11 | 116165087 | A | G | rs1783221 |
| 124 | 11 | 116179554 | G | A | rs1783235 |
| 125 | 11 | 116248195 | T | G | rs1009746 |
| 126 | 11 | 116344392 | G | T | rs11601506 |
| 127 | 11 | 116394352 | A | G | rs537538 |
| 128 | 11 | 127812099 | T | C | rs1157862 |
| 129 | 11 | 128346944 | C | T | rs4937324 |
| 130 | 11 | 133651877 | A | G | rs10791302 |
| 131 | 11 | 133661760 | C | T | rs11223548 |
| 132 | 12 | 28684011 | T | C | rs7312487 |
| 133 | 12 | 29968780 | T | C | rs16935137 |
| 134 | 12 | 29972734 | C | G | rs1909174 |
| 135 | 12 | 33096898 | A | G | rs1392331 |
| 136 | 12 | 33234373 | G | A | rs7312915 |
| 137 | 12 | 33252392 | T | C | rs7299903 |
| 138 | 12 | 61052965 | T | C | rs1395540 |
| 139 | 12 | 61054396 | G | C | rs7974374 |
| 140 | 12 | 61338759 | A | G | rs2176199 |
| 141 | 12 | 72881620 | G | A | rs6582124 |
| 142 | 12 | 83277649 | T | C | rs7485422 |
| 143 | 12 | 83464158 | C | T | rs11115877 |
| 144 | 12 | 87368810 | G | A | rs7968713 |
| 145 | 12 | 87537289 | C | T | rs11104542 |
| 146 | 12 | 90407150 | G | A | rs7969733 |
| 147 | 12 | 102625209 | T | C | rs7298152 |
| 148 | 12 | 108015068 | C | G | rs1896061 |
| 149 | 12 | 115466253 | T | C | rs721219 |
| 150 | 13 | 22403650 | T | C | rs9510177 |
| 151 | 13 | 22458981 | A | G | rs292476 |
| 152 | 13 | 22460138 | A | T | rs2048148 |
| 153 | 13 | 34804128 | C | T | rs9572437 |
| 154 | 13 | 37323438 | G | A | rs2209245 |
| 155 | 13 | 46999481 | A | G | rs12583882 |
| 156 | 13 | 48872502 | T | C | rs9596029 |
| 157 | 13 | 55282927 | C | T | rs9537053 |
| 158 | 13 | 55704977 | A | G | rs8001026 |
| 159 | 13 | 55748392 | G | T | rs1413111 |
| 160 | 13 | 55755583 | G | A | rs9597255 |
| 161 | 13 | 57831590 | G | C | rs9563541 |
| 162 | 13 | 58631984 | A | G | rs9563622 |
| 163 | 13 | 58880034 | G | A | rs17055603 |
| 164 | 13 | 59364835 | A | G | rs1409254 |
| 165 | 13 | 61533404 | A | G | rs9563933 |
| 166 | 13 | 64197296 | A | C | rs4144113 |
| 167 | 13 | 65074363 | G | A | rs4306391 |
| 168 | 13 | 65411117 | G | C | rs9571399 |
| 169 | 13 | 66074646 | C | T | rs9540627 |
| 170 | 13 | 68145856 | G | T | rs11148761 |
| 171 | 13 | 68445189 | A | T | rs9564463 |
| 172 | 13 | 70298257 | T | C | rs10161640 |
| 173 | 13 | 70698715 | C | T | rs17087430 |
| 174 | 13 | 72328053 | T | C | rs490599 |
| 175 | 13 | 74682207 | C | T | rs9543745 |
| 176 | 13 | 74703629 | C | T | rs1327740 |
| 177 | 13 | 74707684 | A | G | rs2104615 |
| 178 | 13 | 74999239 | G | A | rs974510 |
| 179 | 13 | 76281273 | C | T | rs2174452 |
| 180 | 13 | 80235685 | A | C | rs17072502 |
| 181 | 13 | 80472630 | T | C | rs944751 |
| 182 | 13 | 81402764 | C | T | rs1334384 |
| 183 | 13 | 81791630 | T | C | rs12429667 |
| 184 | 13 | 82013133 | C | T | rs17174633 |
| 185 | 13 | 82378443 | A | T | rs9601891 |
| 186 | 13 | 83398350 | T | C | rs4074473 |
| 187 | 13 | 83766892 | A | G | rs1331944 |
| 188 | 13 | 86564329 | A | C | rs9284259 |
| 189 | 13 | 87992497 | G | T | rs11619659 |
| 190 | 13 | 88341210 | A | C | rs1993354 |
| 191 | 13 | 90657178 | T | C | rs12430684 |
| 192 | 13 | 103175477 | G | A | rs2765584 |
| 193 | 13 | 103179406 | T | C | rs9518946 |
| 194 | 13 | 103722188 | G | T | rs4459430 |
| 195 | 13 | 103730905 | G | A | rs1274749 |
| 196 | 13 | 103944701 | A | G | rs2259614 |
| 197 | 13 | 104007140 | C | T | rs1849445 |
| 198 | 13 | 104245414 | A | G | rs9586450 |
| 199 | 13 | 104335370 | A | G | rs1330527 |
| 200 | 13 | 104357291 | G | C | rs9519304 |
| 201 | 13 | 104358144 | A | G | rs9514252 |
| 202 | 13 | 104511073 | C | T | rs7981507 |
| 203 | 13 | 104598394 | G | T | rs2164080 |
| 204 | 13 | 104623579 | C | T | rs7337127 |
| 205 | 13 | 104630753 | A | C | rs496815 |
| 206 | 13 | 104675786 | G | A | rs773350 |
| 207 | 13 | 104680105 | G | T | rs773312 |
| 208 | 13 | 104916959 | G | A | rs7320561 |
| 209 | 13 | 104947926 | C | T | rs9555118 |
| 210 | 13 | 104986744 | T | C | rs9519543 |
| 211 | 13 | 104987191 | T | C | rs9634456 |
| 212 | 13 | 104987592 | T | A | rs9300980 |
| 213 | 13 | 104989927 | C | A | rs9300981 |
| 214 | 13 | 105211762 | A | G | rs3015350 |
| 215 | 13 | 105276911 | C | T | rs7989487 |
| 216 | 13 | 105285190 | T | C | rs9519641 |
| 217 | 13 | 105288707 | G | C | rs9301014 |
| 218 | 13 | 108059883 | C | T | rs1325389 |
| 219 | 13 | 111448590 | T | C | rs9560075 |
| 220 | 14 | 26334093 | C | A | rs1956509 |
| 221 | 14 | 27951885 | A | G | rs11624431 |
| 222 | 14 | 27997327 | A | G | rs1888385 |
| 223 | 14 | 27997820 | C | T | rs2251721 |
| 224 | 14 | 27997930 | A | G | rs2775279 |
| 225 | 14 | 27998525 | A | G | rs6575994 |
| 226 | 14 | 28018102 | T | C | rs1013379 |
| 227 | 14 | 38497401 | A | G | rs11628255 |
| 228 | 14 | 43762866 | T | C | rs1957280 |
| 229 | 14 | 48944485 | G | A | rs2352906 |
| 230 | 14 | 49189123 | G | A | rs8011197 |
| 231 | 14 | 62252583 | A | T | rs1399523 |
| 232 | 14 | 80077778 | A | G | rs1181353 |
| 233 | 14 | 82132173 | T | A | rs17116374 |
| 234 | 14 | 82531231 | T | C | rs12435137 |
| 235 | 14 | 83170173 | G | C | rs7152994 |
| 236 | 14 | 83481552 | A | G | rs4243668 |
| 237 | 14 | 83533399 | G | A | rs1241925 |
| 238 | 14 | 83628044 | T | C | rs7144922 |
| 239 | 14 | 84285197 | T | C | rs8019836 |
| 240 | 14 | 84844447 | G | A | rs1483065 |
| 241 | 14 | 85755382 | C | T | rs8009268 |
| 242 | 14 | 86629516 | G | A | rs4899905 |
| 243 | 15 | 37595807 | A | T | rs8023501 |
| 244 | 15 | 37671684 | T | C | rs16965448 |
| 245 | 15 | 37795999 | A | G | rs4924192 |
| 246 | 15 | 46206243 | G | A | rs1897841 |
| 247 | 15 | 53233218 | C | T | rs10163018 |
| 248 | 15 | 87152512 | A | G | rs11632375 |
| 249 | 15 | 87154857 | G | A | rs10152178 |
| 250 | 15 | 96948385 | T | C | rs1468626 |
| 251 | 15 | 96973890 | C | T | rs11630458 |
| 252 | 16 | 17667817 | T | C | rs8053673 |
| 253 | 16 | 25585119 | G | A | rs205163 |
| 254 | 16 | 26938339 | G | T | rs9806926 |
| 255 | 16 | 26946375 | T | C | rs11074811 |
| 256 | 16 | 26950631 | G | A | rs12447485 |
| 257 | 16 | 26963686 | T | G | rs4238945 |
| 258 | 16 | 52816487 | A | G | rs16951610 |
| 259 | 16 | 55149989 | C | T | rs1463632 |
| 260 | 16 | 60154373 | T | C | rs929854 |
| 261 | 16 | 60757487 | T | G | rs11076411 |
| 262 | 16 | 61270284 | G | T | rs16963190 |
| 263 | 16 | 62702897 | C | T | rs4131140 |
| 264 | 16 | 64047131 | T | C | rs16966908 |
| 265 | 16 | 64842774 | G | T | rs1005822 |
| 266 | 16 | 66040422 | G | T | rs11075616 |
| 267 | 16 | 66061838 | G | A | rs7184941 |
| 268 | 16 | 75962058 | A | G | rs1498059 |
| 269 | 16 | 82311978 | T | C | rs2906802 |
| 270 | 17 | 10990482 | A | C | rs7219444 |
| 271 | 17 | 10995136 | A | G | rs758112 |
| 272 | 17 | 14595973 | C | G | rs8074338 |
| 273 | 17 | 14604697 | T | A | rs11078260 |
| 274 | 17 | 52642873 | C | T | rs2452236 |
| 275 | 17 | 65356847 | G | A | rs6504297 |
| 276 | 17 | 70471938 | G | C | rs4080890 |
| 277 | 17 | 70883284 | A | G | rs17223678 |
| 278 | 17 | 71399303 | G | A | rs917345 |
| 279 | 17 | 71399807 | C | T | rs8071270 |
| 280 | 18 | 4564126 | A | G | rs8095810 |
| 281 | 18 | 4615729 | C | T | rs4372777 |
| 282 | 18 | 25458254 | T | G | rs4800216 |
| 283 | 18 | 25541579 | T | G | rs1627290 |
| 284 | 18 | 28283384 | C | T | rs1548755 |
| 285 | 18 | 29775114 | T | A | rs9635913 |
| 286 | 18 | 29823325 | A | G | rs16947191 |
| 287 | 18 | 29858338 | G | A | rs12960708 |
| 288 | 18 | 29923528 | T | C | rs12607637 |
| 289 | 18 | 30517355 | G | T | rs41379044 |
| 290 | 18 | 34330004 | A | G | rs12968586 |
| 291 | 18 | 38092452 | C | A | rs9966896 |
| 292 | 18 | 38127720 | C | T | rs4800001 |
| 293 | 18 | 38380962 | C | T | rs958801 |
| 294 | 18 | 38387518 | T | C | rs1511939 |
| 295 | 18 | 38464762 | C | T | rs991045 |
| 296 | 18 | 38465748 | C | T | rs10502693 |
| 297 | 18 | 38474197 | C | T | rs5000343 |
| 298 | 18 | 38474263 | C | T | rs2217109 |
| 299 | 18 | 38482221 | T | A | rs9959302 |
| 300 | 18 | 38483159 | C | T | rs12962345 |
| 301 | 18 | 38795294 | G | T | rs9946289 |
| 302 | 18 | 40350048 | T | A | rs16973135 |
| 303 | 18 | 40375825 | A | C | rs2862365 |
| 304 | 18 | 40437661 | C | A | rs16973361 |
| 305 | 18 | 40907143 | A | G | rs974309 |
| 306 | 18 | 40944295 | C | A | rs1869282 |
| 307 | 18 | 43386920 | C | T | rs684289 |
| 308 | 18 | 51950685 | A | G | rs2953258 |
| 309 | 18 | 53731968 | T | C | rs3906453 |
| 310 | 18 | 56327706 | C | T | rs12150697 |
| 311 | 18 | 56382009 | T | C | rs216568 |
| 312 | 18 | 61155719 | C | G | rs10513926 |
| 313 | 18 | 64524418 | T | C | rs8092218 |
| 314 | 18 | 64540623 | A | T | rs4531842 |
| 315 | 18 | 65288133 | C | T | rs1505996 |
| 316 | 18 | 67255462 | A | G | rs11875127 |
| 317 | 18 | 70783769 | A | G | rs17083161 |
| 318 | 18 | 70826008 | A | T | rs12606536 |
| 319 | 18 | 71340217 | A | G | rs1244840 |
| 320 | 18 | 71373343 | T | C | rs334428 |
| 321 | 18 | 72206367 | A | C | rs10871696 |
| 322 | 18 | 72240489 | G | T | rs7232482 |
| 323 | 18 | 72289278 | A | T | rs4530251 |
| 324 | 18 | 72291789 | C | G | rs4891990 |
| 325 | 18 | 72301864 | C | G | rs7230444 |
| 326 | 18 | 72351642 | C | A | rs9949765 |
| 327 | 18 | 72431957 | T | C | rs12456190 |
| 328 | 18 | 72982202 | A | G | rs9951195 |
| 329 | 18 | 73037239 | C | T | rs8093852 |
| 330 | 18 | 73565234 | C | T | rs4892113 |
| 331 | 18 | 73580087 | T | C | rs9967039 |
| 332 | 18 | 77751986 | A | G | rs8090827 |
| 333 | 18 | 77763393 | C | T | rs2727057 |
| 334 | 18 | 78242654 | A | C | rs4458091 |
| 335 | 18 | 78253589 | C | T | rs7239178 |
| 336 | 18 | 78306845 | T | C | rs612097 |
| 337 | 18 | 78650533 | A | G | rs2016252 |
| 338 | 18 | 78671563 | G | A | rs4798975 |
| 339 | 18 | 78675166 | G | A | rs12605652 |
| 340 | 2 | 2433302 | A | G | rs7577250 |
| 341 | 2 | 2522406 | G | A | rs10865530 |
| 342 | 2 | 2529716 | C | G | rs11676482 |
| 343 | 2 | 2536520 | A | C | rs2037148 |
| 344 | 2 | 4242754 | A | G | rs17018897 |
| 345 | 2 | 4246747 | T | G | rs1105752 |
| 346 | 2 | 4281788 | G | A | rs6760082 |
| 347 | 2 | 4359522 | C | A | rs2314218 |
| 348 | 2 | 5414407 | G | A | rs12466470 |
| 349 | 2 | 6106273 | G | A | rs6719357 |
| 350 | 2 | 22649032 | G | A | rs1484679 |
| 351 | 2 | 35826675 | C | A | rs4109145 |
| 352 | 2 | 36140420 | A | G | rs10174078 |
| 353 | 2 | 40868073 | G | A | rs1379026 |
| 354 | 2 | 41259375 | A | G | rs1991080 |
| 355 | 2 | 41420114 | A | G | rs17027678 |
| 356 | 2 | 41569117 | A | G | rs1975062 |
| 357 | 2 | 41601181 | G | A | rs12989506 |
| 358 | 2 | 53132207 | C | A | rs1900247 |
| 359 | 2 | 53266121 | G | C | rs195611 |
| 360 | 2 | 60213064 | G | C | rs11125832 |
| 361 | 2 | 80980289 | G | A | rs10520291 |
| 362 | 2 | 81766792 | A | G | rs1221175 |
| 363 | 2 | 82114606 | C | T | rs1402774 |
| 364 | 2 | 83323948 | G | C | rs6745924 |
| 365 | 2 | 104271947 | A | C | rs12992682 |
| 366 | 2 | 116040546 | G | A | rs1842420 |
| 367 | 2 | 117641566 | A | C | rs2048819 |
| 368 | 2 | 118461803 | G | T | rs7566562 |
| 369 | 2 | 118535500 | C | T | rs11674141 |
| 370 | 2 | 118641592 | C | G | rs1349459 |
| 371 | 2 | 123177446 | G | A | rs6734319 |
| 372 | 2 | 128684458 | T | C | rs28608534 |
| 373 | 2 | 128718773 | C | T | rs6431023 |
| 374 | 2 | 128745702 | T | G | rs10168422 |
| 375 | 2 | 128783968 | C | T | rs12473285 |
| 376 | 2 | 133698922 | G | A | rs6430444 |
| 377 | 2 | 133825743 | T | C | rs6430460 |
| 378 | 2 | 133866705 | T | C | rs4603824 |
| 379 | 2 | 133900169 | C | A | rs1446732 |
| 380 | 2 | 136648001 | C | T | rs1823084 |
| 381 | 2 | 137816741 | A | G | rs7581781 |
| 382 | 2 | 139076454 | A | T | rs10445695 |
| 383 | 2 | 142244191 | T | C | rs12463449 |
| 384 | 2 | 142660801 | C | A | rs2126000 |
| 385 | 2 | 146043609 | A | G | rs10803514 |
| 386 | 2 | 147032717 | A | G | rs16826756 |
| 387 | 2 | 147254218 | T | G | rs2890872 |
| 388 | 2 | 147600976 | C | T | rs12612855 |
| 389 | 2 | 150752251 | A | G | rs10497071 |
| 390 | 2 | 150876465 | A | G | rs12618428 |
| 391 | 2 | 152992091 | T | C | rs16831993 |
| 392 | 2 | 155831263 | T | C | rs1829525 |
| 393 | 2 | 155909118 | T | C | rs16839725 |
| 394 | 2 | 162971605 | T | C | rs1345959 |
| 395 | 2 | 162974977 | G | T | rs7599823 |
| 396 | 2 | 163441348 | C | A | rs7574166 |
| 397 | 2 | 166720864 | G | A | rs11887539 |
| 398 | 2 | 175564852 | A | G | rs9287985 |
| 399 | 2 | 180107615 | A | G | rs2056785 |
| 400 | 2 | 180132415 | G | A | rs12464448 |
| 401 | 2 | 180133957 | T | C | rs7606018 |
| 402 | 2 | 183714705 | T | C | rs13422535 |
| 403 | 2 | 184485875 | G | A | rs1992514 |
| 404 | 2 | 185061558 | G | C | rs17584976 |
| 405 | 2 | 191526144 | G | A | rs11889710 |
| 406 | 2 | 192322055 | C | T | rs4853669 |
| 407 | 2 | 192400180 | T | C | rs33957851 |
| 408 | 2 | 192468221 | A | G | rs1385350 |
| 409 | 2 | 192517040 | A | G | rs11886140 |
| 410 | 2 | 192877352 | T | C | rs2218495 |
| 411 | 2 | 193379097 | G | T | rs4420715 |
| 412 | 2 | 195131352 | T | C | rs13383422 |
| 413 | 2 | 195170744 | G | A | rs7583521 |
| 414 | 2 | 220956193 | A | G | rs11682003 |
| 415 | 2 | 221060913 | G | A | rs4672993 |
| 416 | 2 | 221818216 | A | C | rs2551201 |
| 417 | 2 | 225188804 | G | A | rs12694656 |
| 418 | 2 | 225217439 | A | G | rs6736537 |
| 419 | 2 | 225250729 | G | A | rs7571208 |
| 420 | 2 | 226546643 | A | C | rs962171 |
| 421 | 2 | 235303360 | A | G | rs1367876 |
| 422 | 2 | 235336266 | C | G | rs6414039 |
| 423 | 2 | 235392731 | G | A | rs10201495 |
| 424 | 20 | 6956198 | C | T | rs6077109 |
| 425 | 20 | 7472688 | T | C | rs4813827 |
| 426 | 20 | 12030641 | T | G | rs13037930 |
| 427 | 20 | 12030723 | A | G | rs6109233 |
| 428 | 20 | 12030940 | A | G | rs2327592 |
| 429 | 20 | 12044061 | A | G | rs1997810 |
| 430 | 20 | 12488857 | C | A | rs8182799 |
| 431 | 20 | 12675520 | T | C | rs6134626 |
| 432 | 20 | 12744370 | C | G | rs6041591 |
| 433 | 20 | 24783547 | C | T | rs226665 |
| 434 | 20 | 39449471 | C | T | rs16988070 |
| 435 | 20 | 40241479 | T | C | rs1980593 |
| 436 | 20 | 40242000 | G | C | rs2870432 |
| 437 | 20 | 40284166 | G | A | rs16988918 |
| 438 | 20 | 40354288 | T | C | rs10485671 |
| 439 | 20 | 41821595 | A | T | rs6093527 |
| 440 | 20 | 51252774 | A | G | rs1007126 |
| 441 | 20 | 54960007 | T | G | rs928165 |
| 442 | 20 | 55178061 | T | C | rs11086494 |
| 443 | 20 | 55266878 | C | T | rs6069223 |
| 444 | 20 | 56887842 | G | A | rs4810051 |
| 445 | 21 | 16974047 | G | C | rs8134480 |
| 446 | 21 | 17089525 | C | T | rs2223079 |
| 447 | 21 | 20005235 | T | C | rs2825938 |
| 448 | 21 | 22699093 | T | G | rs1513732 |
| 449 | 21 | 23592329 | A | T | rs975592 |
| 450 | 21 | 24647980 | G | T | rs2829220 |
| 451 | 21 | 24662659 | G | C | rs2829240 |
| 452 | 21 | 24737242 | G | A | rs2829310 |
| 453 | 21 | 27247990 | C | G | rs2830800 |
| 454 | 21 | 27251036 | A | G | rs2830802 |
| 455 | 22 | 34348079 | T | C | rs204276 |
| 456 | 3 | 5556979 | T | C | rs1430005 |
| 457 | 3 | 5584074 | G | A | rs6765805 |
| 458 | 3 | 5678174 | G | A | rs1545506 |
| 459 | 3 | 5760199 | G | A | rs1836577 |
| 460 | 3 | 5858280 | C | T | rs722615 |
| 461 | 3 | 22484685 | A | T | rs6769828 |
| 462 | 3 | 22549447 | G | A | rs1463223 |
| 463 | 3 | 26506852 | C | G | rs9820211 |
| 464 | 3 | 26836475 | C | T | rs7623573 |
| 465 | 3 | 26837096 | A | C | rs11129244 |
| 466 | 3 | 26864096 | C | A | rs4443123 |
| 467 | 3 | 26974564 | T | C | rs12487330 |
| 468 | 3 | 26974902 | T | C | rs17019072 |
| 469 | 3 | 30110652 | C | T | rs1032921 |
| 470 | 3 | 30122198 | A | G | rs11715853 |
| 471 | 3 | 30996454 | A | C | rs4624599 |
| 472 | 3 | 35984485 | G | T | rs11129681 |
| 473 | 3 | 61450901 | C | T | rs9872718 |
| 474 | 3 | 74622030 | C | T | rs9840407 |
| 475 | 3 | 74787877 | C | G | rs1358345 |
| 476 | 3 | 77912353 | G | T | rs11927310 |
| 477 | 3 | 78402487 | A | G | rs9837024 |
| 478 | 3 | 80320585 | C | T | rs7617987 |
| 479 | 3 | 82565752 | C | A | rs13079811 |
| 480 | 3 | 95272414 | C | T | rs17384029 |
| 481 | 3 | 103369823 | G | A | rs4074892 |
| 482 | 3 | 103739235 | T | C | rs1379623 |
| 483 | 3 | 104172135 | G | A | rs1104964 |
| 484 | 3 | 104334184 | T | C | rs9809062 |
| 485 | 3 | 105163092 | A | G | rs6770943 |
| 486 | 3 | 105262407 | A | G | rs952365 |
| 487 | 3 | 105972718 | G | T | rs13062596 |
| 488 | 3 | 105976728 | T | C | rs2655033 |
| 489 | 3 | 110087876 | G | T | rs13060275 |
| 490 | 3 | 110389488 | C | T | rs2712965 |
| 491 | 3 | 117241720 | G | A | rs12487249 |
| 492 | 3 | 117296552 | T | C | rs11716697 |
| 493 | 3 | 117358372 | A | G | rs1398641 |
| 494 | 3 | 117437585 | A | C | rs3971401 |
| 495 | 3 | 117438449 | A | G | rs16826930 |
| 496 | 3 | 135739142 | T | C | rs1447600 |
| 497 | 3 | 144290458 | G | A | rs6770845 |
| 498 | 3 | 144558491 | T | C | rs2717389 |
| 499 | 3 | 144953465 | G | A | rs6779715 |
| 500 | 3 | 145220841 | G | A | rs1348658 |
| 501 | 3 | 146732481 | T | C | rs16858832 |
| 502 | 3 | 147699834 | C | A | rs12639104 |
| 503 | 3 | 147705722 | A | G | rs2319306 |
| 504 | 3 | 147713968 | T | C | rs9653947 |
| 505 | 3 | 152945842 | A | G | rs2043854 |
| 506 | 3 | 162705247 | C | T | rs7433221 |
| 507 | 3 | 162884798 | C | T | rs9823850 |
| 508 | 3 | 176215977 | A | G | rs9848455 |
| 509 | 3 | 182265049 | A | G | rs1542695 |
| 510 | 3 | 187552363 | G | A | rs7645149 |
| 51 | 3 | 189451891 | C | G | rs3896089 |
| 512 | 3 | 189498378 | T | C | rs2600846 |
| 513 | 3 | 191833690 | G | C | rs2138537 |
| 514 | 3 | 193032457 | A | G | rs9831317 |
| 515 | 3 | 193040906 | G | A | rs6789557 |
| 516 | 3 | 193041642 | A | G | rs9851295 |
| 517 | 4 | 10917246 | G | T | rs16875484 |
| 518 | 4 | 10959364 | G | T | rs11726335 |
| 519 | 4 | 10959669 | G | A | rs7682428 |
| 520 | 4 | 11048220 | G | A | rs1110365 |
| 521 | 4 | 11049711 | C | T | rs9291486 |
| 522 | 4 | 11189846 | G | T | rs6835872 |
| 523 | 4 | 11214614 | C | T | rs10009253 |
| 524 | 4 | 13174346 | A | T | rs16888303 |
| 525 | 4 | 18314369 | A | G | rs4140904 |
| 526 | 4 | 24008164 | T | G | rs624456 |
| 527 | 4 | 24136516 | C | T | rs7663527 |
| 528 | 4 | 24224295 | A | G | rs6820131 |
| 529 | 4 | 24282415 | T | C | rs7663025 |
| 530 | 4 | 24300108 | T | G | rs7664742 |
| 531 | 4 | 27693271 | T | G | rs1031311 |
| 532 | 4 | 28092597 | G | T | rs28539715 |
| 533 | 4 | 30132426 | A | G | rs2571494 |
| 534 | 4 | 30155271 | A | C | rs6824691 |
| 535 | 4 | 30166358 | C | T | rs11930738 |
| 536 | 4 | 30387552 | T | A | rs41445348 |
| 537 | 4 | 30452690 | A | T | rs6833068 |
| 538 | 4 | 32456905 | C | G | rs11935790 |
| 539 | 4 | 34840072 | T | A | rs1467099 |
| 540 | 4 | 35071923 | G | A | rs4859335 |
| 541 | 4 | 35134580 | G | A | rs7684845 |
| 542 | 4 | 35148038 | C | A | rs1023933 |
| 543 | 4 | 35151566 | G | A | rs6852760 |
| 544 | 4 | 35222908 | C | T | rs7691687 |
| 545 | 4 | 35332851 | T | C | rs1530242 |
| 546 | 4 | 35702700 | A | G | rs1035712 |
| 547 | 4 | 45154996 | T | C | rs7654230 |
| 548 | 4 | 45173787 | T | C | rs16858082 |
| 549 | 4 | 45793878 | G | A | rs1492839 |
| 550 | 4 | 45802030 | C | T | rs1389034 |
| 551 | 4 | 57970497 | T | C | rs269829 |
| 552 | 4 | 58289243 | C | T | rs17603347 |
| 553 | 4 | 59294093 | C | A | rs7440334 |
| 554 | 4 | 62963684 | T | G | rs2660605 |
| 555 | 4 | 62979085 | A | G | rs13123702 |
| 556 | 4 | 66533925 | C | T | rs1995913 |
| 557 | 4 | 66736552 | T | C | rs10005193 |
| 558 | 4 | 66760731 | G | C | rs1947248 |
| 559 | 4 | 67195915 | G | A | rs6815091 |
| 560 | 4 | 67204598 | C | A | rs6841066 |
| 561 | 4 | 71926132 | T | C | rs1520499 |
| 562 | 4 | 72698088 | T | C | rs9291177 |
| 563 | 4 | 85368167 | A | G | rs403870 |
| 564 | 4 | 91762989 | G | A | rs6811317 |
| 565 | 4 | 93939277 | C | T | rs2578139 |
| 566 | 4 | 93962222 | T | C | rs1898636 |
| 567 | 4 | 95976372 | C | T | rs6823094 |
| 568 | 4 | 95989085 | A | C | rs263075 |
| 569 | 4 | 95995607 | G | A | rs7689809 |
| 570 | 4 | 96040740 | C | T | rs1567957 |
| 571 | 4 | 114368048 | A | G | rs4989308 |
| 572 | 4 | 114392159 | G | T | rs1962495 |
| 573 | 4 | 115244948 | C | T | rs1112531 |
| 574 | 4 | 124823148 | G | A | rs1506556 |
| 575 | 4 | 125019845 | C | G | rs2390770 |
| 576 | 4 | 125019955 | T | C | rs1391392 |
| 577 | 4 | 126260508 | T | C | rs10857112 |
| 578 | 4 | 126320991 | C | A | rs2391038 |
| 579 | 4 | 130605780 | C | T | rs7664241 |
| 580 | 4 | 130700347 | C | G | rs41331449 |
| 581 | 4 | 133370732 | T | C | rs6818278 |
| 582 | 4 | 133763089 | G | T | rs17609744 |
| 583 | 4 | 141890740 | C | T | rs1500842 |
| 584 | 4 | 155008953 | G | A | rs6820565 |
| 585 | 4 | 156246739 | C | T | rs7700147 |
| 586 | 4 | 159894529 | C | T | rs7672637 |
| 587 | 4 | 159902822 | G | A | rs6849301 |
| 588 | 4 | 160020695 | C | G | rs2711281 |
| 589 | 4 | 160367917 | C | T | rs3846242 |
| 590 | 4 | 160693235 | T | C | rs17359785 |
| 591 | 4 | 160982953 | A | G | rs4260487 |
| 592 | 4 | 164488143 | T | C | rs6834683 |
| 593 | 4 | 166626278 | G | A | rs7673076 |
| 594 | 4 | 167510453 | T | C | rs10517942 |
| 595 | 4 | 176433799 | A | G | rs2122336 |
| 596 | 4 | 178510860 | A | G | rs2614604 |
| 597 | 4 | 178520937 | C | T | rs2715538 |
| 598 | 4 | 178787228 | C | G | rs2044699 |
| 599 | 4 | 178808271 | C | A | rs4862165 |
| 600 | 4 | 179202114 | G | A | rs10028580 |
| 601 | 4 | 179249214 | C | G | rs7435849 |
| 602 | 4 | 179626828 | C | T | rs17775338 |
| 603 | 4 | 179634825 | C | T | rs6834985 |
| 604 | 4 | 179752080 | T | C | rs2100685 |
| 605 | 4 | 180218268 | C | T | rs17068958 |
| 606 | 4 | 180281999 | G | A | rs17069057 |
| 607 | 4 | 180301728 | C | T | rs13109268 |
| 608 | 4 | 180367017 | T | C | rs2309249 |
| 609 | 4 | 180391684 | G | A | rs6811892 |
| 610 | 4 | 180392768 | C | T | rs11734423 |
| 611 | 4 | 180432579 | A | G | rs2309341 |
| 612 | 4 | 180444072 | C | T | rs2727430 |
| 613 | 4 | 180861463 | C | T | rs12644875 |
| 614 | 4 | 180862831 | T | C | rs17070257 |
| 615 | 4 | 181370290 | A | G | rs17246459 |
| 616 | 5 | 2370332 | G | C | rs11949075 |
| 617 | 5 | 2371828 | G | A | rs10036994 |
| 618 | 5 | 2378722 | T | C | rs465850 |
| 619 | 5 | 2398516 | A | C | rs650640 |
| 620 | 5 | 2403330 | G | A | rs10059366 |
| 621 | 5 | 2448046 | A | G | rs6555108 |
| 622 | 5 | 2462244 | C | A | rs12655007 |
| 623 | 5 | 2466470 | C | T | rs315918 |
| 624 | 5 | 2468261 | C | A | rs924134 |
| 625 | 5 | 2491471 | A | T | rs462437 |
| 626 | 5 | 2491562 | T | C | rs16869827 |
| 627 | 5 | 2493166 | A | C | rs2453814 |
| 628 | 5 | 3068876 | A | T | rs2447672 |
| 629 | 5 | 3756952 | T | C | rs13361372 |
| 630 | 5 | 3779477 | A | G | rs6882956 |
| 631 | 5 | 3799719 | G | A | rs4702464 |
| 632 | 5 | 3856335 | C | T | rs12153260 |
| 633 | 5 | 4286823 | G | C | rs9313074 |
| 634 | 5 | 5799874 | T | A | rs7703147 |
| 635 | 5 | 5828535 | T | C | rs188684 |
| 636 | 5 | 5887319 | G | T | rs1174922 |
| 637 | 5 | 5891765 | T | C | rs1174930 |
| 638 | 5 | 12017153 | A | G | rs4544819 |
| 639 | 5 | 12054151 | T | G | rs6862695 |
| 640 | 5 | 12060558 | T | C | rs10462685 |
| 641 | 5 | 13358322 | C | T | rs4398634 |
| 642 | 5 | 18337358 | T | G | rs10942113 |
| 643 | 5 | 19337298 | T | C | rs2063564 |
| 644 | 5 | 23155971 | G | T | rs310912 |
| 645 | 5 | 23790902 | C | T | rs12186540 |
| 646 | 5 | 25554641 | T | G | rs6885503 |
| 647 | 5 | 26125530 | A | G | rs1330660 |
| 648 | 5 | 26139251 | G | A | rs13154516 |
| 649 | 5 | 26192988 | A | G | rs1411586 |
| 650 | 5 | 26231825 | A | G | rs4604153 |
| 651 | 5 | 26238085 | C | T | rs9293229 |
| 652 | 5 | 27598430 | T | G | rs4235543 |
| 653 | 5 | 27810333 | T | A | rs6864726 |
| 654 | 5 | 27853422 | T | C | rs4128292 |
| 655 | 5 | 28428784 | A | G | rs4867546 |
| 656 | 5 | 30084498 | T | C | rs4867249 |
| 657 | 5 | 30843732 | A | G | rs2066930 |
| 658 | 5 | 30883945 | T | C | rs1921092 |
| 659 | 5 | 30919759 | C | T | rs13356028 |
| 660 | 5 | 34474631 | A | G | rs6451130 |
| 661 | 5 | 44185810 | G | A | rs4293927 |
| 662 | 5 | 50265025 | C | T | rs1328254 |
| 663 | 5 | 51769467 | A | G | rs4391124 |
| 664 | 5 | 52383332 | A | C | rs4865730 |
| 665 | 5 | 52404601 | A | C | rs12521835 |
| 666 | 5 | 58277369 | A | G | rs37525 |
| 667 | 5 | 62029131 | A | G | rs1833868 |
| 668 | 5 | 62879988 | A | G | rs1402912 |
| 669 | 5 | 62887158 | T | C | rs2222395 |
| 670 | 5 | 63126263 | C | T | rs10039523 |
| 671 | 5 | 63788359 | G | A | rs1478493 |
| 672 | 5 | 63818580 | A | G | rs1478490 |
| 673 | 5 | 68668261 | T | C | rs255257 |
| 674 | 5 | 71949983 | T | C | rs1217744 |
| 675 | 5 | 84682678 | G | A | rs6866089 |
| 676 | 5 | 84707871 | T | G | rs10076429 |
| 677 | 5 | 84722872 | C | T | rs4489064 |
| 678 | 5 | 84722873 | A | G | rs4256345 |
| 679 | 5 | 84823969 | G | T | rs7709606 |
| 680 | 5 | 85956586 | C | A | rs12652144 |
| 681 | 5 | 89788656 | T | C | rs819355 |
| 682 | 5 | 91945393 | G | T | rs825388 |
| 683 | 5 | 99701395 | G | T | rs7717666 |
| 684 | 5 | 100260665 | G | A | rs326100 |
| 685 | 5 | 101683566 | A | G | rs12654056 |
| 686 | 5 | 103682011 | T | C | rs1152176 |
| 687 | 5 | 103689757 | T | C | rs9632474 |
| 688 | 5 | 103705903 | C | T | rs3906355 |
| 689 | 5 | 103750446 | G | A | rs2161495 |
| 690 | 5 | 105528850 | A | G | rs4128686 |
| 691 | 5 | 106050008 | T | G | rs960931 |
| 692 | 5 | 106649102 | T | G | rs6876851 |
| 693 | 5 | 110030010 | A | G | rs2168308 |
| 694 | 5 | 110033459 | G | T | rs4957850 |
| 695 | 5 | 110088850 | T | C | rs10515411 |
| 696 | 5 | 110161259 | T | G | rs4460176 |
| 697 | 5 | 113890004 | T | C | rs1482377 |
| 698 | 5 | 117146494 | T | C | rs7710231 |
| 699 | 5 | 119788359 | C | A | rs2973940 |
| 700 | 5 | 120894485 | T | C | rs9327172 |
| 70 | 5 | 121469167 | A | G | rs6874603 |
| 702 | 5 | 123742189 | C | T | rs17150698 |
| 703 | 5 | 123801665 | T | C | rs407364 |
| 704 | 5 | 123803526 | A | G | rs154293 |
| 705 | 5 | 123868698 | T | G | rs330676 |
| 706 | 5 | 129274581 | G | C | rs10064168 |
| 707 | 5 | 129292101 | A | C | rs252668 |
| 708 | 5 | 129292600 | C | G | rs252670 |
| 709 | 5 | 136623362 | T | C | rs10051626 |
| 710 | 5 | 143979881 | G | A | rs160870 |
| 711 | 5 | 153341040 | G | A | rs11167629 |
| 712 | 5 | 161046366 | G | A | rs4921175 |
| 713 | 5 | 161092424 | G | C | rs1387612 |
| 714 | 5 | 161092556 | G | A | rs1387611 |
| 715 | 5 | 162282821 | A | G | rs17060201 |
| 716 | 5 | 163990363 | T | G | rs6860898 |
| 717 | 5 | 166256592 | G | A | rs13163863 |
| 718 | 5 | 166520830 | G | A | rs2961857 |
| 719 | 5 | 166639250 | T | G | rs347993 |
| 720 | 5 | 171612618 | G | A | rs2879407 |
| 721 | 5 | 175097385 | A | G | rs12109464 |
| 722 | 5 | 175131495 | A | G | rs2248804 |
| 723 | 5 | 175175435 | C | A | rs13169756 |
| 724 | 5 | 175180838 | A | G | rs695221 |
| 725 | 6 | 9398662 | C | T | rs214444 |
| 726 | 6 | 15765518 | G | T | rs413080 |
| 727 | 6 | 23229496 | G | C | rs13208665 |
| 728 | 6 | 23506262 | A | G | rs199108 |
| 729 | 6 | 40101697 | G | T | rs3008841 |
| 730 | 6 | 48314914 | T | G | rs614819 |
| 731 | 6 | 48447416 | G | A | rs9395354 |
| 732 | 6 | 50290485 | C | T | rs1361504 |
| 733 | 6 | 51015810 | G | A | rs9357680 |
| 734 | 6 | 51258411 | A | T | rs2465049 |
| 735 | 6 | 51260442 | C | T | rs2465034 |
| 736 | 6 | 61063311 | T | C | rs3863232 |
| 737 | 6 | 66291673 | A | C | rs2188585 |
| 738 | 6 | 66320742 | G | T | rs6455096 |
| 739 | 6 | 66837013 | A | G | rs4395708 |
| 740 | 6 | 67314965 | C | T | rs9363740 |
| 741 | 6 | 69490352 | G | A | rs1831196 |
| 742 | 6 | 76194419 | A | G | rs4467744 |
| 743 | 6 | 78709713 | A | G | rs10485127 |
| 744 | 6 | 80660369 | G | A | rs2061044 |
| 745 | 6 | 82770749 | A | G | rs4111785 |
| 746 | 6 | 86541476 | A | G | rs952571 |
| 747 | 6 | 90688053 | T | A | rs8180550 |
| 748 | 6 | 90781546 | G | C | rs1145757 |
| 749 | 6 | 90808976 | T | C | rs4707649 |
| 750 | 6 | 90811724 | G | C | rs1394248 |
| 751 | 6 | 90929095 | A | G | rs1016075 |
| 752 | 6 | 90929121 | A | T | rs1016074 |
| 753 | 6 | 90929604 | C | T | rs1145807 |
| 754 | 6 | 90988142 | T | G | rs9353772 |
| 755 | 6 | 91083391 | C | T | rs10944520 |
| 756 | 6 | 91130011 | A | G | rs9362831 |
| 757 | 6 | 91132198 | G | A | rs4128787 |
| 758 | 6 | 91134791 | C | T | rs9345098 |
| 759 | 6 | 94548200 | A | G | rs2208173 |
| 760 | 6 | 94712496 | T | G | rs6911710 |
| 761 | 6 | 95388320 | G | A | rs2890370 |
| 762 | 6 | 98512999 | G | A | rs150394 |
| 763 | 6 | 99749409 | G | A | rs7742890 |
| 764 | 6 | 102190424 | G | C | rs6902587 |
| 765 | 6 | 102580272 | T | C | rs7758751 |
| 766 | 6 | 102853749 | C | T | rs1431222 |
| 767 | 6 | 103705995 | A | T | rs4945935 |
| 768 | 6 | 104288295 | G | A | rs201204 |
| 769 | 6 | 112723118 | C | T | rs6920975 |
| 770 | 6 | 115749483 | T | C | rs9320547 |
| 771 | 6 | 119496835 | T | C | rs9374799 |
| 772 | 6 | 119563003 | G | A | rs1499564 |
| 773 | 6 | 120117704 | G | T | rs1475164 |
| 774 | 6 | 120188045 | T | C | rs155162 |
| 775 | 6 | 121961831 | A | G | rs1521216 |
| 776 | 6 | 122050973 | C | A | rs9372675 |
| 777 | 6 | 137328957 | C | T | rs1145965 |
| 778 | 6 | 137367252 | C | T | rs12212642 |
| 779 | 6 | 140320875 | T | C | rs12055615 |
| 780 | 6 | 141121688 | A | G | rs4385332 |
| 781 | 6 | 141132008 | T | C | rs7738388 |
| 782 | 6 | 141848344 | C | T | rs9389952 |
| 783 | 6 | 144959017 | G | A | rs9390266 |
| 784 | 6 | 145049586 | T | C | rs2153143 |
| 785 | 6 | 153788411 | A | G | rs12213446 |
| 786 | 6 | 155755673 | C | T | rs2353784 |
| 787 | 6 | 164449032 | A | G | rs9347854 |
| 788 | 6 | 164553942 | A | G | rs9459136 |
| 789 | 6 | 164564773 | G | A | rs4709863 |
| 790 | 6 | 164642176 | G | T | rs8180617 |
| 791 | 7 | 9344656 | T | A | rs12531647 |
| 792 | 7 | 9348488 | A | G | rs17161591 |
| 793 | 7 | 9494494 | T | C | rs2350850 |
| 794 | 7 | 10342422 | T | C | rs6415296 |
| 795 | 7 | 19248756 | T | C | rs17140993 |
| 796 | 7 | 41233817 | A | G | rs2051870 |
| 797 | 7 | 41274867 | C | T | rs417916 |
| 798 | 7 | 41275609 | T | A | rs452475 |
| 799 | 7 | 41377410 | T | G | rs12701888 |
| 800 | 7 | 41377669 | C | T | rs10267966 |
| 801 | 7 | 41379974 | C | T | rs273085 |
| 802 | 7 | 41470579 | T | A | rs273146 |
| 803 | 7 | 42388980 | C | T | rs12530988 |
| 804 | 7 | 42456896 | A | C | rs1123227 |
| 805 | 7 | 42457429 | T | C | rs12673844 |
| 806 | 7 | 49029150 | T | C | rs12718319 |
| 807 | 7 | 49114242 | G | T | rs4486140 |
| 808 | 7 | 49360186 | G | A | rs10227315 |
| 809 | 7 | 51967064 | T | C | rs453828 |
| 810 | 7 | 52372127 | G | A | rs3735071 |
| 811 | 7 | 52720540 | T | C | rs9690428 |
| 812 | 7 | 52726918 | T | C | rs1528995 |
| 813 | 7 | 54080254 | A | G | rs12540328 |
| 814 | 7 | 61089312 | G | A | rs13247259 |
| 815 | 7 | 67472597 | G | C | rs11978872 |
| 816 | 7 | 67901460 | G | T | rs10240492 |
| 817 | 7 | 68477082 | T | A | rs6944505 |
| 818 | 7 | 68851831 | T | A | rs17670760 |
| 819 | 7 | 71008019 | C | T | rs1525293 |
| 820 | 7 | 81075164 | C | T | rs17154740 |
| 821 | 7 | 83765933 | A | G | rs10249800 |
| 822 | 7 | 83848354 | C | T | rs10487868 |
| 823 | 7 | 84687890 | G | A | rs4278108 |
| 824 | 7 | 86322767 | G | A | rs6977312 |
| 825 | 7 | 97307465 | G | A | rs2394621 |
| 826 | 7 | 109098880 | A | G | rs7806371 |
| 827 | 7 | 113314158 | G | A | rs10240906 |
| 828 | 7 | 113596455 | T | A | rs2462671 |
| 829 | 7 | 113653136 | A | G | rs6962827 |
| 830 | 7 | 114796071 | T | C | rs4366023 |
| 831 | 7 | 115334141 | T | A | rs7777244 |
| 832 | 7 | 126066219 | C | T | rs6958891 |
| 833 | 7 | 145433787 | A | G | rs850398 |
| 834 | 7 | 153040171 | T | C | rs6464300 |
| 835 | 7 | 153245595 | C | T | rs1882828 |
| 836 | 8 | 5242887 | G | C | rs6558984 |
| 837 | 8 | 20800719 | C | T | rs7000415 |
| 838 | 8 | 34451965 | A | G | rs958085 |
| 839 | 8 | 34666892 | G | A | rs7464104 |
| 840 | 8 | 34666961 | T | C | rs4455823 |
| 841 | 8 | 41137620 | C | T | rs4736795 |
| 842 | 8 | 41139405 | A | G | rs13273599 |
| 843 | 8 | 41139982 | G | C | rs4736924 |
| 844 | 8 | 51115453 | A | G | rs7824553 |
| 845 | 8 | 59239001 | C | T | rs4237030 |
| 846 | 8 | 77126826 | C | T | rs10095813 |
| 847 | 8 | 77639021 | T | C | rs748 |
| 848 | 8 | 83525360 | C | A | rs4418335 |
| 849 | 8 | 88994211 | G | A | rs1994441 |
| 850 | 8 | 97462688 | A | G | rs6995779 |
| 851 | 8 | 97507691 | G | A | rs11784202 |
| 852 | 8 | 106874764 | T | C | rs7827343 |
| 853 | 8 | 107748427 | A | G | rs7005234 |
| 854 | 8 | 110219023 | T | C | rs2350886 |
| 855 | 8 | 113719319 | C | T | rs1377226 |
| 856 | 8 | 113778550 | T | C | rs7000988 |
| 857 | 8 | 115236105 | A | T | rs2980415 |
| 858 | 8 | 115237679 | G | A | rs12677395 |
| 859 | 8 | 117650318 | T | C | rs1508569 |
| 860 | 8 | 121223780 | C | T | rs12542986 |
| 861 | 8 | 122302054 | C | T | rs7012904 |
| 862 | 8 | 131608452 | A | G | rs277037 |
| 863 | 8 | 137215832 | A | C | rs4909357 |
| 864 | 8 | 139229983 | C | T | rs12543243 |
| 865 | 8 | 139285847 | C | G | rs7829324 |
| 866 | 8 | 139324635 | C | A | rs4736071 |
| 867 | 8 | 139342742 | C | T | rs4736253 |
| 868 | 8 | 139345969 | A | G | rs6980509 |
| 869 | 9 | 1436134 | T | C | rs876977 |
| 870 | 9 | 1654460 | T | C | rs771901 |
| 871 | 9 | 8090484 | T | C | rs12237393 |
| 872 | 9 | 11129139 | T | C | rs10809353 |
| 873 | 9 | 12402842 | A | G | rs2773858 |
| 874 | 9 | 20096360 | C | T | rs10757118 |
| 875 | 9 | 20178682 | C | T | rs1413254 |
| 876 | 9 | 24298510 | A | C | rs6475790 |
| 877 | 9 | 24707196 | C | T | rs16908722 |
| 878 | 9 | 24717138 | T | A | rs16908747 |
| 879 | 9 | 24750181 | T | G | rs7849050 |
| 880 | 9 | 24769580 | A | C | rs4319187 |
| 881 | 9 | 25212651 | G | A | rs1412517 |
| 882 | 9 | 25297842 | C | A | rs1156347 |
| 883 | 9 | 26222210 | G | A | rs1369203 |
| 884 | 9 | 26333944 | T | C | rs1336466 |
| 885 | 9 | 26359487 | C | T | rs915508 |
| 886 | 9 | 26457172 | T | C | rs1328425 |
| 887 | 9 | 27723715 | G | A | rs2026147 |
| 888 | 9 | 27724118 | G | A | rs13286192 |
| 889 | 9 | 29022154 | C | T | rs10968887 |
| 890 | 9 | 29434259 | T | A | rs1888817 |
| 891 | 9 | 31837336 | A | G | rs10511887 |
| 892 | 9 | 31873013 | G | A | rs943926 |
| 893 | 9 | 31895375 | A | G | rs10970585 |
| 894 | 9 | 38250852 | G | A | rs12337623 |
| 895 | 9 | 73280787 | T | C | rs10125086 |
| 896 | 9 | 74109040 | A | G | rs10869324 |
| 897 | 9 | 74172675 | A | G | rs966266 |
| 898 | 9 | 78467599 | C | T | rs12351660 |
| 899 | 9 | 78863824 | C | A | rs6559423 |
| 900 | 9 | 78893663 | G | A | rs4877311 |
| 901 | 9 | 78900517 | C | T | rs1347815 |
| 902 | 9 | 80144393 | G | A | rs10512097 |
| 903 | 9 | 80223270 | C | A | rs617941 |
| 904 | 9 | 80972229 | G | T | rs10780476 |
| 905 | 9 | 81175482 | C | T | rs2777678 |
| 906 | 9 | 103799644 | A | C | rs988692 |
| 907 | 9 | 103801174 | G | T | rs7046401 |
| 908 | 9 | 108510416 | C | A | rs28615905 |
| 909 | 9 | 118200004 | A | G | rs12551731 |
| 910 | 9 | 118248454 | G | A | rs2149010 |
| 911 | 9 | 118315750 | A | G | rs10124323 |
| 912 | 9 | 118319036 | C | T | rs7047454 |
| 913 | 9 | 118559223 | A | G | rs7036491 |
| 914 | 9 | 118563946 | G | A | rs4294251 |
| 915 | 9 | 118870424 | T | C | rs4145935 |
| 916 | 9 | 119680227 | A | G | rs6415829 |
| 917 | 9 | 120076996 | G | A | rs7870808 |
| 918 | 9 | 124137173 | G | A | rs2638384 |

### 2-1: Discovery of 482 SNP Markers

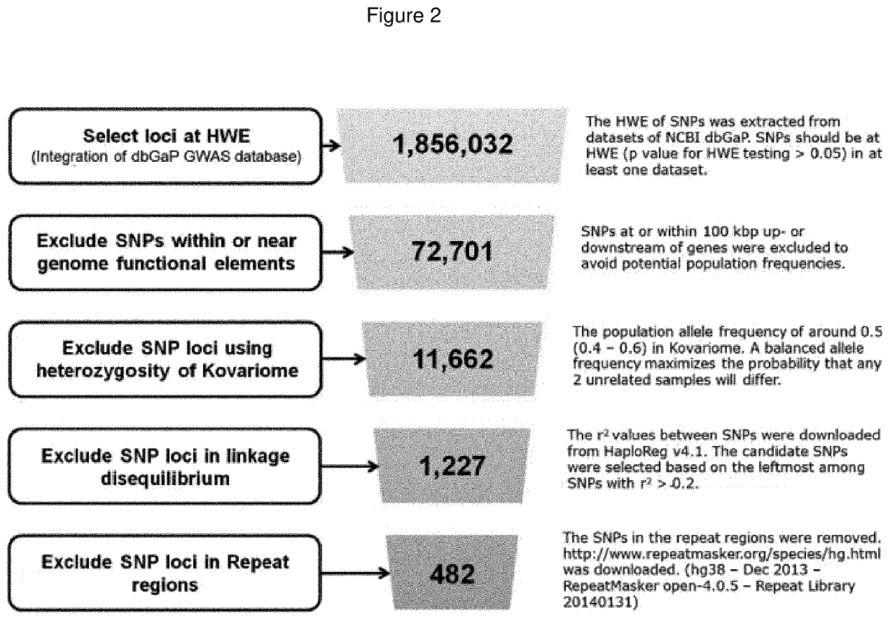

SNP markers were discovered following the same process in Example 2-1 described above and following the sequence shown in the schematic diagram in FIG. 2, except that to further reduce the number of SNP markers from the 918 SNP markers extracted above, only those SNPs having an allele frequency of about 0.4 to 0.6 in Korean populations were extracted. As a result, finally 482 SNP markers were discovered. The 918 SNPs discovered in Example 2-1 above and the 482 SNPs discovered in Example 2-2 here are different from each other.

More specifically as shown in FIG. 2, 1,856,032 SNPs were discovered as a result of selecting loci at HWE, 72,701 SNPs were discovered as a result of excluding SNPs within or near genomic regions, 11,662 SNPs were discovered as a result of excluding SNP loci using heterozygosity of Kovariome, 1,227 SNPs were discovered as a result of excluding SNP loci in linkage disequilibrium, and as a result of excluding SNP loci in repeated regions therefrom, finally a panel of 482 SNPs were discovered.

482 SNPs selected for kinship identification in Korean are shown in Table 2 below, and sequences, each consisting of 201 nucleotides including each SNP at position 101, are shown in SEQ ID NO: 919 to SEQ ID NO: 1400. The position of the SNP on the chromosome is expressed with respect to human reference genome GRCh38 (hg38), and each SNP may have reference allele (ref. allele) or an alternative allele (alt. allele) positioned at position 101 [n]. In addition, reference SNP cluster ID numbers (rs #) of dbSNP, which is an open database provided by NCBI, were cited together.

TABLE 2

| SEQ ID | | Position | Ref. | Alt. | |
|---|---|---|---|---|---|
| NO: | Chromosome # | (GRCh38) | Allele | Allele | dbSNP rs# |
| 919 | 1 | 29604465 | C | T | rs10914306 |
| 920 | 1 | 30527344 | G | A | rs385109 |
| 921 | 1 | 30587633 | A | C | rs472061 |
| 922 | 1 | 34488255 | T | G | rs4653045 |
| 923 | 1 | 34522675 | A | T | rs7556065 |
| 924 | 1 | 79684353 | A | T | rs1479739 |
| 925 | 1 | 79770134 | T | C | rs12022561 |
| 926 | 1 | 80793258 | C | T | rs2147084 |
| 927 | 1 | 98389610 | G | A | rs728656 |
| 928 | 1 | 98527441 | G | A | rs1081415 |
| 929 | 1 | 101495586 | T | G | rs4907942 |
| 930 | 1 | 101509262 | T | C | rs187823 |
| 931 | 1 | 102097084 | C | A | rs6684718 |
| 932 | 1 | 102489787 | A | G | rs1934710 |
| 933 | 1 | 104435454 | C | T | rs994637 |
| 934 | 1 | 104460389 | A | C | rs7417401 |
| 935 | 1 | 105132509 | C | A | rs6700492 |
| 936 | 1 | 105135344 | T | C | rs4399210 |
| 937 | 1 | 105175009 | A | G | rs4847034 |
| 938 | 1 | 106256205 | G | C | rs10881377 |
| 939 | 1 | 106648863 | A | G | rs10785799 |
| 940 | 1 | 118369123 | T | C | rs7541204 |
| 941 | 1 | 118450338 | G | A | rs2798449 |
| 942 | 1 | 163617565 | G | T | rs10917813 |
| 943 | 1 | 164034133 | G | A | rs2346420 |
| 944 | 1 | 187817530 | G | T | rs2430184 |
| 945 | 1 | 187853414 | G | C | rs2430147 |
| 946 | 1 | 191496601 | G | C | rs1338033 |
| 947 | 1 | 191560373 | T | A | rs1113634 |
| 948 | 1 | 195163463 | C | T | rs598224 |
| 949 | 1 | 195871676 | A | G | rs4256795 |
| 950 | 1 | 208472915 | T | C | rs12024584 |
| 951 | 1 | 208868883 | G | A | rs6692199 |
| 952 | 1 | 214768252 | G | A | rs12022703 |
| 953 | 1 | 217238392 | G | A | rs7542498 |
| 954 | 1 | 218654513 | T | C | rs1481345 |
| 955 | 1 | 218688412 | T | A | rs1383759 |
| 956 | 1 | 218765222 | C | T | rs9431104 |
| 957 | 1 | 221010746 | T | C | rs1360887 |
| 958 | 1 | 238648216 | G | A | rs12729311 |
| 959 | 10 | 9564609 | C | T | rs1324884 |
| 960 | 10 | 9595693 | T | A | rs11256268 |
| 961 | 10 | 20655689 | G | T | rs11012237 |
| 962 | 10 | 29068578 | A | G | rs12767581 |
| 963 | 10 | 29085839 | C | T | rs517770 |
| 964 | 10 | 29103651 | C | T | rs540804 |
| 965 | 10 | 57615107 | C | T | rs4463781 |
| 966 | 10 | 61227405 | G | A | rs10821875 |
| 967 | 10 | 81366347 | C | T | rs561182 |
| 968 | 10 | 81574714 | A | G | rs11190474 |
| 969 | 10 | 83556706 | T | A | rs7081137 |
| 970 | 10 | 84834585 | G | A | rs2991926 |
| 971 | 10 | 109054770 | T | C | rs723483 |
| 972 | 10 | 125292604 | A | G | rs3851566 |
| 973 | 10 | 128431579 | T | G | rs12782052 |
| 974 | 10 | 128549205 | A | G | rs12413518 |
| 975 | 10 | 128800718 | G | A | rs12783941 |
| 976 | 11 | 26825763 | C | T | rs1375971 |
| 977 | 11 | 36877200 | C | T | rs7120395 |
| 978 | 11 | 36977052 | A | G | rs11033894 |
| 979 | 11 | 39379076 | A | G | rs994587 |
| 980 | 11 | 39677236 | T | C | rs11035405 |
| 981 | 11 | 42834470 | C | T | rs7118693 |
| 982 | 11 | 42835318 | C | T | rs6485361 |
| 983 | 11 | 48744042 | A | C | rs10769445 |
| 984 | 11 | 91521201 | G | A | rs4753336 |
| 985 | 11 | 98834858 | C | T | rs7925701 |
| 986 | 11 | 100459226 | C | T | rs11224242 |
| 987 | 11 | 113586326 | T | C | rs10750026 |
| 988 | 11 | 114823551 | C | T | rs12279327 |
| 989 | 11 | 114990330 | G | A | rs7944043 |
| 990 | 11 | 116057018 | T | A | rs11826183 |
| 991 | 11 | 116126245 | C | T | rs489357 |
| 992 | 11 | 116134453 | T | C | rs576425 |
| 993 | 11 | 127508316 | G | T | rs10790898 |
| 994 | 11 | 127522266 | G | C | rs17187 |
| 995 | 11 | 127787176 | A | G | rs10790919 |
| 996 | 12 | 29886091 | G | A | rs12302114 |
| 997 | 12 | 37727134 | A | G | rs1663281 |
| 998 | 12 | 69101047 | T | G | rs7397436 |
| 999 | 12 | 72873355 | C | T | rs7962306 |
| 1000 | 12 | 74909600 | A | C | rs6582255 |
| 1001 | 12 | 82112924 | C | G | rs12829083 |
| 1002 | 12 | 82115234 | G | A | rs7968088 |
| 1003 | 12 | 84558692 | T | A | rs10779106 |
| 1004 | 12 | 87493362 | G | A | rs1603406 |
| 1005 | 13 | 23033352 | C | A | rs9552805 |
| 1006 | 13 | 31588766 | A | G | rs202451 |
| 1007 | 13 | 34807654 | T | G | rs13378363 |
| 1008 | 13 | 37370702 | G | A | rs9594199 |
| 1009 | 13 | 47110259 | G | C | rs9567787 |
| 1010 | 13 | 55866632 | T | C | rs2645931 |
| 1011 | 13 | 56726154 | A | G | rs812612 |
| 1012 | 13 | 56990794 | T | C | rs7995965 |
| 1013 | 13 | 56991225 | A | G | rs7995278 |
| 1014 | 13 | 64671768 | G | T | rs359365 |
| 1015 | 13 | 64768896 | C | T | rs9540214 |
| 1016 | 13 | 69512112 | T | C | rs9572181 |
| 1017 | 13 | 70307833 | T | C | rs9592695 |
| 1018 | 13 | 72216211 | A | G | rs488071 |
| 1019 | 13 | 84680934 | T | C | rs4284530 |
| 1020 | 13 | 86567392 | T | C | rs8000086 |
| 1021 | 13 | 103176202 | G | A | rs7990727 |
| 1022 | 13 | 103176464 | C | T | rs1750410 |
| 1023 | 13 | 103176493 | G | A | rs1730643 |
| 1024 | 13 | 103226212 | A | G | rs6491731 |
| 1025 | 13 | 103545411 | A | G | rs2390905 |
| 1026 | 13 | 103617386 | T | C | rs996758 |
| 1027 | 13 | 103720841 | T | C | rs2065391 |
| 1028 | 13 | 104362873 | G | A | rs1590919 |
| 1029 | 13 | 104522717 | A | C | rs9300939 |
| 1030 | 13 | 105067040 | T | C | rs9519577 |
| 1031 | 13 | 105235992 | G | A | rs3015358 |
| 1032 | 14 | 43833881 | G | C | rs941943 |
| 1033 | 14 | 62322229 | G | T | rs8012287 |
| 1034 | 14 | 82897096 | A | G | rs6574729 |
| 1035 | 14 | 83205008 | G | A | rs8013139 |
| 1036 | 14 | 83376648 | T | G | rs229807 |
| 1037 | 14 | 84313150 | C | T | rs10148642 |
| 1038 | 14 | 86616242 | C | T | rs6574903 |
| 1039 | 15 | 37678161 | G | A | rs8038727 |
| 1040 | 15 | 53255483 | G | A | rs4776123 |
| 1041 | 15 | 96954277 | T | G | rs6496198 |
| 1042 | 15 | 96977000 | T | C | rs12900387 |
| 1043 | 16 | 17642369 | T | C | rs9923864 |
| 1044 | 16 | 26929850 | G | A | rs11074808 |
| 1045 | 16 | 26938513 | T | C | rs4787393 |
| 1046 | 16 | 26949195 | C | G | rs9929685 |
| 1047 | 16 | 48961634 | G | C | rs12598934 |
| 1048 | 16 | 51903322 | T | C | rs2386959 |
| 1049 | 16 | 59318853 | T | A | rs2081227 |
| 1050 | 16 | 60193214 | G | T | rs12598588 |
| 1051 | 16 | 61421425 | G | C | rs4784117 |
| 1052 | 16 | 79387940 | G | T | rs12444602 |
| 1053 | 16 | 79927433 | A | G | rs4889046 |
| 1054 | 16 | 82321149 | G | A | rs2967364 |
| 1055 | 17 | 10982759 | C | G | rs4522450 |
| 1056 | 17 | 11025731 | C | T | rs7211507 |
| 1057 | 17 | 14523242 | C | G | rs11078254 |
| 1058 | 17 | 52262307 | G | C | rs1486751 |
| 1059 | 17 | 52270460 | C | G | rs7220900 |
| 1060 | 17 | 65347054 | T | C | rs6504295 |
| 1061 | 17 | 70885429 | T | C | rs2367119 |
| 1062 | 18 | 4571090 | C | A | rs7230421 |
| 1063 | 18 | 28348700 | C | T | rs7237664 |
| 1064 | 18 | 30562573 | T | C | rs2617892 |
| 1065 | 18 | 38440132 | C | T | rs7241992 |
| 1066 | 18 | 38461161 | T | C | rs7232769 |
| 1067 | 18 | 38482597 | G | A | rs9947632 |
| 1068 | 18 | 38525993 | C | T | rs12965155 |
| 1069 | 18 | 41139108 | C | T | rs17696192 |
| 1070 | 18 | 43634254 | C | T | rs12455868 |
| 1071 | 18 | 56461832 | C | T | rs374277 |
| 1072 | 18 | 61158634 | T | G | rs242717 |
| 1073 | 18 | 66722766 | A | G | rs11659281 |
| 1074 | 18 | 72232917 | G | A | rs8084367 |
| 1075 | 18 | 72383000 | A | G | rs7505475 |
| 1076 | 18 | 73006310 | A | C | rs11151820 |
| 1077 | 18 | 73024567 | C | T | rs1114342 |
| 1078 | 19 | 24338821 | G | A | rs3976345 |
| 1079 | 2 | 4375557 | C | G | rs12052610 |
| 1080 | 2 | 5054916 | A | G | rs6714934 |
| 1081 | 2 | 5135113 | T | C | rs1453760 |
| 1082 | 2 | 5205599 | A | G | rs4462764 |
| 1083 | 2 | 5431227 | A | G | rs13412303 |
| 1084 | 2 | 35575230 | C | T | rs11678496 |
| 1085 | 2 | 35897209 | C | T | rs1524144 |
| 1086 | 2 | 35897322 | G | T | rs1524145 |
| 1087 | 2 | 35909964 | C | T | rs12712470 |
| 1088 | 2 | 36152577 | G | A | rs1167454 |
| 1089 | 2 | 40879880 | G | A | rs982428 |
| 1090 | 2 | 49709726 | A | G | rs1914778 |
| 1091 | 2 | 53281551 | T | G | rs11678259 |
| 1092 | 2 | 56532828 | G | C | rs1304898 |
| 1093 | 2 | 57645567 | C | T | rs2612313 |
| 1094 | 2 | 75355062 | T | C | rs4853141 |
| 1095 | 2 | 78710839 | G | A | rs1993146 |
| 1096 | 2 | 81057349 | C | A | rs7597163 |
| 1097 | 2 | 81807528 | C | G | rs4852629 |
| 1098 | 2 | 83395037 | C | T | rs2043015 |
| 1099 | 2 | 83761345 | T | G | rs1115900 |
| 1100 | 2 | 117299195 | T | C | rs1377407 |
| 1101 | 2 | 118347401 | G | A | rs4276037 |
| 1102 | 2 | 123584690 | G | A | rs6709442 |
| 1103 | 2 | 125967099 | T | G | rs6719448 |
| 1104 | 2 | 129702523 | T | G | rs4662980 |
| 1105 | 2 | 133703450 | A | G | rs4305321 |
| 1106 | 2 | 133753999 | G | A | rs1900351 |
| 1107 | 2 | 133771383 | T | C | rs746838 |
| 1108 | 2 | 133826291 | G | C | rs4954082 |
| 1109 | 2 | 133868385 | T | C | rs10173030 |
| 1110 | 2 | 133893655 | G | A | rs12464286 |
| 1111 | 2 | 133896773 | T | G | rs1838883 |
| 1112 | 2 | 137827035 | C | T | rs733196 |
| 1113 | 2 | 142309589 | A | G | rs6429959 |
| 1114 | 2 | 146295728 | G | T | rs2381913 |
| 1115 | 2 | 146707627 | G | A | rs10184858 |
| 1116 | 2 | 150761094 | G | A | rs11893757 |
| 1117 | 2 | 153030496 | T | C | rs12618138 |
| 1118 | 2 | 160616670 | G | C | rs13024212 |
| 1119 | 2 | 180292263 | C | T | rs3845718 |
| 1120 | 2 | 191570538 | G | A | rs4640333 |
| 1121 | 2 | 192471682 | T | C | rs1601323 |
| 1122 | 2 | 192941098 | T | C | rs765278 |
| 1123 | 2 | 193464423 | T | C | rs764142 |
| 1124 | 2 | 195136595 | A | G | rs938065 |
| 1125 | 2 | 220959282 | T | C | rs13021771 |
| 1126 | 2 | 220987575 | A | G | rs2034747 |
| 1127 | 2 | 221759957 | A | G | rs1364386 |
| 1128 | 2 | 225864180 | C | T | rs13013295 |
| 1129 | 2 | 226293522 | A | G | rs1515113 |
| 1130 | 2 | 226466009 | A | T | rs6724846 |
| 1131 | 2 | 235295103 | T | G | rs11901273 |
| 1132 | 20 | 6891490 | C | G | rs6054584 |
| 1133 | 20 | 12711853 | A | G | rs6033500 |
| 1134 | 20 | 40229271 | G | C | rs6028968 |
| 1135 | 20 | 40515646 | T | A | rs6129620 |
| 1136 | 20 | 41854780 | T | C | rs2211350 |
| 1137 | 20 | 41886176 | T | C | rs7265011 |
| 1138 | 20 | 54373070 | C | T | rs6023167 |
| 1139 | 20 | 54963579 | A | G | rs2870375 |
| 1140 | 20 | 60916617 | A | C | rs2426956 |
| 1141 | 21 | 20045854 | A | G | rs8126652 |
| 1142 | 21 | 23594494 | T | C | rs12626525 |
| 1143 | 21 | 26731325 | A | G | rs2187226 |
| 1144 | 3 | 5358553 | G | C | rs4685910 |
| 1145 | 3 | 5378490 | A | G | rs7649517 |
| 1146 | 3 | 5457906 | C | G | rs7649155 |
| 1147 | 3 | 5465019 | A | G | rs6775400 |
| 1148 | 3 | 5534813 | A | G | rs6774182 |
| 1149 | 3 | 5686597 | A | C | rs4685973 |
| 1150 | 3 | 5710199 | G | A | rs6799738 |
| 1151 | 3 | 5782842 | A | G | rs1435352 |
| 1152 | 3 | 16071585 | C | A | rs11711890 |
| 1153 | 3 | 26501472 | A | C | rs7637842 |
| 1154 | 3 | 26862402 | C | T | rs11711747 |
| 1155 | 3 | 30169615 | T | C | rs2371997 |
| 1156 | 3 | 31025960 | C | T | rs294314 |
| 1157 | 3 | 35043475 | T | C | rs713144 |
| 1158 | 3 | 67111680 | C | T | rs7632236 |
| 1159 | 3 | 84204191 | G | A | rs6780104 |
| 1160 | 3 | 87428480 | C | T | rs6551480 |
| 1161 | 3 | 88701707 | T | C | rs6551361 |
| 1162 | 3 | 88734991 | G | A | rs1384774 |
| 1163 | 3 | 89728859 | T | C | rs13062221 |
| 1164 | 3 | 90368610 | T | C | rs9799286 |
| 1165 | 3 | 94247621 | G | A | rs1584929 |
| 1166 | 3 | 94404708 | T | C | rs6810199 |
| 1167 | 3 | 95327126 | C | T | rs1607490 |
| 1168 | 3 | 104192865 | G | C | rs7610885 |
| 1169 | 3 | 104308959 | T | C | rs55714502 |
| 1170 | 3 | 104338639 | A | G | rs7620614 |
| 1171 | 3 | 104863022 | G | A | rs13061652 |
| 1172 | 3 | 104885661 | A | T | rs12632614 |
| 1173 | 3 | 105174140 | A | G | rs16850936 |
| 1174 | 3 | 110255073 | T | C | rs10934026 |
| 1175 | 3 | 135551769 | T | C | rs9289494 |
| 1176 | 3 | 135742813 | G | A | rs9851816 |
| 1177 | 3 | 144663747 | A | C | rs1178938 |
| 1178 | 3 | 144730082 | C | T | rs6440266 |
| 1179 | 3 | 145181942 | G | C | rs7633246 |
| 1180 | 3 | 145237602 | A | G | rs6778538 |
| 1181 | 3 | 145269689 | C | T | rs1596717 |
| 1182 | 3 | 152954990 | G | T | rs6785504 |
| 1183 | 3 | 161929653 | A | C | rs11711859 |
| 1184 | 3 | 162149732 | A | T | rs1397231 |
| 1185 | 3 | 162803551 | G | A | rs13098441 |
| 1186 | 3 | 163845479 | A | C | rs1492782 |
| 1187 | 3 | 189501341 | G | A | rs2014894 |
| 1188 | 3 | 189508343 | C | A | rs16864459 |
| 1189 | 3 | 191019468 | G | A | rs9864904 |
| 1190 | 3 | 191757874 | A | G | rs709110 |
| 1191 | 3 | 191771515 | T | A | rs1066624 |
| 1192 | 3 | 191772184 | T | C | rs1083670 |
| 1193 | 3 | 194947038 | C | T | rs2082377 |
| 1194 | 4 | 10855090 | G | A | rs9991940 |
| 1195 | 4 | 10915291 | T | C | rs12511310 |
| 1196 | 4 | 11051913 | A | G | rs6846243 |
| 1197 | 4 | 12020898 | T | G | rs6448854 |
| 1198 | 4 | 18123104 | T | C | rs6854314 |
| 1199 | 4 | 24281377 | A | G | rs6448255 |
| 1200 | 4 | 24294211 | G | A | rs4697450 |
| 1201 | 4 | 24380505 | G | C | rs2007816 |
| 1202 | 4 | 27718168 | A | G | rs1992981 |
| 1203 | 4 | 30201580 | C | T | rs2613184 |
| 1204 | 4 | 30477251 | G | A | rs4692459 |
| 1205 | 4 | 30567961 | G | A | rs10014617 |
| 1206 | 4 | 31742430 | G | A | rs1157893 |
| 1207 | 4 | 31783076 | T | G | rs988466 |
| 1208 | 4 | 32850196 | A | G | rs7438388 |
| 1209 | 4 | 34866074 | G | A | rs7670648 |
| 1210 | 4 | 35194064 | C | G | rs2170284 |
| 1211 | 4 | 35256888 | T | C | rs4541530 |
| 1212 | 4 | 35347626 | G | C | rs13112950 |
| 1213 | 4 | 35713140 | C | T | rs13126959 |
| 1214 | 4 | 36755075 | T | C | rs12650943 |
| 1215 | 4 | 45636830 | T | C | rs2061231 |
| 1216 | 4 | 45792876 | C | T | rs1389037 |
| 1217 | 4 | 59362799 | G | A | rs1119950 |
| 1218 | 4 | 60139678 | G | T | rs1345046 |
| 1219 | 4 | 60499876 | G | A | rs10780045 |
| 1220 | 4 | 60509271 | C | T | rs2340729 |
| 1221 | 4 | 63631834 | G | C | rs1456847 |
| 1222 | 4 | 63670114 | G | A | rs13103808 |
| 1223 | 4 | 64064605 | A | G | rs1449188 |
| 1224 | 4 | 66735444 | G | A | rs11937537 |
| 1225 | 4 | 67048906 | C | A | rs1425663 |
| 1226 | 4 | 91768524 | G | C | rs7435103 |
| 1227 | 4 | 95982514 | T | C | rs973852 |
| 1228 | 4 | 114389243 | G | C | rs6838486 |
| 1229 | 4 | 124944164 | A | C | rs1021631 |
| 1230 | 4 | 129489522 | A | C | rs643541 |
| 1231 | 4 | 130103316 | T | C | rs1873868 |
| 1232 | 4 | 130198459 | G | T | rs10001806 |
| 1233 | 4 | 133431748 | A | G | rs1032439 |
| 1234 | 4 | 133676682 | C | T | rs2713266 |
| 1235 | 4 | 136672250 | T | C | rs12512934 |
| 1236 | 4 | 141842417 | G | A | rs2099884 |
| 1237 | 4 | 155047285 | C | T | rs17032387 |
| 1238 | 4 | 156146376 | T | C | rs6419277 |
| 1239 | 4 | 156199668 | T | C | rs6536157 |
| 1240 | 4 | 156208487 | A | G | rs10029573 |
| 1241 | 4 | 159982164 | C | T | rs4627809 |
| 1242 | 4 | 160103006 | G | A | rs1612279 |
| 1243 | 4 | 160138722 | T | C | rs13128591 |
| 1244 | 4 | 160385012 | A | G | rs6835941 |
| 1245 | 4 | 178968553 | A | T | rs1982408 |
| 1246 | 4 | 179566153 | T | G | rs2610997 |
| 1247 | 4 | 179661345 | C | T | rs12509486 |
| 1248 | 4 | 179714973 | C | T | rs4425420 |
| 1249 | 4 | 179738934 | C | T | rs2383397 |
| 1250 | 4 | 180861421 | T | C | rs12648100 |
| 1251 | 4 | 180874878 | G | A | rs2140198 |
| 1252 | 4 | 181383720 | G | T | rs4522908 |
| 1253 | 5 | 2364512 | T | C | rs316598 |
| 1254 | 5 | 2453263 | A | G | rs1908159 |
| 1255 | 5 | 3702962 | A | C | rs1039460 |
| 1256 | 5 | 3717886 | T | G | rs1502635 |
| 1257 | 5 | 18396275 | A | C | rs4866100 |
| 1258 | 5 | 19344111 | A | G | rs16886123 |
| 1259 | 5 | 23115159 | A | C | rs2937021 |
| 1260 | 5 | 23155563 | T | G | rs7736091 |
| 1261 | 5 | 26114387 | A | G | rs7721783 |
| 1262 | 5 | 26195022 | C | T | rs4701263 |
| 1263 | 5 | 26250139 | G | A | rs6892024 |
| 1264 | 5 | 26263490 | A | T | rs4521437 |
| 1265 | 5 | 27705900 | A | G | rs11748760 |
| 1266 | 5 | 27766133 | C | T | rs12659895 |
| 1267 | 5 | 30946767 | T | G | rs1276178 |
| 1268 | 5 | 34486721 | G | A | rs10051815 |
| 1269 | 5 | 46128373 | C | T | rs11486611 |
| 1270 | 5 | 46359219 | C | T | rs4975929 |
| 1271 | 5 | 46385222 | T | A | rs12515999 |
| 1272 | 5 | 50161448 | T | C | rs8188203 |
| 1273 | 5 | 50192402 | G | A | rs7293378 |
| 1274 | 5 | 50246086 | G | A | rs11738711 |
| 1275 | 5 | 52409393 | G | A | rs10060810 |
| 1276 | 5 | 63411710 | G | A | rs4235499 |
| 1277 | 5 | 63688050 | G | T | rs13157106 |
| 1278 | 5 | 63747610 | T | C | rs260995 |
| 1279 | 5 | 63779327 | C | T | rs1472695 |
| 1280 | 5 | 63784827 | T | G | rs7706163 |
| 1281 | 5 | 71859118 | A | G | rs6878538 |
| 1282 | 5 | 84623964 | T | C | rs11960020 |
| 1283 | 5 | 84708783 | C | T | rs10462415 |
| 1284 | 5 | 84861350 | G | A | rs13165966 |
| 1285 | 5 | 84865035 | T | C | rs1500619 |
| 1286 | 5 | 99256157 | T | C | rs1116078 |
| 1287 | 5 | 103665396 | T | C | rs11952040 |
| 1288 | 5 | 110023280 | T | C | rs2925117 |
| 1289 | 5 | 119791509 | A | C | rs12521697 |
| 1290 | 5 | 119809068 | C | T | rs6595196 |
| 1291 | 5 | 120900842 | T | C | rs6888945 |
| 1292 | 5 | 123788539 | C | A | rs29614 |
| 1293 | 5 | 123801176 | C | A | rs257142 |
| 1294 | 5 | 123872869 | T | C | rs330683 |
| 1295 | 5 | 123905854 | C | T | rs12189340 |
| 1296 | 5 | 144035178 | C | T | rs316056 |
| 1297 | 5 | 144660842 | T | G | rs10075043 |
| 1298 | 5 | 153366050 | T | A | rs300340 |
| 1299 | 5 | 169401286 | A | C | rs6555860 |
| 1300 | 5 | 169414353 | G | A | rs12522101 |
| 1301 | 5 | 171558836 | T | C | rs7720865 |
| 1302 | 5 | 175157145 | C | T | rs12189524 |
| 1303 | 5 | 175184337 | T | C | rs4336372 |
| 1304 | 6 | 9411740 | A | C | rs2327167 |
| 1305 | 6 | 9492485 | A | C | rs12200413 |
| 1306 | 6 | 9495536 | C | T | rs6905187 |
| 1307 | 6 | 15789879 | G | T | rs220923 |
| 1308 | 6 | 15819154 | C | T | rs382182 |
| 1309 | 6 | 15820194 | C | T | rs522264 |
| 1310 | 6 | 61473996 | C | T | rs9360231 |
| 1311 | 6 | 62712100 | A | G | rs2843106 |
| 1312 | 6 | 62858698 | A | G | rs12524104 |
| 1313 | 6 | 62955241 | G | A | rs4710390 |
| 1314 | 6 | 66333590 | A | G | rs12530173 |
| 1315 | 6 | 66554684 | T | C | rs7752244 |
| 1316 | 6 | 66857061 | C | A | rs4593347 |
| 1317 | 6 | 66995841 | A | G | rs9453960 |
| 1318 | 6 | 76229150 | T | C | rs994107 |
| 1319 | 6 | 77204307 | G | A | rs12206214 |
| 1320 | 6 | 78038636 | G | A | rs9343715 |
| 1321 | 6 | 78342170 | T | C | rs818268 |
| 1322 | 6 | 90853774 | A | C | rs994916 |
| 1323 | 6 | 90952546 | C | T | rs12664234 |
| 1324 | 6 | 94642318 | C | T | rs2493950 |
| 1325 | 6 | 94748599 | G | A | rs794672 |
| 1326 | 6 | 94876989 | C | T | rs197899 |
| 1327 | 6 | 95779949 | C | T | rs4840033 |
| 1328 | 6 | 103139928 | C | T | rs9373682 |
| 1329 | 6 | 112725403 | C | T | rs9384841 |
| 1330 | 6 | 114645435 | G | A | rs9481463 |
| 1331 | 6 | 115107468 | G | A | rs9488534 |
| 1332 | 6 | 115816330 | T | A | rs4113207 |
| 1333 | 6 | 117161819 | G | A | rs9400981 |
| 1334 | 6 | 137357692 | C | T | rs9373188 |
| 1335 | 6 | 137380770 | T | G | rs6913805 |
| 1336 | 6 | 145001237 | A | G | rs946317 |
| 1337 | 6 | 145040854 | A | C | rs1104835 |
| 1338 | 6 | 145045960 | C | A | rs9376894 |
| 1339 | 6 | 145081204 | A | G | rs495269 |
| 1340 | 6 | 145196271 | C | A | rs4896780 |
| 1341 | 6 | 153542928 | G | T | rs1674729 |
| 1342 | 6 | 153547060 | A | C | rs567878 |
| 1343 | 6 | 155760373 | C | T | rs11156041 |
| 1344 | 6 | 164563082 | C | G | rs2019943 |
| 1345 | 7 | 4371578 | G | A | rs10488360 |
| 1346 | 7 | 4393607 | A | G | rs6959971 |
| 1347 | 7 | 9293438 | C | G | rs4720832 |
| 1348 | 7 | 9294891 | T | A | rs4472411 |
| 1349 | 7 | 9369330 | A | G | rs7780856 |
| 1350 | 7 | 24017376 | A | C | rs10233834 |
| 1351 | 7 | 24028724 | T | C | rs11764999 |
| 1352 | 7 | 24030332 | A | G | rs719158 |
| 1353 | 7 | 41266574 | G | A | rs6943312 |
| 1354 | 7 | 41373358 | G | T | rs10251186 |
| 1355 | 7 | 41472141 | A | G | rs273148 |
| 1356 | 7 | 52603462 | A | G | rs10241795 |
| 1357 | 7 | 62559778 | A | G | rs2123573 |
| 1358 | 7 | 67485831 | G | A | rs6953207 |
| 1359 | 7 | 67908536 | T | A | rs4236217 |
| 1360 | 7 | 89645203 | T | C | rs2888705 |
| 1361 | 7 | 109106133 | T | C | rs2691991 |
| 1362 | 7 | 118715021 | G | A | rs6967747 |
| 1363 | 7 | 125751591 | A | G | rs1419607 |
| 1364 | 7 | 126201859 | C | G | rs562415 |
| 1365 | 7 | 145451172 | C | T | rs850362 |
| 1366 | 7 | 145929887 | G | A | rs4314573 |
| 1367 | 7 | 153596114 | G | A | rs4266558 |
| 1368 | 8 | 59287402 | G | C | rs4737540 |
| 1369 | 8 | 75675676 | T | G | rs724436 |
| 1370 | 8 | 92250654 | A | G | rs1444506 |
| 1371 | 8 | 97459973 | C | T | rs499177 |
| 1372 | 8 | 114054058 | C | G | rs10087835 |
| 1373 | 8 | 115087403 | T | A | rs309618 |
| 1374 | 8 | 115158348 | G | A | rs7836816 |
| 1375 | 8 | 115242924 | A | G | rs11996430 |
| 1376 | 8 | 116507424 | C | T | rs4128874 |
| 1377 | 8 | 121489749 | T | C | rs4870766 |
| 1378 | 8 | 137210744 | C | T | rs4481636 |
| 1379 | 8 | 137216529 | T | C | rs4546699 |
| 1380 | 9 | 8172467 | A | G | rs7861009 |
| 1381 | 9 | 8176622 | T | C | rs10733543 |
| 1382 | 9 | 12439164 | C | G | rs1576657 |
| 1383 | 9 | 24722712 | G | C | rs1888992 |
| 1384 | 9 | 25222108 | G | C | rs10966800 |
| 1385 | 9 | 25275215 | A | G | rs10812209 |
| 1386 | 9 | 26414782 | T | C | rs1855980 |
| 1387 | 9 | 27720612 | A | T | rs997638 |
| 1388 | 9 | 28988968 | T | C | rs10968861 |
| 1389 | 9 | 29466037 | G | A | rs2147432 |
| 1390 | 9 | 30078751 | C | G | rs2002954 |
| 1391 | 9 | 31847260 | T | C | rs10970554 |
| 1392 | 9 | 74000474 | A | T | rs2933020 |
| 1393 | 9 | 78848212 | A | G | rs10780262 |
| 1394 | 9 | 80987559 | G | T | rs10780480 |
| 1395 | 9 | 101894652 | T | C | rs823923 |
| 1396 | 9 | 118932967 | G | T | rs4631540 |
| 1397 | 9 | 119675443 | G | C | rs1860670 |
| 1398 | 9 | 119771210 | G | T | rs2781115 |
| 1399 | 9 | 120113376 | T | A | rs1335219 |
| 1400 | 9 | 124147208 | T | A | rs10986245 |

Using a panel of 482 SNPs and a panel of 918 SNPs discovered from 88 unrelated Korean individuals in Example 2 described above, in the following Examples, kinship was analyzed in a Korean population in 1-chon to 4-chon relationships based on the Korean kinship system.

As described below, genome was extracted and purified from samples obtained from a Korean population in the 1- to 4-chon relationships to produce good-quality DNA in Example 3, NGS libraries for sequencing were prepared from good-quality DNA in Example 4, and sequencing was performed using the obtained NGS libraries in Example 5. A genome alignment result file was extracted from the data produced by sequencing in Example 6, and in Example 7, based on the SNP marker information obtained in Example 2, IBS (identity by state) testing was performed from the genome alignment result file obtained in Example 6, and coefficients of relatedness were derived to perform a kinship identification analysis.

### Example 3. Sampling, and Methods of DNA Extraction, Purification and Quality Control

Genome sequencing was performed through the following separate processes: i) amplifying gDNA (genomic DNA) extracted from an oral sample by PCR (polymerase chain reaction); and ii) decoding (sequencing) the genome through NGS (next-generation sequencing) of the amplified DNA. The extracted gDNA was subjected to DNA QC (quality control) and then used to construct NGS libraries, with the objective being to secure a sufficient amount of NGS sequences.

### 3-1: Collection of Oral Samples

In the present invention, in order to derive SNP markers capable of estimating kinship even in cases where kinship estimation cannot be made by current technology because all immediate family members have died long ago or in massive disasters (that is, when there is no DNA information of parents available), or even when the genetic distance with the surviving family members is distant, the present inventors, under the permission of IRB (Institutional Review Board), collected oral samples (saliva or oral swap) from 90 Korean individuals in 1- to 4-chon relationships based on the Korean kinship system.

Among the 90 Korean individuals, 40 individuals were composed of 20 families, each containing two members who are within 2-chon relationships based on the Korean kinship system, consisting of brother-brother, sister-sister, and brother-sister, and among those 40 individuals, 8 individuals were consisted of two sets of 4 individuals in a maternal genotype second-degree relationship, in particular, mother-son-daughter-aunt relationship or mother-sisters-aunt relationship. Among the 90 Korean individuals above, 50 individuals were in 1- to 4-chon relationships based on the Korean kinship system.

3-2: gDNA Extraction

gDNA was extracted from the samples obtained in Example 3-1 by using DNeasy® Blood & Tissue Kit (Qiagen, USA) following the manufacturer's instructions. However, the method of extracting DNA from samples is not necessarily limited to the aforementioned method, and those skilled in the art would appreciate that any method of extracting gDNA known in the art may be used without limitations.

In particular, after adding 4 mL of PBS (phosphate buffered saline) to 1 mL of a saliva sample, and the resulting mixture was subjected to centrifugation for 5 minutes at 1,800×g. After the centrifugation, the supernatant was removed, and after adding 180 μl of PBS to the pellet, the pellet was resuspended, followed by addition of 20 μl of protease K and 200 μl of buffer AL (lysis buffer), and the resulting mix was thoroughly vortexed. Next, the resulting mix was incubated at 56° C. for 10 minutes before addition of 200 μl of ethanol (96% v/v to 100% v/v), and then the resulting mix was thoroughly mixed.

In order to isolate DNA from the mixture by attaching the DNA to the column, the mixture was added to a column (DNeasy Mini spin column) and was subjected to centrifugation at 6,000×g for 1 minute, and the flow-through was removed.

For washing, 500 μl of buffer AW1 (wash buffer) was added to the column and subjected to centrifugation at 6,000×g for 1 minute, and the flow-through was removed. In addition, 500 μl of buffer AW2 (wash buffer) was added to the column and subjected to centrifugation at 20,000×g for 3 minutes, and the flow-through was removed.

In order to elute DNA from the column, the column was placed in a new 1.5 mL tube and 200 μl of buffer AE (elution buffer) was directly added to the column and incubated at room temperature for 1 minute. Then, by performing centrifugation at 6,000×g for 1 minute, gDNA was obtained.

### 3-3: DNA Purification

To secure high-quality DNA, DNA clean-up was performed as follows, using AMPure XP bead (Beckman Coulter) and following the manufacturer's instructions. However, the method of DNA clean-up is not necessarily limited thereto and those skilled in the art would appreciate that any DNA clean-up method known in the art may be used without limitations.

At least 30 minutes before the experiment, magnetic beads were incubated at room temperature and thoroughly mixed before use. Beads in a volume that is 1.8 times the volume of gDNA obtained in Example 3-2 were added to the gDNA and thoroughly mixed by pipetting. Next, the resulting mixture was incubated at room temperature for 5 minutes to allow DNA and the beads to bind together.

After mild centrifugation, a sample tube was loaded in a magnetic separation rack and left for 2 minutes to 5 minutes to allow magnetic beads and DNA complexes to separate from the mixture. Once the supernatant becomes clear, the supernatant was removed.

For washing, while the sample tube is loaded in the magnetic separation rack, 200 μl of 80% v/v ethanol was added to the tube and then removed therefrom after 30 seconds. This process was repeated twice. Next, the lid of the tube was opened to let ethanol dry until before a crack forms in the sample.

The sample tube was taken out of the magnetic separation rack and combined with sterilized deionized water to elute DNA to a desired concentration. In order to separate DNA from the magnetic beads, the sample tube was incubated at room temperature for 5 minutes and reloaded in the magnetic separation rack. Once the supernatant becomes clear, by collecting the supernatant into a new tube, purified DNA was obtained.

### 3-4: DNA QC

Using a fluorometer (Qubit 4 Fluorometer) by Thermo Fisher Scientific, QC (quality control) was performed on the purified DNA obtained in Example 3-3 above, and as a result, it was confirmed that the extracted DNA was of good quality.

### Example 4. NGS Library Construction for Genome Analysis

In order to proceed the whole genome sequencing (WGS), good-quality gDNA samples passed the quality control (QC) standards in Example 3 above were used to construct libraries.

### 4-1: DNA Fragmentation and Size Selection

The good-quality DNA having passed the QC standards in Example 3 above were fragmented into 100 bp to 1000 bp, and were size selected for 300 bp to 500 bp fragments to enable construction of paired-end 150 library using bead. The size of the selected gDNA was confirmed using 2100 Bioanalyzer by Agilent, which is an automated electrophoresis tool for sample QC. In addition, the concentration was measured using Qubit™ dsDNA HS Assay kit (Thermo Fisher Scientific), which is a kit for dsDNA quantification capable of distinguishing double-stranded DNA from other nucleic acids or proteins with high sensitivity.

### 4-2: End Repair and A-Tailing

DNA fragments having a size of 300 bp to 500 bp selected in Example 4-1 were repaired by blunt ends, and A-tailing, which adds dATP (deoxyadenosine triphosphate) to the 3′ end, was performed.

### 4-3: Adaptor Ligation

Adaptors tailed with dTTP (deoxythymidine triphosphate) were ligated to ends of the DNA fragments in Example 4-2 above, and treated on a flow cell to be hybridized.

### 4-4: Library QC

The prepared libraries were checked for quality by using 2100 Bioanalyzer system by Agilent. The QC results confirmed that the libraries prepared from a total of 50 samples, namely, samples NFS_202100101 to NFS_202100116, samples NFS_202100117 to NFS_202100302, samples NFS_202100303 to NFS_202100406, and samples NFS_202100407 to NFS_202100408, were of good quality.

### 4-5: PCR Amplification

Products ligated with adapters were amplified by PCR. The final PCR products were measured for size by Agilent 2100 Bioanalyzer and were measured for concentration by the Qubit™ dsDNA HS Assay kit, and the amount (mass, ng) in the unit of nanograms that corresponds to 1 pmol (picomole) of the PCR product was calculated.

### 4-6: Single Strand Circularization

A single-strand molecule formed by heat-denaturing 1 pmol of the PCR product was ligated with a DNA ligase, and the remaining linear molecule was digested with exonuclease. Following the single strand circularization, QC was run and confirmed that the nucleic acids obtained were of good quality.

### Example 5. Method of Producing NGS Sequencing Data Using NGS Platform

Following the single strand circularization in Example 4-6 above, DNB (DNA nanoball) was prepared as described below, and DNB sequencing was conducted using high density patterned nanoarray flow cells by MGI Tech, which allow only one DNB bound per active site.

### 5-1: Preparation of DNA Nanoball

40 fmol (femtomole) of the single-strand circular DNA libraries obtained in Example 4 were hybridized with primers. After 15 minutes of RCA (rolling circle amplification) using DNB Enzyme (Phi29, ϕ29 DNA polymerase), the concentration of the libraries was measured by Qubit™ ssDNA Assay kit.

### 5-2: DNB Pooling Calculations

The sum of concentration reciprocals of the samples to be pooled, an average value thereof, and Parameter B, which is 400/average value, were calculated to confirm the pooling volume for each sample.

### 5-3: DNB Loading Preparation

Using DNB Load Buffers I and III from the DNB Rapid Reagent Kit manufactured by MGI Tech., a DNB loading mix was prepared.

### 5-4: Flow Cell Preparation and DNB Loading

In MGIDL-7 by MGI Tech, flow cells were loaded by reading the barcode of a DNB loader. The DNB loading mix prepared in Example 5-3 above was placed in DNB tube holes and flow cell loading was initiated.

### 5-5: Sequencing

A sequencing cartridge and a washing cartridge were loaded in a sequencer DNBSEQ-T7 (MGI), which was then made to recognize a flow completed with DNB loading and initiate sequencing.

### 5-6: Sequencing Results

As shown in Table 3 below, from about 50.9 billion reads (average per subject: 1.018 billion reads) of DNA extracted from 50 individuals, sequences of 7,631 Gbp (giga base pairs, one billion nucleotides) (average per subject: 152.6 Gbp) were obtained.

As indicated by Phred quality scores in Table 4 below, all of the 50 individuals generated satisfied quality score 30 (030) or higher, confirming that high-quality sequencing data were produced. Phred quality score is a quality index indicating per-base reliability and is a measure of how accurately each sequence is called.

TABLE 3
NGS data output statistics using DNBSEQ-T7

| | Raw reads | | | | |
|---|---|---|---|---|---|
| | | Total | Q20 | Q30 | Average |
| | | bases | rate | rate | depth |
| Sample ID | Total reads | (Gb) | (%) | (%) | (x) |
| NFS_202100101 | 723,482,150 | 108.5 | 96.23 | 88.88 | 35.09 |
| NFS_202100102 | 796,923,086 | 119.5 | 95.47 | 86.25 | 38.65 |
| NFS_202100103 | 787,668,392 | 118.2 | 96.08 | 87.89 | 38.20 |
| NFS_202100104 | 682,420,690 | 102.4 | 96.16 | 88.56 | 33.10 |
| NFS_202100105 | 811,409,290 | 121.7 | 96.04 | 87.84 | 39.35 |
| NFS_202100106 | 818,285,838 | 122.7 | 96.10 | 87.98 | 39.69 |
| NFS_202100107 | 886,499,748 | 133.0 | 96.09 | 88.26 | 43.00 |
| NFS_202100108 | 848,005,100 | 127.2 | 96.25 | 88.59 | 41.13 |
| NFS_202100109 | 788,801,938 | 118.3 | 96.05 | 88.89 | 38.26 |
| NFS_202100110 | 936,842,428 | 140.5 | 96.26 | 88.66 | 45.44 |
| NFS_202100111 | 1,019,239,770 | 152.9 | 96.30 | 88.84 | 49.43 |
| NFS_202100112 | 893,577,410 | 134.0 | 95.51 | 86.53 | 43.34 |
| NFS_202100113 | 754,109,606 | 113.1 | 96.01 | 88.75 | 36.57 |
| NFS_202100114 | 965,425,112 | 144.8 | 96.12 | 88.32 | 46.82 |
| NFS_202100115 | 1,020,370,606 | 153.1 | 96.09 | 88.31 | 49.49 |
| NFS_202100116 | 892,958,634 | 133.9 | 96.03 | 88.12 | 43.31 |
| NFS_202100117 | 1,238,895,246 | 185.8 | 96.43 | 89.53 | 60.09 |
| NFS_202100118 | 1,060,983,826 | 159.1 | 96.19 | 89.07 | 51.46 |
| NFS_202100119 | 968,180,426 | 145.2 | 96.21 | 89.07 | 46.96 |
| NFS_202100120 | 790,678,754 | 118.6 | 95.68 | 88.38 | 38.35 |
| NFS_202100121 | 1,130,400,008 | 169.6 | 95.78 | 88.61 | 54.82 |
| NFS_202100201 | 813,812,158 | 122.1 | 95.95 | 89.22 | 39.47 |
| NFS_202100202 | 818,128,832 | 122.7 | 95.74 | 88.76 | 39.68 |
| NFS_202100203 | 823,461,120 | 123.5 | 95.95 | 89.33 | 39.94 |
| NFS_202100204 | 812,313,488 | 121.8 | 95.99 | 89.41 | 39.40 |
| NFS_202100205 | 1,000,703,726 | 150.1 | 96.05 | 89.24 | 48.53 |
| NFS_202100206 | 1,135,314,014 | 170.3 | 96.21 | 89.84 | 55.06 |
| NFS_202100207 | 1,142,387,172 | 171.4 | 96.07 | 89.35 | 55.41 |
| NFS_202100208 | 1,247,857,738 | 187.2 | 96.24 | 89.65 | 60.52 |
| NFS_202100209 | 1,233,019,906 | 185.0 | 96.33 | 89.69 | 59.80 |
| NFS_202100301 | 985,247,144 | 147.8 | 95.79 | 88.77 | 47.78 |
| NFS_202100302 | 865,518,204 | 129.8 | 96.04 | 89.57 | 41.98 |
| NFS_202100303 | 873,348,834 | 131.0 | 96.02 | 88.92 | 42.36 |
| NFS_202100304 | 1,053,161,592 | 158.0 | 95.95 | 88.85 | 51.08 |
| NFS_202100305 | 1,186,293,470 | 177.9 | 95.89 | 88.75 | 57.54 |
| NFS_202100306 | 1,492,126,138 | 223.8 | 96.05 | 89.06 | 72.37 |
| NFS_202100307 | 851,576,702 | 127.7 | 95.94 | 88.79 | 41.30 |
| NFS_202100308 | 980,949,832 | 147.1 | 95.14 | 86.81 | 47.58 |
| NFS_202100309 | 1,090,140,206 | 163.5 | 95.89 | 88.71 | 52.87 |
| NFS_202100310 | 1,135,750,584 | 170.4 | 95.93 | 88.47 | 55.08 |
| NFS_202100311 | 995,794,578 | 149.4 | 96.00 | 88.81 | 48.30 |
| NFS_202100312 | 922,069,014 | 138.3 | 96.05 | 89.05 | 44.72 |
| NFS_202100401 | 1,176,096,778 | 176.4 | 96.14 | 89.25 | 57.04 |
| NFS_202100402 | 1,068,813,494 | 160.3 | 95.99 | 88.86 | 51.84 |
| NFS_202100403 | 1,427,382,264 | 214.1 | 96.50 | 89.88 | 69.23 |
| NFS_202100404 | 1,240,363,992 | 186.1 | 96.99 | 91.16 | 60.16 |
| NFS_202100405 | 1,020,022,412 | 153.0 | 96.87 | 90.78 | 49.47 |
| NFS_202100406 | 1,688,541,332 | 253.3 | 97.30 | 92.01 | 81.89 |
| NFS_202100407 | 1,401,333,380 | 210.2 | 97.28 | 91.70 | 67.96 |
| NFS_202100408 | 1,579,637,052 | 236.9 | 97.23 | 91.75 | 76.61 |

TABLE 4
Probability of sequencing error according to Phred quality scores

| Phred quality score | Sequencing error rate |
|---|---|
| Q10 | 10% |
| Q20 | 1% |
| Q30 | 0.1% |
| Q40 | 0.01% |

Produced were not only high-quality sequencing data but also sequences with 49.35-fold (x) average coverage depth with respect to the human reference genome (about 3.09 Gbp). This effectively shows that the entire genome was read about 49 times per subject in each sample. Accurate genome sequencing using NGS data requires the data in a volume that is 30 times greater or more than the entire genome, and the NGS data produced in the present invention was minimum 33.09 times and up to 81.89 times, confirming the accuracy of the NGS data according to the present invention. Depth of coverage refers to the average number of reads for a given nucleotide position in a particular region in the genome, and is generally expressed in the unit of x.

### Example 6. Genome Analysis

### 6-1: Sequencing Raw Data QC

For quality control of raw data generated by DNBSEQ-T7, base quality distributions were analyzed by FastQC (Andrews, 2010) and as a result, excellent base quality was confirmed.

By performing adapter trimming and quality filter analysis, sequences with adapter contamination and regions with low-quality in the sequencing data produced by NGS were removed to thereby produce high-quality sequencing data. After the adaptor trimming and quality filter analysis, it was analyzed that the depth of coverage was average 49.1× with respect to clean data. The results thereof are shown in Table 5 below.

TABLE 5
Statistics of clean-read after quality control
of sequences generated by DNBSEQ-T7

| | Clean reads | | | Clean |
|---|---|---|---|---|
| | Total | Q20 | Q30 | read |
| | bases | rate | rate | depth |
| Sample ID | (Gb) | (%) | (%) | (X) |
| NFS_202100101 | 107.7 | 96.69 | 90.10 | 34.84 |
| NFS_202100102 | 119.1 | 96.64 | 89.75 | 38.51 |
| NFS_202100103 | 117.4 | 96.80 | 90.33 | 37.97 |
| NFS_202100104 | 101.7 | 96.72 | 90.10 | 32.87 |
| NFS_202100105 | 121.1 | 96.79 | 90.31 | 39.15 |
| NFS_202100106 | 122.1 | 96.67 | 89.94 | 39.49 |
| NFS_202100107 | 132.6 | 96.79 | 90.22 | 42.86 |
| NFS_202100108 | 126.7 | 96.08 | 88.36 | 40.97 |
| NFS_202100109 | 117.9 | 96.81 | 90.36 | 38.11 |
| NFS_202100110 | 140.1 | 96.78 | 90.24 | 45.30 |
| NFS_202100111 | 152.4 | 96.94 | 90.72 | 49.28 |
| NFS_202100112 | 133.7 | 96.84 | 90.39 | 43.23 |
| NFS_202100113 | 112.7 | 96.65 | 89.96 | 36.43 |
| NFS_202100114 | 144.5 | 96.76 | 90.23 | 46.72 |
| NFS_202100115 | 152.5 | 96.63 | 89.84 | 49.30 |
| NFS_202100116 | 133.4 | 96.69 | 89.88 | 43.13 |
| NFS_202100117 | 185.0 | 96.72 | 90.11 | 59.83 |
| NFS_202100118 | 158.6 | 95.98 | 88.18 | 51.28 |
| NFS_202100119 | 144.5 | 96.34 | 89.52 | 46.71 |
| NFS_202100120 | 118.4 | 95.82 | 87.94 | 38.28 |
| NFS_202100121 | 168.7 | 96.48 | 89.95 | 54.56 |
| NFS_202100201 | 121.7 | 96.47 | 90.00 | 39.33 |
| NFS_202100202 | 122.2 | 96.12 | 88.91 | 39.51 |
| NFS_202100203 | 123.2 | 96.26 | 89.24 | 39.82 |
| NFS_202100204 | 121.2 | 96.36 | 89.59 | 39.18 |
| NFS_202100205 | 149.4 | 95.54 | 87.17 | 48.32 |
| NFS_202100206 | 169.4 | 96.32 | 89.48 | 54.77 |
| NFS_202100207 | 170.3 | 96.34 | 89.50 | 55.05 |
| NFS_202100208 | 186.4 | 96.34 | 89.62 | 60.26 |
| NFS_202100209 | 184.2 | 95.56 | 87.58 | 59.56 |
| NFS_202100301 | 147.1 | 96.83 | 90.94 | 47.55 |
| NFS_202100302 | 129.3 | 96.75 | 90.67 | 41.79 |
| NFS_202100303 | 130.7 | 96.86 | 91.07 | 42.26 |
| NFS_202100304 | 157.4 | 96.72 | 90.67 | 50.89 |
| NFS_202100305 | 177.1 | 95.57 | 87.43 | 57.27 |
| NFS_202100306 | 223.2 | 96.64 | 90.46 | 72.18 |
| NFS_202100307 | 127.4 | 97.20 | 92.23 | 41.18 |
| NFS_202100308 | 146.5 | 96.83 | 91.21 | 47.37 |
| NFS_202100309 | 162.7 | 96.67 | 90.40 | 52.62 |
| NFS_202100310 | 169.8 | 96.26 | 89.16 | 54.92 |
| NFS_202100311 | 148.9 | 96.91 | 91.20 | 48.14 |
| NFS_202100312 | 137.5 | 96.75 | 90.75 | 44.46 |
| NFS_202100401 | 175.4 | 96.71 | 90.63 | 56.71 |
| NFS_202100402 | 159.6 | 96.93 | 91.24 | 51.60 |
| NFS_202100403 | 213.5 | 96.49 | 89.98 | 69.04 |
| NFS_202100404 | 185.6 | 96.68 | 90.58 | 60.01 |
| NFS_202100405 | 152.7 | 96.83 | 90.92 | 49.36 |
| NFS_202100406 | 252.6 | 96.82 | 90.92 | 81.69 |
| NFS_202100407 | 209.6 | 96.78 | 90.82 | 67.77 |
| NFS_202100408 | 236.4 | 96.56 | 90.21 | 76.44 |

Furthermore, high-quality sequencing data was extracted also from data of 40 individuals previously produced by Nova-Seq (Illumina). The results thereof are shown in Table 6 below.

TABLE 6

| | Clean reads | | | |
|---|---|---|---|---|
| | Total bases | Q20 rate | Q30 rate | Clean read |
| Sample ID | (Gb) | (%) | (%) | depth (X) |
| 01-1 | 141.5 | 98.10 | 94.34 | 45.76 |
| 01-2 | 126.0 | 98.17 | 94.52 | 40.75 |
| 02-1 | 112.0 | 98.26 | 94.85 | 36.23 |
| 02-2 | 112.0 | 98.22 | 94.73 | 36.22 |
| 03-1 | 112.6 | 98.12 | 94.48 | 36.42 |
| 03-2 | 111.4 | 98.22 | 94.78 | 36.02 |
| 04-1 | 113.8 | 98.28 | 94.87 | 36.78 |
| 04-2 | 113.9 | 98.21 | 94.72 | 36.83 |
| 05-1 | 114.6 | 98.10 | 94.43 | 37.04 |
| 05-2 | 112.5 | 98.16 | 94.52 | 36.39 |
| 06-1 | 113.1 | 98.06 | 94.31 | 36.58 |
| 06-2 | 122.2 | 98.22 | 94.77 | 39.50 |
| 07-1 | 116.5 | 98.22 | 94.76 | 37.65 |
| 07-2 | 111.8 | 98.25 | 94.91 | 36.14 |
| 08-1 | 113.8 | 98.30 | 94.90 | 36.81 |
| 08-2 | 170.7 | 97.32 | 92.55 | 55.20 |
| 09-1 | 114.4 | 98.30 | 94.89 | 36.98 |
| 09-2 | 114.9 | 98.19 | 94.67 | 37.14 |
| 10-1 | 114.4 | 98.14 | 94.51 | 36.99 |
| 10-2 | 113.8 | 98.21 | 94.69 | 36.81 |
| 11-1 | 120.3 | 98.17 | 94.63 | 38.90 |
| 11-2 | 114.9 | 98.26 | 94.88 | 37.16 |
| 12-1 | 142.1 | 98.25 | 94.81 | 45.93 |
| 12-2 | 111.7 | 98.19 | 94.71 | 36.11 |
| 13-1 | 113.1 | 98.29 | 94.90 | 36.58 |
| 13-2 | 112.8 | 98.23 | 94.75 | 36.48 |
| 14-1 | 113.0 | 98.18 | 94.65 | 36.55 |
| 14-2 | 112.0 | 98.23 | 94.77 | 36.22 |
| 15-1 | 116.0 | 98.21 | 94.74 | 37.52 |
| 15-2 | 112.9 | 98.05 | 94.36 | 36.50 |
| 16-1 | 113.0 | 97.95 | 94.05 | 36.54 |
| 16-2 | 113.0 | 98.25 | 94.85 | 36.52 |
| 17-1 | 114.1 | 98.25 | 94.78 | 36.88 |
| 17-2 | 132.3 | 96.89 | 91.42 | 42.78 |
| 18-1 | 116.4 | 98.21 | 94.73 | 37.64 |
| 18-2 | 112.1 | 98.25 | 94.85 | 36.24 |
| 19-1 | 115.1 | 98.15 | 94.55 | 37.23 |
| 19-2 | 114.0 | 98.15 | 94.59 | 36.87 |
| 20-1 | 112.5 | 98.09 | 94.38 | 36.36 |
| 20-2 | 112.7 | 98.27 | 94.87 | 36.44 |

### 6-2: Sequence Alignment

Using clean reads after the quality control, sequence alignment was performed with the human reference genome (hg38, GRCh38) sequence using the Burrows-Wheeler Alignment (BWA) (Li H, 2013) program. The results thereof are shown in Table 7 below.

As shown in Table 7, an average alignment rate was shown to be 74.8% (alignment rate), and an average duplicate rate was shown to be 6.49%. From the alignment rate and duplicate rate, it is possible to indirectly determine authenticity of 50 individuals being sequenced and whether there is defect in sampling and library preparation.

Since most of the 50 individuals sequenced in the present invention showed relatively low alignment rates, additional testing was performed for corresponding samples, and this was incorporated in the final statistics calculated. Among those samples, it was determined to use data previously produced by Nova-Seq for two samples (NFS_202100203 and NFS_202100204), there was no additional production with regard to these two samples.

TABLE 7
Statistics of sequence alignment rate of data
generated by DNBSEQ-T7 from 50 individuals

| | Alignment | Properly | Duplicates |
|---|---|---|---|
| Sample ID | rate (%) | aligned rate (%) | rate (%) |
| NFS_202100101 | 83.82 | 82.03 | 5.15 |
| NFS_202100102 | 92.79 | 91.38 | 3.58 |
| NFS_202100103 | 84.42 | 82.83 | 5.31 |
| NFS_202100104 | 93.54 | 91.58 | 6.41 |
| NFS_202100105 | 64.39 | 63.15 | 4.84 |
| NFS_202100106 | 86.56 | 84.75 | 6.48 |
| NFS_202100107 | 80.34 | 78.79 | 4.14 |
| NFS_202100108 | 68.34 | 66.48 | 12.82 |
| NFS_202100109 | 85.1 | 83.17 | 8.17 |
| NFS_202100110 | 79.87 | 78.02 | 8.45 |
| NFS_202100111 | 68.17 | 66.31 | 8.47 |
| NFS_202100112 | 67.15 | 65.87 | 3.87 |
| NFS_202100113 | 81.03 | 78.95 | 8.58 |
| NFS_202100114 | 71.23 | 69.92 | 3.73 |
| NFS_202100115 | 66.38 | 65.17 | 4.16 |
| NFS_202100116 | 72.97 | 71.56 | 3.60 |
| NFS_202100117 | 73.23 | 72.06 | 4.54 |
| NFS_202100118 | 75.80 | 74.34 | 6.10 |
| NFS_202100119 | 86.62 | 84.82 | 4.09 |
| NFS_202100120 | 90.07 | 88.54 | 3.99 |
| NFS_202100121 | 72.54 | 71.33 | 5.04 |
| NFS_202100201 | 84.45 | 82.74 | 4.06 |
| NFS_202100202 | 83.33 | 81.5 | 7.81 |
| NFS_202100203 | 61.13 | 59.57 | 4.77 |
| NFS_202100204 | 85.27 | 83.48 | 6.11 |
| NFS_202100205 | 76.09 | 74.64 | 12.13 |
| NFS_202100206 | 70.05 | 68.8 | 34.29 |
| NFS_202100207 | 71.50 | 70.24 | 12.38 |
| NFS_202100208 | 69.22 | 68.21 | 6.68 |
| NFS_202100209 | 71.86 | 70.68 | 7.28 |
| NFS_202100301 | 74.35 | 73.18 | 4.96 |
| NFS_202100302 | 80.05 | 78.35 | 5.43 |
| NFS_202100303 | 71.02 | 69.59 | 4.97 |
| NFS_202100304 | 70.27 | 68.51 | 6.94 |
| NFS_202100305 | 69.33 | 67.87 | 5.84 |
| NFS_202100306 | 54.22 | 53.52 | 2.77 |
| NFS_202100307 | 70.49 | 69.13 | 5.56 |
| NFS_202100308 | 77.34 | 75.99 | 3.70 |
| NFS_202100309 | 84.70 | 83.34 | 4.47 |
| NFS_202100310 | 77.55 | 76.13 | 3.60 |
| NFS_202100311 | 77.23 | 76.04 | 4.30 |
| NFS_202100312 | 77.53 | 76.14 | 5.19 |
| NFS_202100401 | 57.71 | 56.41 | 5.99 |
| NFS_202100402 | 72.57 | 71.08 | 7.42 |
| NFS_202100403 | 73.26 | 71.93 | 3.22 |
| NFS_202100404 | 67.34 | 65.42 | 3.53 |
| NFS_202100405 | 73.33 | 70.59 | 4.94 |
| NFS_202100406 | 35.16 | 34.55 | 4.81 |
| NFS_202100407 | 55.87 | 54.67 | 5.43 |
| NFS_202100408 | 51.43 | 50.64 | 3.38 |

Furthermore, as shown in Table 8 below, data of 40 individuals previously generated by Nova-Seq shows an average alignment rate of 95.5%, and an average duplicates rate of 10.9%.

TABLE 8
Statistics of sequence alignment rate statistics
of data generated by Nova-Seq from 40 individuals

| Sample | Alignment | Properly aligned | Duplicates |
|---|---|---|---|
| ID | rate (%) | rate (%) | rate (%) |
| 01-1 | 82.08 | 80.45 | 11.24 |
| 01-2 | 86.74 | 85.17 | 10.6 |
| 02-1 | 98.1 | 95.82 | 10.22 |
| 02-2 | 99.68 | 97.53 | 11.21 |
| 03-1 | 96.14 | 93.86 | 11.58 |
| 03-2 | 98.62 | 94.71 | 10.62 |
| 04-1 | 97.54 | 95.52 | 11.46 |
| 04-2 | 97.98 | 96.01 | 10.47 |
| 05-1 | 93.36 | 91.26 | 10.68 |
| 05-2 | 95.85 | 94.03 | 12.06 |
| 06-1 | 99.01 | 96.89 | 10.84 |
| 06-2 | 91.99 | 87.09 | 10.35 |
| 07-1 | 98.66 | 96.04 | 11.54 |
| 07-2 | 99.53 | 95.3 | 11.36 |
| 08-1 | 91.07 | 89.35 | 11.23 |
| 08-2 | 66.66 | 64.42 | 9.99 |
| 09-1 | 94.55 | 92.96 | 11.09 |
| 09-2 | 99.23 | 96.88 | 10.38 |
| 10-1 | 99.33 | 97.28 | 11.23 |
| 10-2 | 99.17 | 97.41 | 10.56 |
| 11-1 | 99.81 | 97.85 | 12.9 |
| 11-2 | 99.7 | 97.33 | 10.37 |
| 12-1 | 81.8 | 79.61 | 10.58 |
| 12-2 | 98.01 | 95.4 | 10.03 |
| 13-1 | 97.14 | 95.5 | 11.41 |
| 13-2 | 99.43 | 97.44 | 10.25 |
| 14-1 | 99.65 | 97.38 | 11.8 |
| 14-2 | 99.66 | 97.7 | 10.73 |
| 15-1 | 97.98 | 95.56 | 10.76 |
| 15-2 | 99.66 | 96.96 | 10.7 |
| 16-1 | 98.23 | 96.1 | 10.63 |
| 16-2 | 96.29 | 93.35 | 10.74 |
| 17-1 | 91.78 | 89.81 | 11.09 |
| 17-2 | 93.57 | 91.49 | 8.97 |
| 18-1 | 98.7 | 96.66 | 12.6 |
| 18-2 | 99.25 | 96.7 | 10.1 |
| 19-1 | 93.81 | 91.88 | 10.8 |
| 19-2 | 96.12 | 93.68 | 11.05 |
| 20-1 | 98.11 | 96.42 | 11.17 |
| 20-2 | 95.13 | 92.78 | 11.06 |

### 6-3: BAM (Binary Sequence Alignment/Map) File Extraction

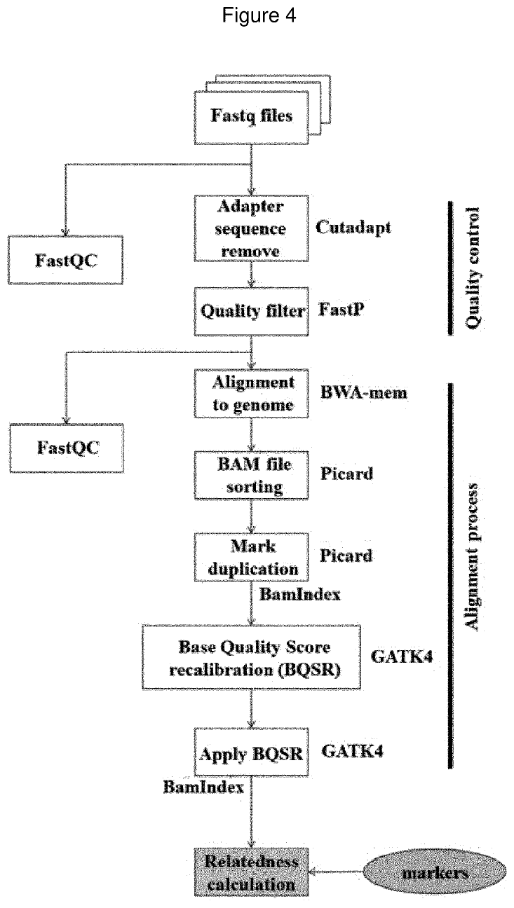

In order to use the result file (BAM format, Binary sequence Alignment/Map format) aligned with the human reference genome sequence, i) 40 individuals produced by Nova-Seq and ii) 48 individuals produced by DNBSEQ-T7 and 2 individuals produced by Nova-Seq were analyzed following the sequence shown in the pipeline schematic diagram shown in FIG. 4.

A BAM file is a file in the binary version of SAM file, and is a file format that contains alignment results of reads to the reference genome. A SAM file (Sequence Alignment/Map format File) is a TAB-delimited text format consisting of a header part and an alignment part, containing information of sequences mapped onto a particular position in the reference genome. The header part contains information about version, alignments, lengths, etc. and the alignment part contains information such as sequence information and quality information thereof. Mapping is interchangeably used to mean the same as alignment, and refers to the step of estimating a corresponding read's original position in the genome with respect to a reference genome.

### Example 7. Kinship Identification Analysis

Using the SNP panels of Example 2 described above, kinship estimation analysis was performed from the BAM file obtained as a result of the sequencing in Example 6 described above. As described in Example 2, the present inventors extracted 918 and 482 SNP markers from KoVariome, which is Korean National Standard Reference Variome database generated from 88 unrelated Korean individuals. Whether these SNP markers are useful to estimate Korean kinship among 90 Korean individuals who are in 1- to 4-chon relationships, that is, family members, was demonstrated as follows.

As shown in Table 9, first degree relatives (first-d-r) are with respect to a subject, any one of parent, brother, sister, sibling and child; second degree relatives (second-d-r) are with respect to a subject, any one of aunt, uncle, paternal aunt, grandfather, grandmother, half-brother, half-sister, and half-sibling; third degree relatives (third-d-r) are first cousins with respect to a subject; and the unrelated are none of the first-d-r, second-d-r, and third-d-r.

TABLE 9

| Distinctions | Relationship |
|---|---|
| First degree | One of parent, brother, sister, sibling, and child |
| relatives | |
| Second degree | One of aunt, uncle, paternal aunt, grandfather, grand- |
| relatives | mother, half-brother, half-sister, or half-sibling |
| Third degree | First cousins |
| relatives | |
| Unrelated | — |

Using IBS (identity by state) testing, first i) kinships among 40 individuals produced by Nova-Seq were identified, and then ii) kinship among 48 individuals produced by DNBSEQ-T7 and 2 individuals produced by Nova-Seq were identified.

As shown in Table 10, IBS testing is an analysis method that can estimate whether the respective samples are in a familial relationship, and as a result of IBS testing with respect to a human subject, if the average IBS score is 1, the individual is deemed to be the same individual or monozygotic twin, if the average IBS score is 0.5, the individual is deemed to be in a parent-child relationship or full-siblings relationship, and if the average IBS score is 0.25, the individual is deemed to be within a 3-chon relationship, and if the average IBS score is 0.125, the individual is deemed to be within a 4-chon relationship (Anderson C. A. et al., 2010, Nat Protoc).

TABLE 10

| Based on Korean | | |
|---|---|---|
| Kinship System | Relationship | Average IBS score |
| — | Same individual | 1.0 |
| — | Monozygotic twins | 1.0 (0.5) |
| 1-chon | Parent-child | 0.5 (0.25) |
| 2-chon | Full siblings | 0.5 |
| 3-chon | Half siblings | 0.25 |
| 4-chon | First cousins | 0.125 |
| — | Unrelated | 0 |

### 7-1: IBS Test

Using Somalier program (Pedersen et al. 2020), which is a tool capable of rapid evaluation of relatedness from sequencing data, IBS testing was computed for a) variations of the 918-SNP panel according to Example 2-1 described above, or b) variations of the 482-SNP panel according to Example 2-2 described above, from the previously extracted BAM file in Example 6 described above, which is i) data of 40 individuals created by Nova-Seq, or ii) data of 48 individuals created by DNBSEQ-T7 and 2 individuals created by Nova-Seq.

The method of calculating relatedness by IBS testing using Somalier program is described in detail in Pedersen et al. 2020. Somalier program evaluates relatedness between samples by extracting and comparing variant information directly obtained from VCF (variant call format) files or BAM files containing sequence alignments of each sample. Where aligned sequences are evaluated pairwise at a given position, the base aligned at the given position is identified with respect to a given reference base and an alternative base of the variation to be investigated, that is, a base different from the reference base.

Simply put, by pairwise comparison of two targets, if all of the SNP bases at both alleles are identical, IBS score 2 (identity by state 2) is assigned to the SNP, and if only one of the SNP bases is identical, IBS score 1 is assigned to the SNP, and if the SNP bases are all different, IBS score 0 is assigned to the SNP. Next, an average IBS of all SNPs compared pairwise was calculated.

Generally in such IBS testing, the average IBS score of about 1 indicates the same individual or monozygotic twin, the average IBS score of about 0.5 indicates first-d-r which is within 2-chon relationships of parent-child or full-siblings, the average IBS score of about 0.25 indicates second-d-r which is 3-chon relationships, the average IBS score of about 0.125 indicates third-d-r which is 4-chon relationships, and the average IBS score of about 0 indicates no relationship or the unrelated.

Through the Examples described below, it was demonstrated whether it is possible to distinguish individuals who are relatives from those unrelated by using the 918 SNP markers and the 482 SNP markers for kinship identification in Korean according to the present invention, respectively, in an actual Korean population in 1- to 4-chon relationships based on the Korean kinship system, and by calculating average IBS scores between two individuals and dividing the individuals into first-d-r, second-d-r and unrelated groups, or dividing the individuals into first-d-r, second-d-r, third-d-r and unrelated groups.

### 7-2: IBS Testing Using the 918-SNP Panel

7-2-1: Kinship Analysis of Data Generated by Nova-Seq from 40 Individuals

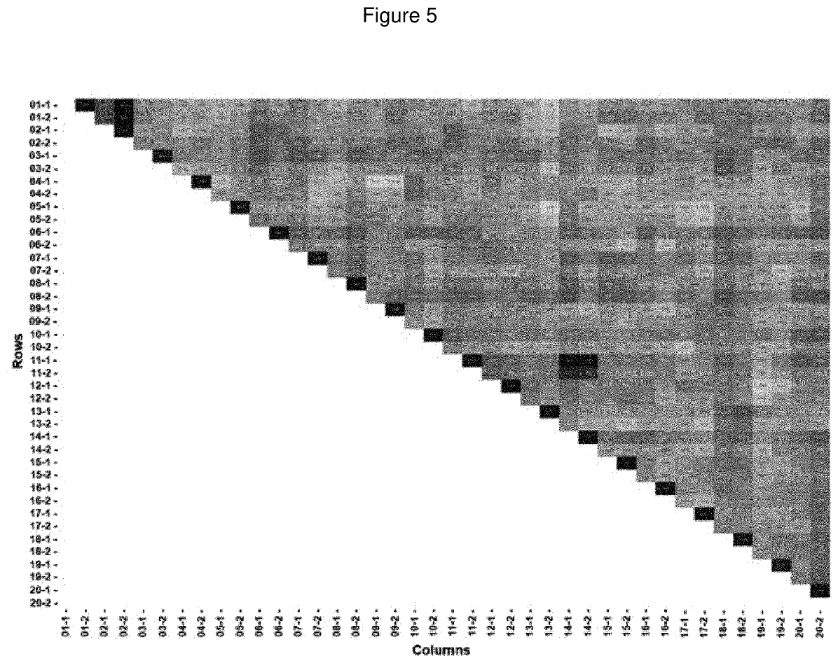

As shown in Example 7-1 described above, IBS test was computed for variations of the 918 SNP markers according to Example 2-1 described above, from previously extracted BAM files using Somalier program (Pedersen et al. 2020), which is a tool that can rapidly evaluate relatedness from sequencing data. Coefficients of relatedness as produced from IBS testing of 40 individuals produced by Nova-Seq using the 918 SNP markers are shown as a comparison matrix in FIG. 5.

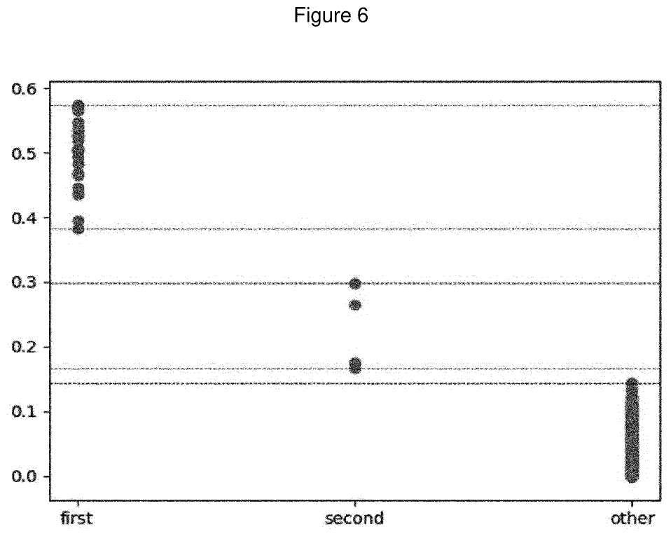

Table 11 below shows average IBS scores between two individuals and their actual familial relationship, and FIG. 6 shows the average IBS score for each group of rirst-d-r, second-d-r and other groups. As a result, the average IBS score of the first-degree relatives was 0.501 (0.382 to 0.574) and the average IBS score of the second-degree relatives was 0.226 (0.166 to 0.299). Also, the average IBS score of the unrelated individuals (other) was −0.014 (−0.193 to 0.144).

From the data indicating that there is no overlap between the buffer region of 0.382 to 0.574 in which the average IBS score of first-d-r is located, and the buffer region of 0.166 to 0.299 in which the average IBS score of second-d-r is located, the present inventors demonstrated that when the above-described 918-SNP panel according to Example 2-1 was applied to Korean families consisting of total 40 individuals, it was possible not only to clearly distinguish first-d-r from those unrelated who are not first-d-r, but also to distinguish between first-d-r and second-d-r.

TABLE 11
IBS scores of 40 individuals using the 918-SNP panel

| Sample 1 | Sample 2 | IBS | Degree |
|---|---|---|---|
| 01-1 | 01-2 | 0.50 | First |
| 01-1 | 02-1 | 0.17 | Second |
| 01-1 | 02-2 | 0.51 | First |
| 01-2 | 02-1 | 0.18 | Second |
| 01-2 | 02-2 | 0.50 | First |
| 02-1 | 02-2 | 0.45 | First |
| 03-1 | 03-2 | 0.38 | First |
| 04-1 | 04-2 | 0.55 | First |
| 05-1 | 05-2 | 0.53 | First |
| 06-1 | 06-2 | 0.53 | First |
| 07-1 | 07-2 | 0.57 | First |
| 08-1 | 08-2 | 0.49 | First |
| 09-1 | 09-2 | 0.44 | First |
| 10-1 | 10-2 | 0.53 | First |
| 11-1 | 11-2 | 0.57 | First |
| 11-1 | 14-1 | 0.52 | First |
| 11-1 | 14-2 | 0.52 | First |
| 11-2 | 14-1 | 0.30 | Second |
| 11-2 | 14-2 | 0.26 | Second |
| 12-1 | 12-2 | 0.50 | First |
| 13-1 | 13-2 | 0.57 | First |
| 14-1 | 14-2 | 0.54 | First |
| 15-1 | 15-2 | 0.48 | First |
| 16-1 | 16-2 | 0.47 | First |
| 17-1 | 17-2 | 0.47 | First |
| 18-1 | 18-2 | 0.57 | First |
| 19-1 | 19-2 | 0.39 | First |
| 20-1 | 20-2 | 0.43 | First |

7-2-2: Kinship Analysis of Data Generated by DNBSEQ-T7 from 48 Individuals and Generated by Nova-Seq from 2 Individuals

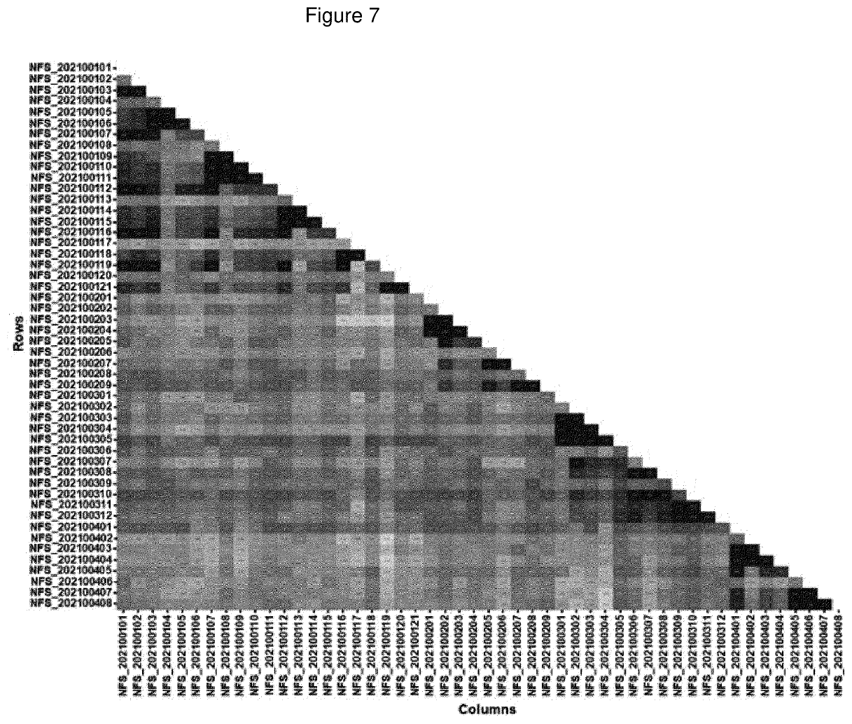

Next, the IBS values were calculated using the data of 48 individuals produced from DNBSEQ-T7 and the data of 2 individuals produced by Nova-Seq. Coefficients of relatedness as produced from IBS testing of 50 individuals using the 918-SNP panel are shown as a comparison matrix in FIG. 7.

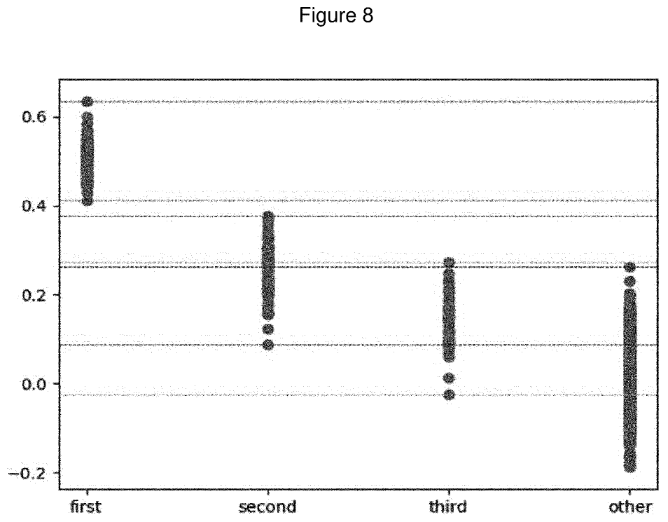

Tables 12 to 14 below show the IBS scores between two individuals in the First-d-r, Second-d-r, and Third-d-r relationships, and FIG. 8 shows the average IBS score for each of First-d-r, Second-d-r, Third-d-r, and Other groups. As a result, the average IBS score of the First-d-r was 0.508 (0.413 to 0.635), the average IBS score of the Second-d-r was 0.253 (0.087 to 0.378), and the average IBS score of the Third-d-r was 0.136 (−0.024 to 0.273). Finally, the average IBS score of the unrelated (other) was 0.025 (−0.187 to 0.263).

From the data indicating that there is no overlap between the buffer region of 0.413 to 0.635 in which the IBS score of first-d-r is located, and the buffer region of 0.087 to 0.378 in which the IBS score of second-d-r is located, the present inventors demonstrated that when the above-described 918-SNP panel according to Example 2-1 was applied to Korean families consisting of total 50 individuals, it was possible not only to clearly distinguish first-d-r from those unrelated who are not first-d-r, but also to distinguish between first-d-r and second-d-r.

TABLE 12
IBS scores of first degree relatives in the results of kinship
analysis by IBS testing in 50 individuals using the 482-SNP panel
Degrees: First degree relatives

| Sample 1 | Sample 2 | IBS |
|---|---|---|
| NFS_202100101 | NFS_202100103 | 0.535 |
| NFS_202100101 | NFS_202100107 | 0.499 |
| NFS_202100101 | NFS_202100112 | 0.52 |
| NFS_202100101 | NFS_202100116 | 0.508 |
| NFS_202100102 | NFS_202100103 | 0.528 |
| NFS_202100102 | NFS_202100107 | 0.47 |
| NFS_202100102 | NFS_202100112 | 0.501 |
| NFS_202100102 | NFS_202100116 | 0.485 |
| NFS_202100103 | NFS_202100105 | 0.492 |
| NFS_202100103 | NFS_202100106 | 0.492 |
| NFS_202100103 | NFS_202100107 | 0.413 |
| NFS_202100103 | NFS_202100112 | 0.635 |
| NFS_202100103 | NFS_202100116 | 0.456 |
| NFS_202100104 | NFS_202100105 | 0.515 |
| NFS_202100104 | NFS_202100106 | 0.475 |
| NFS_202100105 | NFS_202100106 | 0.43 |
| NFS_202100107 | NFS_202100109 | 0.462 |
| NFS_202100107 | NFS_202100110 | 0.496 |
| NFS_202100107 | NFS_202100111 | 0.525 |
| NFS_202100107 | NFS_202100112 | 0.446 |
| NFS_202100107 | NFS_202100116 | 0.558 |
| NFS_202100108 | NFS_202100109 | 0.5 |
| NFS_202100108 | NFS_202100110 | 0.518 |
| NFS_202100108 | NFS_202100111 | 0.523 |
| NFS_202100109 | NFS_202100110 | 0.513 |
| NFS_202100109 | NFS_202100111 | 0.483 |
| NFS_202100110 | NFS_202100111 | 0.585 |
| NFS_202100112 | NFS_202100114 | 0.526 |
| NFS_202100112 | NFS_202100115 | 0.527 |
| NFS_202100112 | NFS_202100116 | 0.449 |
| NFS_202100113 | NFS_202100114 | 0.513 |
| NFS_202100113 | NFS_202100115 | 0.518 |
| NFS_202100114 | NFS_202100115 | 0.549 |
| NFS_202100118 | NFS_202100117 | 0.455 |
| NFS_202100118 | NFS_202100116 | 0.49 |
| NFS_202100119 | NFS_202100121 | 0.446 |
| NFS_202100119 | NFS_202100101 | 0.476 |
| NFS_202100119 | NFS_202100102 | 0.452 |
| NFS_202100119 | NFS_202100103 | 0.495 |
| NFS_202100119 | NFS_202100107 | 0.453 |
| NFS_202100119 | NFS_202100112 | 0.489 |
| NFS_202100119 | NFS_202100116 | 0.485 |
| NFS_202100121 | NFS_202100120 | 0.531 |
| NFS_202100203 | NFS_202100201 | 0.475 |
| NFS_202100203 | NFS_202100202 | 0.47 |
| NFS_202100204 | NFS_202100203 | 0.527 |
| NFS_202100204 | NFS_202100201 | 0.529 |
| NFS_202100204 | NFS_202100202 | 0.508 |
| NFS_202100205 | NFS_202100207 | 0.512 |
| NFS_202100205 | NFS_202100202 | 0.527 |
| NFS_202100206 | NFS_202100207 | 0.46 |
| NFS_202100207 | NFS_202100209 | 0.527 |
| NFS_202100208 | NFS_202100209 | 0.515 |
| NFS_202100301 | NFS_202100304 | 0.513 |
| NFS_202100301 | NFS_202100305 | 0.549 |
| NFS_202100301 | NFS_202100303 | 0.527 |
| NFS_202100302 | NFS_202100303 | 0.511 |
| NFS_202100302 | NFS_202100307 | 0.506 |
| NFS_202100304 | NFS_202100305 | 0.541 |
| NFS_202100304 | NFS_202100302 | 0.511 |
| NFS_202100304 | NFS_202100303 | 0.527 |
| NFS_202100305 | NFS_202100302 | 0.526 |
| NFS_202100305 | NFS_202100303 | 0.527 |
| NFS_202100306 | NFS_202100310 | 0.552 |
| NFS_202100308 | NFS_202100306 | 0.519 |
| NFS_202100308 | NFS_202100310 | 0.548 |
| NFS_202100308 | NFS_202100307 | 0.51 |
| NFS_202100309 | NFS_202100312 | 0.534 |
| NFS_202100310 | NFS_202100307 | 0.506 |
| NFS_202100311 | NFS_202100309 | 0.526 |
| NFS_202100311 | NFS_202100312 | 0.569 |
| NFS_202100311 | NFS_202100310 | 0.563 |
| NFS_202100312 | NFS_202100310 | 0.54 |
| NFS_202100401 | NFS_202100403 | 0.521 |
| NFS_202100401 | NFS_202100404 | 0.531 |
| NFS_202100402 | NFS_202100403 | 0.478 |
| NFS_202100402 | NFS_202100404 | 0.467 |
| NFS_202100403 | NFS_202100404 | 0.442 |
| NFS_202100405 | NFS_202100401 | 0.599 |
| NFS_202100405 | NFS_202100407 | 0.522 |
| NFS_202100405 | NFS_202100408 | 0.519 |
| NFS_202100406 | NFS_202100407 | 0.482 |
| NFS_202100406 | NFS_202100408 | 0.52 |
| NFS_202100407 | NFS_202100408 | 0.521 |

TABLE 13
IBS scores of second degree relatives in the results of kinship
analysis by IBS testing in 50 individuals using the 482-SNP panel
Degree: Second degree relatives

| Sample 1 | Sample 2 | IBS |
|---|---|---|
| NFS_202100101 | NFS_202100105 | 0.206 |
| NFS_202100101 | NFS_202100106 | 0.23 |
| NFS_202100101 | NFS_202100109 | 0.249 |
| NFS_202100101 | NFS_202100110 | 0.326 |
| NFS_202100101 | NFS_202100111 | 0.302 |
| NFS_202100101 | NFS_202100114 | 0.279 |
| NFS_202100101 | NFS_202100115 | 0.309 |
| NFS_202100102 | NFS_202100105 | 0.289 |
| NFS_202100102 | NFS_202100106 | 0.271 |
| NFS_202100102 | NFS_202100109 | 0.208 |
| NFS_202100102 | NFS_202100110 | 0.216 |
| NFS_202100102 | NFS_202100111 | 0.245 |
| NFS_202100102 | NFS_202100114 | 0.183 |
| NFS_202100102 | NFS_202100115 | 0.232 |
| NFS_202100103 | NFS_202100109 | 0.122 |
| NFS_202100103 | NFS_202100110 | 0.281 |
| NFS_202100103 | NFS_202100111 | 0.259 |
| NFS_202100103 | NFS_202100114 | 0.303 |
| NFS_202100103 | NFS_202100115 | 0.367 |
| NFS_202100105 | NFS_202100107 | 0.154 |
| NFS_202100105 | NFS_202100112 | 0.33 |
| NFS_202100105 | NFS_202100116 | 0.175 |
| NFS_202100106 | NFS_202100107 | 0.177 |
| NFS_202100106 | NFS_202100112 | 0.354 |
| NFS_202100106 | NFS_202100116 | 0.216 |
| NFS_202100107 | NFS_202100114 | 0.217 |
| NFS_202100107 | NFS_202100115 | 0.265 |
| NFS_202100109 | NFS_202100112 | 0.232 |
| NFS_202100109 | NFS_202100116 | 0.272 |
| NFS_202100110 | NFS_202100112 | 0.237 |
| NFS_202100110 | NFS_202100116 | 0.309 |
| NFS_202100111 | NFS_202100112 | 0.254 |
| NFS_202100111 | NFS_202100116 | 0.333 |
| NFS_202100114 | NFS_202100116 | 0.205 |
| NFS_202100115 | NFS_202100116 | 0.233 |
| NFS_202100118 | NFS_202100119 | 0.155 |
| NFS_202100118 | NFS_202100101 | 0.194 |
| NFS_202100118 | NFS_202100102 | 0.306 |
| NFS_202100118 | NFS_202100103 | 0.225 |
| NFS_202100118 | NFS_202100107 | 0.272 |
| NFS_202100118 | NFS_202100112 | 0.161 |
| NFS_202100119 | NFS_202100105 | 0.087 |
| NFS_202100119 | NFS_202100106 | 0.21 |
| NFS_202100119 | NFS_202100109 | 0.168 |
| NFS_202100119 | NFS_202100110 | 0.2 |
| NFS_202100119 | NFS_202100111 | 0.237 |
| NFS_202100119 | NFS_202100114 | 0.158 |
| NFS_202100119 | NFS_202100115 | 0.212 |
| NFS_202100121 | NFS_202100101 | 0.292 |
| NFS_202100121 | NFS_202100102 | 0.223 |
| NFS_202100121 | NFS_202100103 | 0.288 |
| NFS_202100121 | NFS_202100107 | 0.223 |
| NFS_202100121 | NFS_202100112 | 0.286 |
| NFS_202100121 | NFS_202100116 | 0.238 |
| NFS_202100203 | NFS_202100205 | 0.214 |
| NFS_202100204 | NFS_202100205 | 0.272 |
| NFS_202100205 | NFS_202100209 | 0.372 |
| NFS_202100206 | NFS_202100209 | 0.199 |
| NFS_202100207 | NFS_202100202 | 0.279 |
| NFS_202100303 | NFS_202100307 | 0.297 |
| NFS_202100304 | NFS_202100307 | 0.256 |
| NFS_202100305 | NFS_202100307 | 0.301 |
| NFS_202100308 | NFS_202100312 | 0.343 |
| NFS_202100308 | NFS_202100302 | 0.27 |
| NFS_202100310 | NFS_202100302 | 0.304 |
| NFS_202100311 | NFS_202100308 | 0.32 |
| NFS_202100311 | NFS_202100306 | 0.304 |
| NFS_202100311 | NFS_202100307 | 0.251 |
| NFS_202100312 | NFS_202100306 | 0.378 |
| NFS_202100312 | NFS_202100307 | 0.211 |
| NFS_202100401 | NFS_202100407 | 0.331 |
| NFS_202100401 | NFS_202100408 | 0.324 |
| NFS_202100405 | NFS_202100403 | 0.289 |
| NFS_202100405 | NFS_202100404 | 0.267 |

TABLE 14
IBS scores of third degree relatives in the results of kinship
analysis by IBS testing in 50 individuals using the 918-SNP panel
Degree: Third degree relatives

| Sample 1 | Sample 2 | IBS | Sample 1 | Sample 2 | IBS |
|---|---|---|---|---|---|
| NFS_202100105 | NFS_202100109 | 0.014 | NFS_202100118 | NFS_202100115 | 0.103 |
| NFS_202100105 | NFS_202100110 | 0.093 | NFS_202100121 | NFS_202100105 | 0.072 |
| NFS_202100105 | NFS_202100111 | 0.083 | NFS_202100121 | NFS_202100106 | 0.108 |
| NFS_202100105 | NFS_202100114 | 0.15 | NFS_202100121 | NFS_202100109 | 0.143 |
| NFS_202100105 | NFS_202100115 | 0.175 | NFS_202100121 | NFS_202100110 | 0.131 |
| NFS_202100106 | NFS_202100109 | −0.024 | NFS_202100121 | NFS_202100111 | 0.137 |
| NFS_202100106 | NFS_202100110 | 0.144 | NFS_202100121 | NFS_202100114 | 0.154 |
| NFS_202100106 | NFS_202100111 | 0.12 | NFS_202100121 | NFS_202100115 | 0.169 |
| NFS_202100106 | NFS_202100114 | 0.143 | NFS_202100203 | NFS_202100207 | 0.119 |
| NFS_202100106 | NFS_202100115 | 0.164 | NFS_202100204 | NFS_202100207 | 0.112 |
| NFS_202100109 | NFS_202100114 | 0.103 | NFS_202100209 | NFS_202100202 | 0.232 |
| NFS_202100109 | NFS_202100115 | 0.114 | NFS_202100304 | NFS_202100308 | 0.151 |
| NFS_202100110 | NFS_202100114 | 0.101 | NFS_202100304 | NFS_202100310 | 0.162 |
| NFS_202100110 | NFS_202100115 | 0.164 | NFS_202100305 | NFS_202100310 | 0.273 |
| NFS_202100111 | NFS_202100114 | 0.173 | NFS_202100308 | NFS_202100305 | 0.247 |
| NFS_202100111 | NFS_202100115 | 0.208 | NFS_202100308 | NFS_202100303 | 0.225 |
| NFS_202100118 | NFS_202100121 | 0.077 | NFS_202100310 | NFS_202100303 | 0.215 |
| NFS_202100118 | NFS_202100105 | 0.069 | NFS_202100311 | NFS_202100302 | 0.167 |
| NFS_202100118 | NFS_202100106 | 0.086 | NFS_202100312 | NFS_202100302 | 0.149 |
| NFS_202100118 | NFS_202100109 | 0.153 | NFS_202100403 | NFS_202100407 | 0.184 |
| NFS_202100118 | NFS_202100110 | 0.18 | NFS_202100403 | NFS_202100408 | 0.192 |
| NFS_202100118 | NFS_202100111 | 0.206 | NFS_202100404 | NFS_202100407 | 0.09 |
| NFS_202100118 | NFS_202100114 | 0.06 | NFS_202100404 | NFS_202100408 | 0.095 |
| NFS_202100118 | NFS_202100114 | 0.06 | NFS_202100118 | NFS_202100115 | 0.103 |

### 7-3: IBS Testing Using the 482-SNP Panel

7-3-1: Kinship Analysis of Data Generated by Nova-Seq from 40 Individuals

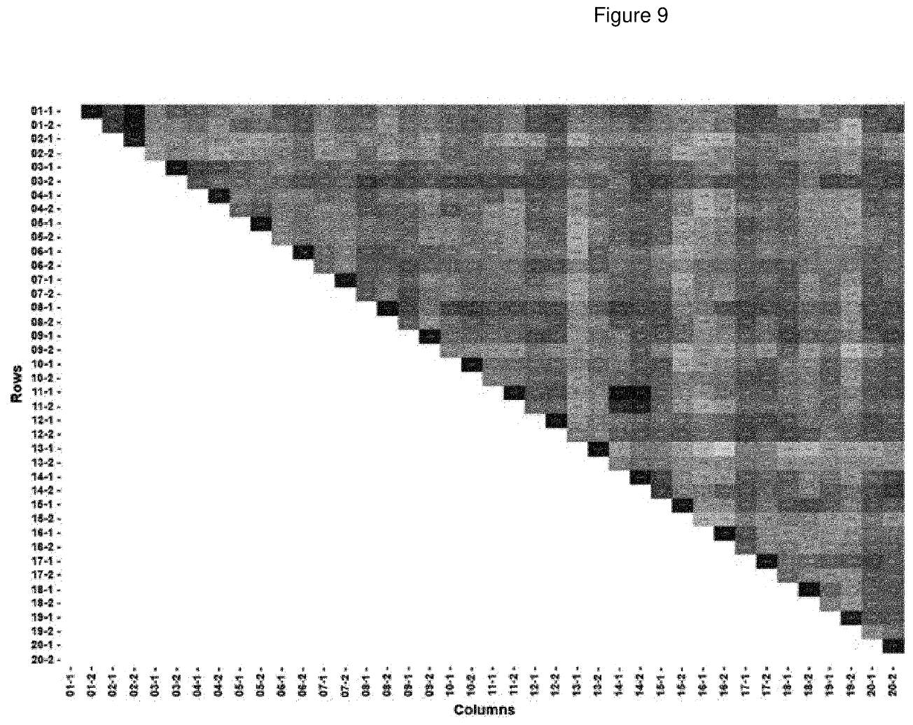

The IBS test was computed for variations of the 482-SNP panel according to Example 2-2 described above, from the BAM files of 40 individuals produced with Nova-Seq using the Somalier program. IBS scores as produced from IBS testing of 40 individuals using the 482-SNP panel are shown as a comparison matrix in FIG. 9.

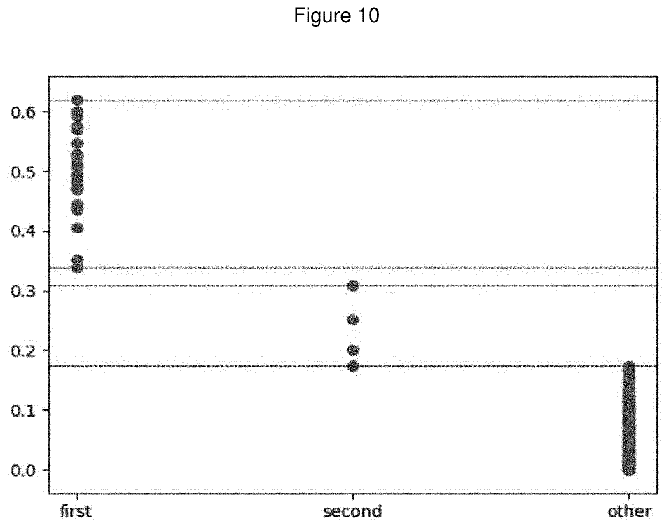

Table 15 below shows average IBS scores between two individuals and their actual familial relationship, and FIG. 10 shows the average IBS score for each group of first-d-r, second-d-r and other groups. As a result, the average IBS score of first-d-r was 0.496 (0.340 to 0.620), and the average IBS score of second-d-r was 0.233 (0.175 to 0.309). In addition, the average IBS score of the unrelated (other) was −0.02 (−0.277 to 0.175).

From the data indicating that there is no overlap between the buffer region of 0.340 to 0.620 in which the coefficient of relatedness of first-d-r is located, and the buffer region of 0.175 to 0.309 in which the coefficient of relatedness of second-d-r is located, the present inventors were able to confirm that when the above-described 482-SNP panel according to Example 2-2 was applied to Korean families consisting of total 40 individuals, it was possible to clearly distinguish first-d-r from the unrelated.

TABLE 15
IBS scores of 40 individuals using 482 SNP markers

| Sample 1 | Sample 2 | IBS | Degree |
|---|---|---|---|
| 01-1 | 01-2 | 0.58 | First |
| 01-1 | 02-1 | 0.17 | Second |
| 01-1 | 02-2 | 0.52 | First |
| 01-2 | 02-1 | 0.2 | Second |
| 01-2 | 02-2 | 0.55 | First |
| 02-1 | 02-2 | 0.35 | First |
| 03-1 | 03-2 | 0.44 | First |
| 04-1 | 04-2 | 0.6 | First |
| 05-1 | 05-2 | 0.53 | First |
| 06-1 | 06-2 | 0.53 | First |
| 07-1 | 07-2 | 0.62 | First |
| 08-1 | 08-2 | 0.47 | First |
| 09-1 | 09-2 | 0.45 | First |
| 10-1 | 10-2 | 0.49 | First |
| 11-1 | 11-2 | 0.59 | First |
| 11-1 | 14-1 | 0.47 | First |
| 11-1 | 14-2 | 0.51 | First |
| 11-2 | 14-1 | 0.25 | Second |
| 11-2 | 14-2 | 0.31 | Second |
| 12-1 | 12-2 | 0.47 | First |
| 13-1 | 13-2 | 0.51 | First |
| 14-1 | 14-2 | 0.57 | First |
| 15-1 | 15-2 | 0.44 | First |
| 16-1 | 16-2 | 0.48 | First |
| 17-1 | 17-2 | 0.48 | First |
| 18-1 | 18-2 | 0.5 | First |
| 19-1 | 19-2 | 0.34 | First |
| 20-1 | 20-2 | 0.41 | First |

7-3-2: Kinship Analysis of Data Generated by DNBSEQ-T7 from 48 Individuals and Generated by Nova-Seq from 2 Individuals

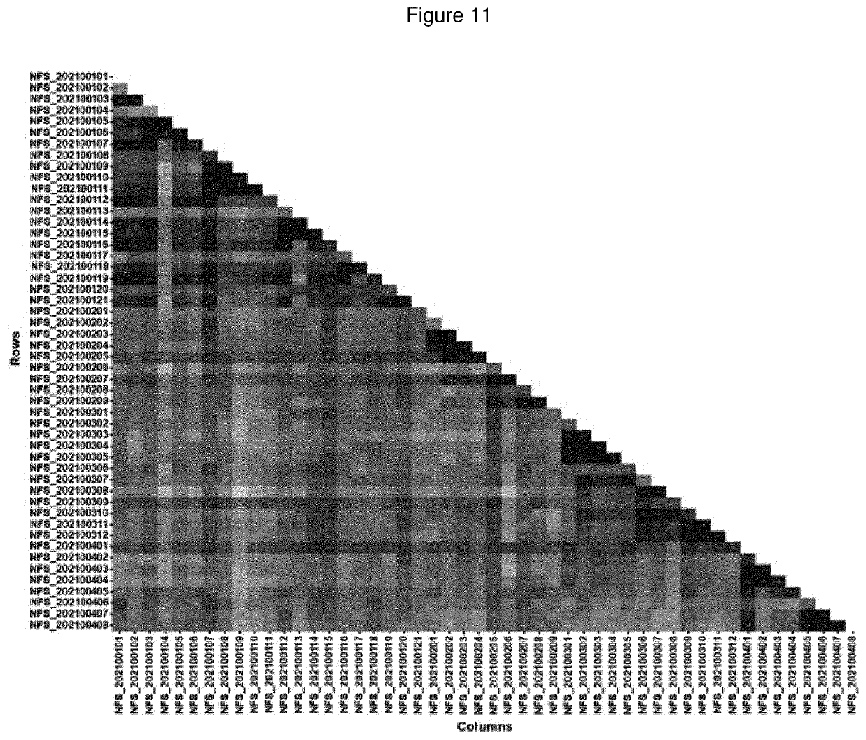

Next, using the data of 48 individuals generated by DNBSEQ-T7 and the data of 2 individuals generated by Nova-Seq, IBS scores by IBS testing were calculated. IBS scores as produced from IBS testing of 50 individuals using the 482 SNP markers are shown as a comparison matrix in FIG. 11.

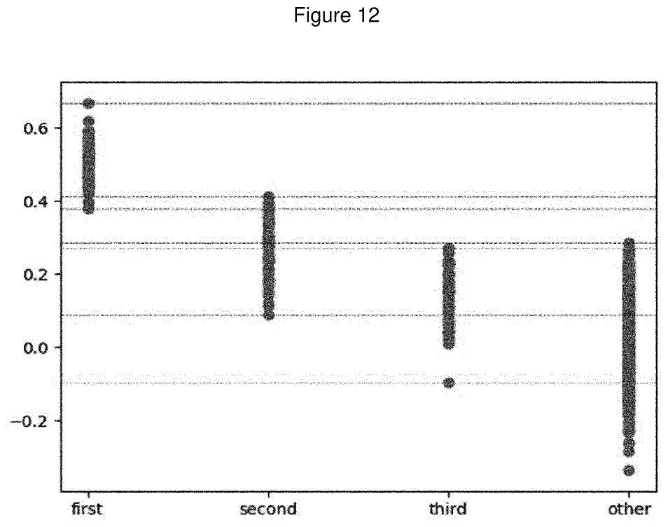

Tables 16 to 19 below show the IBS scores between two individuals in the First-d-r, Second-d-r, and Third-d-r relationships, respectively, and FIG. 12 shows the average IBS score for each of First-d-r, Second-d-r, Third-d-r, and Other groups. As a result, the average IBS score of the First-d-r was 0.509 (0.378 to 0.667), the average IBS score of the Second-d-r was 0.280 (0.087 to 0.414), and the average IBS score of the Third-d-r was 0.139 (−0.096 to 0.270). Finally, the average IBS score between the unrelated (other) was 0.020 (−0.335 to 0.285).

Although there is a slight overlap between the buffer region of 0.378 to 0.667 in which the IBS scores of first-d-r are located and the buffer region of 0.087 to 0.414 in which the IBS scores of second-d-r are located, the present inventors have confirmed that the 482-SNP panel according to Example 2-2 when used on families of 50 Korean individuals, is useful to obtain information capable of distinguishing, with respect to a subject, individuals who are in first-d-r relationships, individuals who are possibly in first-d-r relationships, or individuals who are highly likely in first-d-r relationships, from the unrelated, that is, individuals who are not in first-d-r relationships, individuals who are not likely in first-d-r relationships, or individuals who are less likely to be in first-d-r relationships.

TABLE 16
IBS score of first degree relatives in kinship analysis results
by IBS testing in 50 individuals using the 482-SNP panel
Degree: First degree relatives

| Sample 1 | Sample 2 | IBS |
|---|---|---|
| NFS_202100101 | NFS_202100103 | 0.494 |
| NFS_202100101 | NFS_202100107 | 0.518 |
| NFS_202100101 | NFS_202100112 | 0.541 |
| NFS_202100101 | NFS_202100116 | 0.491 |
| NFS_202100102 | NFS_202100103 | 0.514 |
| NFS_202100102 | NFS_202100107 | 0.536 |
| NFS_202100102 | NFS_202100112 | 0.494 |
| NFS_202100102 | NFS_202100116 | 0.426 |
| NFS_202100103 | NFS_202100105 | 0.532 |
| NFS_202100103 | NFS_202100106 | 0.459 |
| NFS_202100103 | NFS_202100107 | 0.552 |
| NFS_202100103 | NFS_202100112 | 0.667 |
| NFS_202100103 | NFS_202100116 | 0.421 |
| NFS_202100104 | NFS_202100105 | 0.457 |
| NFS_202100104 | NFS_202100106 | 0.465 |
| NFS_202100105 | NFS_202100106 | 0.509 |
| NFS_202100107 | NFS_202100109 | 0.503 |
| NFS_202100107 | NFS_202100110 | 0.525 |
| NFS_202100107 | NFS_202100111 | 0.561 |
| NFS_202100107 | NFS_202100112 | 0.476 |
| NFS_202100107 | NFS_202100116 | 0.592 |
| NFS_202100108 | NFS_202100109 | 0.497 |
| NFS_202100108 | NFS_202100110 | 0.508 |
| NFS_202100108 | NFS_202100111 | 0.537 |
| NFS_202100109 | NFS_202100110 | 0.398 |
| NFS_202100109 | NFS_202100111 | 0.443 |
| NFS_202100110 | NFS_202100111 | 0.513 |
| NFS_202100112 | NFS_202100114 | 0.551 |
| NFS_202100112 | NFS_202100115 | 0.522 |
| NFS_202100112 | NFS_202100116 | 0.44 |
| NFS_202100113 | NFS_202100114 | 0.516 |
| NFS_202100113 | NFS_202100115 | 0.513 |
| NFS_202100114 | NFS_202100115 | 0.571 |
| NFS_202100118 | NFS_202100117 | 0.513 |
| NFS_202100118 | NFS_202100116 | 0.506 |
| NFS_202100119 | NFS_202100121 | 0.495 |
| NFS_202100119 | NFS_202100101 | 0.49 |
| NFS_202100119 | NFS_202100102 | 0.519 |
| NFS_202100119 | NFS_202100103 | 0.556 |
| NFS_202100119 | NFS_202100107 | 0.563 |
| NFS_202100119 | NFS_202100112 | 0.572 |
| NFS_202100119 | NFS_202100116 | 0.526 |
| NFS_202100121 | NFS_202100120 | 0.523 |
| NFS_202100203 | NFS_202100201 | 0.516 |
| NFS_202100203 | NFS_202100202 | 0.495 |
| NFS_202100204 | NFS_202100203 | 0.493 |
| NFS_202100204 | NFS_202100201 | 0.46 |
| NFS_202100204 | NFS_202100202 | 0.522 |
| NFS_202100205 | NFS_202100207 | 0.573 |
| NFS_202100205 | NFS_202100202 | 0.526 |
| NFS_202100206 | NFS_202100207 | 0.449 |
| NFS_202100207 | NFS_202100209 | 0.509 |
| NFS_202100208 | NFS_202100209 | 0.53 |
| NFS_202100301 | NFS_202100304 | 0.499 |
| NFS_202100301 | NFS_202100305 | 0.525 |
| NFS_202100301 | NFS_202100303 | 0.461 |
| NFS_202100302 | NFS_202100303 | 0.496 |
| NFS_202100302 | NFS_202100307 | 0.592 |
| NFS_202100304 | NFS_202100305 | 0.54 |
| NFS_202100304 | NFS_202100302 | 0.535 |
| NFS_202100304 | NFS_202100303 | 0.464 |
| NFS_202100305 | NFS_202100302 | 0.523 |
| NFS_202100305 | NFS_202100303 | 0.472 |
| NFS_202100306 | NFS_202100310 | 0.482 |
| NFS_202100308 | NFS_202100306 | 0.462 |
| NFS_202100308 | NFS_202100310 | 0.437 |
| NFS_202100308 | NFS_202100307 | 0.437 |
| NFS_202100309 | NFS_202100312 | 0.573 |
| NFS_202100310 | NFS_202100307 | 0.526 |
| NFS_202100311 | NFS_202100309 | 0.59 |
| NFS_202100311 | NFS_202100312 | 0.554 |
| NFS_202100311 | NFS_202100310 | 0.496 |
| NFS_202100312 | NFS_202100310 | 0.538 |
| NFS_202100401 | NFS_202100403 | 0.479 |
| NFS_202100401 | NFS_202100404 | 0.551 |
| NFS_202100402 | NFS_202100403 | 0.545 |
| NFS_202100402 | NFS_202100404 | 0.525 |
| NFS_202100403 | NFS_202100404 | 0.378 |
| NFS_202100405 | NFS_202100401 | 0.619 |
| NFS_202100405 | NFS_202100407 | 0.494 |
| NFS_202100405 | NFS_202100408 | 0.495 |
| NFS_202100406 | NFS_202100407 | 0.469 |
| NFS_202100406 | NFS_202100408 | 0.443 |
| NFS_202100407 | NFS_202100408 | 0.453 |

TABLE 17
IBS scores of second degree relatives in the results of kinship
analysis by IBS testing in 50 individuals using the 482-SNP panel
Degree: Second degree relatives

| Sample 1 | Sample 2 | IBS | Sample 1 | Sample 2 | IBS |
|---|---|---|---|---|---|
| NFS_202100204 | NFS_202100205 | 0.385 | NFS_202100207 | NFS_202100202 | 0.292 |
| NFS_202100203 | NFS_202100205 | 0.356 | NFS_202100101 | NFS_202100105 | 0.278 |
| NFS_202100205 | NFS_202100209 | 0.33 | NFS_202100101 | NFS_202100106 | 0.211 |
| NFS_202100405 | NFS_202100403 | 0.237 | NFS_202100101 | NFS_202100109 | 0.148 |
| NFS_202100405 | NFS_202100404 | 0.284 | NFS_202100101 | NFS_202100110 | 0.156 |
| NFS_202100118 | NFS_202100119 | 0.302 | NFS_202100101 | NFS_202100111 | 0.202 |
| NFS_202100118 | NFS_202100101 | 0.216 | NFS_202100101 | NFS_202100114 | 0.313 |
| NFS_202100118 | NFS_202100102 | 0.36 | NFS_202100101 | NFS_202100115 | 0.333 |
| NFS_202100118 | NFS_202100103 | 0.274 | NFS_202100102 | NFS_202100105 | 0.347 |
| NFS_202100118 | NFS_202100107 | 0.393 | NFS_202100102 | NFS_202100106 | 0.183 |
| NFS_202100118 | NFS_202100112 | 0.291 | NFS_202100102 | NFS_202100109 | 0.113 |
| NFS_202100311 | NFS_202100308 | 0.167 | NFS_202100102 | NFS_202100110 | 0.178 |
| NFS_202100311 | NFS_202100306 | 0.342 | NFS_202100102 | NFS_202100111 | 0.274 |
| NFS_202100311 | NFS_202100307 | 0.256 | NFS_202100102 | NFS_202100114 | 0.273 |
| NFS_202100304 | NFS_202100307 | 0.3 | NFS_202100102 | NFS_202100115 | 0.239 |
| NFS_202100308 | NFS_202100312 | 0.248 | NFS_202100103 | NFS_202100109 | 0.144 |
| NFS_202100308 | NFS_202100302 | 0.216 | NFS_202100103 | NFS_202100110 | 0.242 |
| NFS_202100312 | NFS_202100306 | 0.372 | NFS_202100103 | NFS_202100111 | 0.295 |
| NFS_202100312 | NFS_202100307 | 0.266 | NFS_202100103 | NFS_202100114 | 0.397 |
| NFS_202100401 | NFS_202100407 | 0.294 | NFS_202100103 | NFS_202100115 | 0.355 |
| NFS_202100401 | NFS_202100408 | 0.341 | NFS_202100105 | NFS_202100107 | 0.394 |
| NFS_202100305 | NFS_202100307 | 0.414 | NFS_202100105 | NFS_202100112 | 0.384 |
| NFS_202100310 | NFS_202100302 | 0.301 | NFS_202100105 | NFS_202100116 | 0.284 |
| NFS_202100119 | NFS_202100105 | 0.356 | NFS_202100106 | NFS_202100107 | 0.297 |
| NFS_202100119 | NFS_202100106 | 0.305 | NFS_202100106 | NFS_202100112 | 0.327 |
| NFS_202100119 | NFS_202100109 | 0.169 | NFS_202100106 | NFS_202100116 | 0.231 |
| NFS_202100119 | NFS_202100110 | 0.215 | NFS_202100107 | NFS_202100114 | 0.355 |
| NFS_202100119 | NFS_202100111 | 0.351 | NFS_202100107 | NFS_202100115 | 0.376 |
| NFS_202100119 | NFS_202100114 | 0.303 | NFS_202100109 | NFS_202100112 | 0.087 |
| NFS_202100119 | NFS_202100115 | 0.363 | NFS_202100109 | NFS_202100116 | 0.23 |
| NFS_202100121 | NFS_202100101 | 0.232 | NFS_202100110 | NFS_202100112 | 0.121 |
| NFS_202100121 | NFS_202100102 | 0.29 | NFS_202100110 | NFS_202100116 | 0.245 |
| NFS_202100121 | NFS_202100103 | 0.334 | NFS_202100111 | NFS_202100112 | 0.177 |
| NFS_202100121 | NFS_202100107 | 0.376 | NFS_202100111 | NFS_202100116 | 0.292 |
| NFS_202100121 | NFS_202100112 | 0.343 | NFS_202100114 | NFS_202100116 | 0.308 |
| NFS_202100121 | NFS_202100116 | 0.273 | NFS_202100115 | NFS_202100116 | 0.286 |
| NFS_202100206 | NFS_202100209 | 0.185 | NFS_202100303 | NFS_202100307 | 0.296 |

TABLE 18
IBS scores of third degree relatives in the results of kinship
analysis by IBS testing in 50 individuals using the 482-SNP panel
Degree: Third degree relatives

| Sample 1 | Sample 2 | IBS | Sample 1 | Sample 2 | IBS |
|---|---|---|---|---|---|
| NFS_202100204 | NFS_202100207 | 0.199 | NFS_202100121 | NFS_202100106 | 0.076 |
| NFS_202100203 | NFS_202100207 | 0.257 | NFS_202100121 | NFS_202100109 | 0.044 |
| NFS_202100118 | NFS_202100121 | 0.227 | NFS_202100121 | NFS_202100110 | 0.03 |
| NFS_202100118 | NFS_202100105 | 0.169 | NFS_202100121 | NFS_202100111 | 0.136 |
| NFS_202100118 | NFS_202100106 | 0.11 | NFS_202100121 | NFS_202100114 | 0.226 |
| NFS_202100118 | NFS_202100109 | 0.153 | NFS_202100121 | NFS_202100115 | 0.269 |
| NFS_202100118 | NFS_202100110 | 0.154 | NFS_202100209 | NFS_202100202 | 0.092 |
| NFS_202100118 | NFS_202100111 | 0.202 | NFS_202100105 | NFS_202100109 | 0.044 |
| NFS_202100118 | NFS_202100114 | 0.236 | NFS_202100105 | NFS_202100110 | 0.149 |
| NFS_202100118 | NFS_202100115 | 0.226 | NFS_202100105 | NFS_202100111 | 0.2 |
| NFS_202100311 | NFS_202100302 | 0.169 | NFS_202100105 | NFS_202100114 | 0.27 |
| NFS_202100304 | NFS_202100308 | 0.104 | NFS_202100105 | NFS_202100115 | 0.231 |
| NFS_202100304 | NFS_202100310 | 0.19 | NFS_202100106 | NFS_202100109 | −0.096 |
| NFS_202100308 | NFS_202100305 | 0.114 | NFS_202100106 | NFS_202100110 | 0.096 |
| NFS_202100308 | NFS_202100303 | 0.063 | NFS_202100106 | NFS_202100111 | 0.124 |
| NFS_202100312 | NFS_202100302 | 0.155 | NFS_202100106 | NFS_202100114 | 0.179 |
| NFS_202100403 | NFS_202100407 | 0.054 | NFS_202100106 | NFS_202100115 | 0.205 |
| NFS_202100403 | NFS_202100408 | 0.039 | NFS_202100109 | NFS_202100114 | 0.043 |
| NFS_202100404 | NFS_202100407 | 0.01 | NFS_202100109 | NFS_202100115 | 0.019 |
| NFS_202100404 | NFS_202100408 | 0.062 | NFS_202100110 | NFS_202100114 | 0.125 |
| NFS_202100305 | NFS_202100310 | 0.179 | NFS_202100110 | NFS_202100115 | 0.071 |
| NFS_202100310 | NFS_202100303 | 0.221 | NFS_202100111 | NFS_202100114 | 0.151 |
| NFS_202100121 | NFS_202100105 | 0.232 | NFS_202100111 | NFS_202100115 | 0.163 |
| NFS_202100204 | NFS_202100207 | 0.199 | NFS_202100121 | NFS_202100106 | 0.076 |

### Conclusion

It was demonstrated that when using the 918-SNP panel and the 482-SNP panel according to the present invention discovered from SNPs of unrelated 88 Korean individuals, in a group of Korean individuals who are in 1- to 4-chon relationships based on the Korean kinship system, by calculating average IBS scores from IBS testing of making pairwise comparison of each base at each SNP between two individuals, individuals who are first-d-r with respect to a subject and individuals who are not first-d-r can be clearly distinguished from the calculated average IBS scores.

As summarized in Table 19 below, when using the 918-SNP panel according to Example 2-1 of the present invention on the WGS data of 40 individuals produced by Nova-Seq, which is a commercially available sequencing platform, the average IBS score of first-d-r was found to be 0.501 and the average IBS score of second-d-r was found to be 0.226. By using a different marker set, that is, by varying the panel, when the 482-SNP panel according to Example 2-2 was used, the average IBS score of first-d-r was 0.496 and the average IBS score of second-d-r was 0.233.

TABLE 19

| Data generated by Nova-Seq | | |
|---|---|---|
| from 40 individuals | 918-SNP panel | 482-SNP panel |
| Average of coefficients of | | |
| relatedness by IBS testing of | 0.501 | 0.496 |
| first-degree relatives | (0.382 to 0.574) | (0.340 to 0.620) |
| (Buffer region) | | |
| Average of coefficients of | 0.226 | 0.233 |
| relatedness by IBS testing of | (0.166 to 0.299) | (0.175 to 0.309) |
| second-degree relatives | | |
| (Buffer region) | | |
| Average of coefficients of | −0.014 | −0.02 |
| relatedness by IBS testing | (−0.193 to 0.144) | (−0.277 to 0.175) |
| of the unrelated | | |
| (Buffer region) | | |

Also as summarized in Table 20 below, when the 918-SNP panel according to Example 2-1 was used on the WGS data of 48 individuals produced by DNBSEQ-T7 and the WGS data of 2 individuals produced by Nova-Seq, which are commercially available sequencing platforms, the average IBS score of first-d-r was 0.508 and the average IBS score of second-d-r was 0.253. By using a different marker set, that is, by varying the panel, when the 482-SNP panel according to Example 2-2 was used, the average IBS score of first-d-r was 0.509 and the average IBS score of second-d-r was 0.280.

TABLE 20

| Data generated by DNBSEQ-T7 | | |
|---|---|---|
| from 48 individuals and | | |
| generated by Nova-Seq from | | |
| 2 individuals | 918-SNP panel | 482-SNP panel |
| Average of coefficients of | 0.508 | 0.509 |
| relatedness by IBS testing of | (0.413 to 0.635) | (0.378 to 0.667) |
| first-degree relatives | | |
| (Buffer region) | | |
| Average of coefficients of | 0.253 | 0.280 |
| relatedness by IBS testing of | (0.087 to 0.378) | (0.087 to 0.414) |
| second-degree relatives | | |
| (Buffer region) | | |
| Average of coefficients of | 0.136 | 0.139 |
| relatedness by IBS testing of | (−0.024 to 0.273) | (−0.096 to 0.270) |
| third-degree relatives | | |
| (Buffer region) | | |
| Average of coefficients of | 0.025 | 0.020 |
| relatedness by IBS testing of | (−0.187 to 0.263) | (−0.335 to 0.285) |
| the unrelated (Buffer region) | | |

More importantly, when using the 918 SNP markers according to the present invention, even when a smaller number of SNPs is checked compared to the prior art methods, and even when no parent information is available, since the buffer regions in which the average IBS score of two individuals in a first-degree relationship is located and the buffer region in which the average IBS score of two individuals in a second-degree relationship is located are clearly separated from each other, that is, the two buffer regions do not overlap but are distinguished from each other, it is possible to clearly distinguish, with respect to a subject, individuals who are in a first-d-r relationship as one of parent, child, brother, sister, and sibling, from individuals who are not in any first-d-r relationship.

Also, when using the 482 SNP markers according to the present invention, the buffer region in which an average IBS score of two individuals in a first-d-r relationship is located partially overlaps with the buffer region in which an average IBS score of two individuals in a second-d-r relationship is located. Therefore, the use of the 482 SNP markers may be slightly limited in distinguishing individuals in first-d-r relationships with a subject, from individuals who are in second-d-r relationships with the subject, but can be still useful in distinguishing individuals who are in first-d-r relationships with the subject from those who are not in any first-d-r relationships. In the field of forensic science, any evidence indicating that the possibility of two individuals being in a familial relationship cannot be excluded holds significance. Since the 482 SNP markers according to the present invention makes it possible to distinguish individuals in a first-d-r relationship from the unrelated, individuals possibly in a first-d-r relationship from the unrelated, or individuals highly likely in a first-d-r relationship from the unrelated by simple pairwise comparison of only 482 minimum number of SNPs, it is cost-effective and can reduce the time required for kinship identification, and accordingly, the 482 SNP markers according to the present invention are useful in the field of forensic science, legal medicine, or criminal investigation.

Consequently, the two SNP panels investigated in the present invention for kinship estimation seem to be useful in distinguishing first-d-r individuals from the unrelated individuals. The 918-SNP panel is expected to be able to distinguish first-d-r and second-d-r from each other. However, it is difficult to distinguish between first-d-r and second-d-r by using the 482-SNP panel, and it would be difficult to apply either of the two panels to distinguish third-d-r from the unrelated individuals. It is considered that this limitation could be overcome by increasing the number of SNP markers, such as by simultaneously using both the 918-SNP panel and the 482-SNP panel, or simultaneously using a part of the 482-SNP panel to the entirety of the 918-SNP panel, or simultaneously using a part of the 918-SNP panel to the entirety of the 482-SNP panel.

Those skilled in the art would appreciate that a part or all of the SNP markers disclosed in the present invention for kinship identification in Korean can be used to clearly distinguish individuals in first-d-r from those who are not first-d-r.

Accordingly, the present invention provides at least two SNP panels configured by identifying the genetic characteristics of Koreans, and can measure and estimate the genetic distance within a population by comparing the 918-SNP panel and/or the 482-SNP panel, and thus is expected to be useful in forensic investigations.

In addition, since the SNP panels according to the present invention can process raw data of a large number of individuals simultaneously, in cases where all of immediate family members have died a long ago or in massive disasters and therefore no current technology can analyze kinship, or even when the genetic distance among the surviving family members is distant, the SNP panels according to the present invention can be used to estimate kinship and thus is expected to resolve past affairs.

### REFERENCES

Anderson, C. A., Pettersson, F. H., Clarke, G. M., Cardon, L. R., Morris, A. P., & Zondervan, K. T. (2010). Data quality control in genetic case-control association studies. Nature protocols, 5(9), 1564-1573.
Andrews, S. (2010). FastQC: a quality control tool for high throughput sequence data. Cambridge, UK: Babraham Institute.
Fan H, Chu J Y. (2007). A brief review of short tandem repeat mutation. Genomics Proteomics Bioinformatics. February; 5(1):7-14.
Li, H. (2013). Aligning sequence reads, clone sequences and assembly contigs with BWA-MEM. arXiv preprint arXiv:1303.3997.
Ward, L. D., & Kellis, M. (2016). HaploReg v 4: systematic mining of putative causal variants, cell types, regulators and target genes for human complex traits and disease. Nucleic acids research, 44(D1), D877-D881.

Pedersen, B. S., Bhetariya, P. J., Brown, J., Kravitz, S. N., Marth, G., Jensen, R. L., . . . & Quinlan, A. R. (2020). Somalier: rapid relatedness estimation for cancer and germline studies using efficient genome sketches. Genome medicine, 12(1), 1-9.

1. A composition for kinship identification in Korean, the composition comprising:
1) an agent for amplifying or detecting a single nucleotide polymorphism (SNP) located at position 101 in at least one sequence selected from the group consisting of nucleotide sequences set forth as SEQ ID NO: 1 to SEQ ID NO: 918;
2) an agent for amplifying or detecting a single nucleotide polymorphism (SNP) located at position 101 in at least one sequence selected from the group consisting of nucleotide sequences set forth as SEQ ID NO: 919 to SEQ ID NO: 1400; or
3) an agent for amplifying or detecting a single nucleotide polymorphism (SNP) located at position 101 in at least one sequence selected from the group consisting of nucleotide sequences set forth as SEQ ID NO: 1 to SEQ ID NO: 1400.

2. The composition for kinship identification in Korean of claim 1, wherein the agent is a primer, a probe, or a mixture thereof.

3. The composition for kinship identification in Korean of claim 1, wherein the kinship is any one of relationships selected from the group consisting of parent, child, brother, sister, and sibling, with respect to a subject.

4. A method of identifying a kinship in Korean, the method comprising:
(1) identifying a nucleotide of an SNP located at position 101 in at least one sequence selected from the group consisting of nucleotide sequences set forth as SEQ ID NO: 1 to SEQ ID NO: 918, in samples isolated from two or more individuals whose kinship is to be identified; or
(2) identifying a nucleotide of an SNP located at position 101 in at least one sequence selected from the group consisting of nucleotide sequences set forth as SEQ ID NO: 919 to SEQ ID NO: 1400, in samples isolated from two or more individuals whose kinship is to be identified.

5. The method of claim 4, further comprising:
in case said (1), identifying the nucleotide of the SNP located at position 101 in at least one sequence selected from the group consisting of nucleotide sequences set forth as SEQ ID NO: 919 to SEQ ID NO: 1400; or
in case said (2), identifying the nucleotide of the SNP located at position 101 in at least one sequence selected from the group consisting of nucleotide sequences set forth as SEQ ID NO: 1 to SEQ ID NO: 918.

6. The method of claim 4, wherein the kinship is any one of relationships selected from the group consisting of parent, child, brother, sister, and sibling, with respect to a subject.

7. The method of claim 4, wherein the identifying the nucleotide at an SNP is amplifying or detecting the SNP by using a primer, a probe, or a mixture thereof.

8. The method of claim 7, further comprising, after the identifying the nucleotide at an SNP, making pairwise comparison of each SNP nucleotide in each sample.

9. The method of claim 8, wherein the making pairwise comparison of each SNP nucleotide in each sample comprises:
(a) i) when all nucleotides of the SNP identified from two alleles in two samples being pairwise compared are identical, assigning an IBS (identity by state) score of 2 to the SNP, ii) when only one nucleotide of the SNP is identical between two alleles in two samples being pairwise compared, assigning an IBS score of 1 to the SNP, or iii) when all nucleotides of the SNP identified from two alleles in two samples being pairwise compared are different, assigning an IBS score of 0 to the SNP; and
(b) obtaining an average of IBS scores of all the SNPs compared pairwise.

10. The method of claim 9, when the average of IBS score from the (b) obtaining an average is 0.300 to 0.700, there is provided information indicating that the two individuals from which the two samples pairwise compared were isolated are in any one of kinship selected from the group consisting of parent, child, brother, sister, and sibling; or
when the average of IBS score from the (b) obtaining an average is less than 0.300, there is provided information indicating that the two individuals from which the two samples pairwise compared were isolated are not in any one of kinship selected from the group consisting of parent, child, brother, sister, and sibling.

11. A method of developing an SNP marker for kinship identification, the method comprising extracting, from the human genome database, an SNP characterized by at least one of the following features:
an SNP having a p value of 0.05 or more at Hardy-Weinberg equilibrium (HWE);
an SNP that is not present within a genomic region or within 100 kbp upstream or downstream of the genomic region;
an SNP having a variant allele frequency of 0.3 to 0.7;
an SNP not present in linkage disequilibrium (LD); and
an SNP not present in repeated regions.

12. The method of claim 11, wherein the genomic region is an exon or a coding sequence.

13. The method of claim 11, wherein the method extracts an SNP having a variant allele frequency of 0.4 to 0.6.
